(19) United States
(12) Patent Application Publication
(10) Pub. No.: US 2025/0188037 A1
(43) Pub. Date: Jun. 12, 2025

(54) N-(PYRROLIDIN-3-YL OR PIPERIDIN-4-YL)ACETAMIDE DERIVATIVES

(71) Applicant: Takeda Pharmaceutical Company Limited, Osaka (JP)

(72) Inventors: Maria HOPKINS, San Diego, CA (US); Andre KIRYANOV, San Diego, CA (US); Kristin SCHLEICHER, San Diego, CA (US); Mingnam TANG, San Diego, CA (US); Feng ZHOU, San Diego, CA (US); Zacharia CHERUVALLATH, San Diego, CA (US); Scott OLSEN, San Diego, CA (US)

(21) Appl. No.: 18/852,426

(22) PCT Filed: Mar. 29, 2023
(86) PCT No.: PCT/IB2023/053132

§ 371 Date: Sep. 27, 2024

Related U.S. Application Data
(60) Provisional application No. 63/325,452, filed on Mar. 30, 2022.

(51) Int. Cl. C07D 221/20; A61K 31/403; A61K 31/407; A61K 31/438; A61K 31/4468; A61K 31/4545; A61K 45/06; C07D 207/14; C07D 209/54; C07D 211/58; C07D 401/12; C07D 487/04

(52) U.S. Cl. CPC C07D 221/20; A61K 31/403; A61K 31/407; A61K 31/438; A61K 31/4468; A61K 31/4545; A61K 45/06; C07D 207/14; C07D 209/54; C07D 211/58; C07D 401/12; C07D 487/04

(57) ABSTRACT

Disclosed are compounds of Formula 1, and pharmaceutically acceptable salts thereof, wherein L⁶, n, R², R³, R⁴, R⁵, R⁷, R⁸, R⁹, R¹⁰, R¹¹, R¹², R¹³, R¹⁴, R¹⁵ and X¹ are defined in the specification. This disclosure also relates to materials and methods for preparing compounds of Formula 1, to pharmaceutical compositions which contain them, and to their use for treating diseases, disorders, and conditions associated with SSTR4.

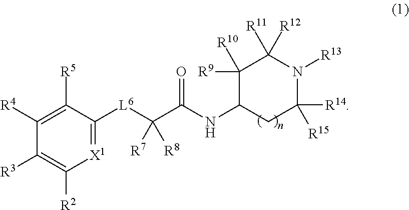

### FIELD OF THE INVENTION

This invention relates to N-(pyrrolidine-3-yl or piperidin-4-yl)acetamide derivatives which are modulators of somatostatin receptor 4 (SSTR4), to pharmaceutical compositions which contain them, and to their use to treat diseases, disorders, and conditions associated with SSTR4, including Alzheimer's disease.

### BACKGROUND OF THE INVENTION

Somatostatin receptor 4 (SSTR4) is a G-protein coupled receptor for the peptide somatostatin. SSTR4 is coupled with Gi, inhibitory G protein, which inhibits production of cyclic AMP. SSTR4 is abundantly expressed in the central nervous system (CNS) and to a lesser extent in the dorsal root ganglia and intestine. See M. A. Meyer, “Highly Expressed Genes within Hippocampal Sector CA1: Implications for the Physiology of Memory,” Neurology International 6(2):5388 (2014). SSTR4 is highly conserved among different species. For example, human, mouse, and rat SSTR4 protein sequences share greater than 87% identity at the amino acid level. These factors-predominant expression in the brain and high degree of sequence homology across different species-suggest that SSTR4 has an important role in physiology.

Experiments using bacTRAP technology indicate SSTR4 has its strongest expression in the pyramidal neurons in the cortex and in the CA1 region of the hippocampus. This CNS expression is conserved in humans, non-human primates, and mice. The hippocampus is important for learning and memory. See L. R. Squire and A. J. Dede, “Conscious and Unconscious Memory Systems,” Cold Spring Harbor Perspectives in Biology 7:a021667 (2015). Indeed, the CA1 region of the hippocampus is the last station in the trisynaptic circuit that governs learning. This circuit starts in the entorhinal cortex, which also contains SSTR4, extends into the dentate gyrus, then into CA3, and finally reaches the CA1 region of the hippocampus. CA1 projects out of the hippocampus through the subiculum. This circuit encodes all types of information from the external world in order to generate memories and to learn new knowledge.

Alzheimer's disease is characterized by degeneration of neurons within this circuitry, mainly in the entorhinal cortex and CA1 region of the hippocampus. See A. Serrano-Pozo et al., “Neuropathological Alterations in Alzheimer Disease,” Cold Spring Harbor Perspectives in Medicine 1:a006189 (2011). In addition, hippocampal sst4 appears to selectively control the use of cognitive strategies by switching from hippocampus-based multiple associations to simple striatum-based behavioral responses. See F. Gastambide et al., “Hippocampal SSTR4 Somatostatin Receptors Control the Selection of Memory Strategies,” Psychopharmacology (Berl) 202(1-3):153-63 (2009). This finding provides a strong basis for using SSTR4 agonists as a pharmacological approach to improve striatum-based learning. Id.

Moreover, recent studies also point to hyperactivity of the hippocampus as a main driver for disease progression as well as impairment of cognitive abilities in Alzheimer's patients. See M. A. Busche et al., “Decreased Amyloid-β and Increased Neuronal Hyperactivity by Immunotherapy in Alzheimer's Models,” Nature Neuroscience 18(12):1725-27 (2015); see also K. Yamamoto et al., “Chronic Optogenetic Activation Augments Aβ Pathology in a Mouse Model of Alzheimer Disease,” Cell Reports 11(6):859-65 (2015). Activation of SSTR4 receptor has been shown to play a role in controlling neuronal activity. See C. Qiu et al., “Somatostatin Receptor Subtype 4 Couples to the M-Current to Regulate Seizures,” Journal of Neuroscience 28(14):3567-76 (2008). Thus, agonists for the receptor will likely represent good pharmacological tools to inhibit and control neuronal activity in the cortex and hippocampus.

SSTR4 agonists are expected to be useful for treating Alzheimer's disease and other CNS disorders such as epilepsy and depression.

### SUMMARY OF THE INVENTION

This invention provides N-(pyrrolidine-3-yl or piperidin-4-yl)acetamide derivatives and pharmaceutically acceptable salts thereof. This invention also provides pharmaceutical compositions that contain the N-(pyrrolidine-3-yl or piperidin-4-yl)acetamide derivatives and provides for their use to treat diseases, disorders and conditions associated with SSTR4, including Alzheimer's disease and other CNS disorders.

One aspect of the invention provides a compound of Formula 1:

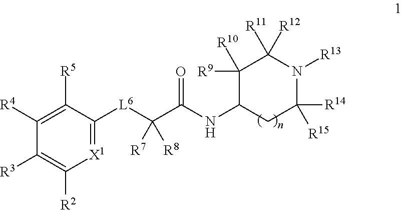

or a pharmaceutically acceptable salt thereof in which:
X¹ is selected from N and CR¹;
n is selected from 0 and 1;
R¹, R², R³, R⁴ and Rare each independently selected from
   (i) hydrogen, halo, hydroxy and cyano; and
   (ii) C₁₋₃ alkyl, C₁₋₃ alkoxy and C₃₋₆ cycloalkyl, each unsubstituted or substituted with 1 to 3 substituents independently selected from halo;
L⁶ is selected from —CH₂—, —N(R⁶)—, *—N(R⁶)CH₂— and —O—, wherein R⁶ is selected from hydrogen and C₁₋₃ alkyl, and * represents the point of attachment to an aromatic ring carbon atom;
R⁷ and R⁸ are each independently selected from halo and C₁₋₃ alkyl, provided R⁷ and R⁸ are not both methyl; or R⁷ and R⁸, together with the carbon atom to which they are attached, form a cyclopropylidene;
R⁹ and R¹⁰ are each independently selected from
   (i) hydrogen and halo; and
   (ii) C₁₋₃ alkyl and phenyl, each unsubstituted or substituted with 1 to 3 substituents independently selected from halo; or
R⁹ and R¹⁰, together with the carbon atom to which they are attached, form a cyclopropylidene;
R¹¹ and R¹² are each independently selected from hydrogen, halo and C₁₋₃ alkyl which is unsubstituted or substituted with 1 to 3 substituents independently selected from halo, provided no more than one of R¹¹ and R¹² is methyl; and
(a) R¹³ is selected from hydrogen and C₁₋₃ alkyl; and
   R¹⁴ and R¹⁵ are each independently selected from hydrogen, halo, and C₁₋₃ alkyl which is unsubstituted or substituted with 1 to 3 substituents independently selected from halo, provided no more than one of R¹⁴ and R¹⁵ is methyl; or
(b) R¹³ and R¹⁴ together form a propane-1,3-diyl bridging the nitrogen and carbon atoms to which they are respectively attached; and
   R¹⁵ is selected from hydrogen, halo, and C₁₋₃ alkyl which is unsubstituted or substituted with 1 to 3 substituents independently selected from halo;
      provided
(i) if X¹ is CR¹, L⁶ is —CH₂—, and R⁷ and R⁸ form a cyclopropylidene, then at least one of R¹, R², R³, R⁴, R⁵, R⁹, R¹⁰, R¹¹, R¹², R¹³, R¹⁴ and R¹⁵ is not hydrogen;
(ii) if X¹ is CR¹, L⁶ is —CH₂—, n is 0, R¹, R², R⁴, R⁵, R¹¹, R¹², R¹⁴ and R¹⁵ are each hydrogen, R⁷ and R⁸ form a cyclopropylidene, and R⁹, R¹⁰ and R¹³ are each methyl, then R³ is not fluoro or hydroxy;
(iii) if X¹ is CR¹, L⁶ is —CH₂—, n is 0, R¹ is fluoro, R², R³, R⁴, R⁵, R¹¹, R¹², R¹⁴ and R¹⁵ are each hydrogen, and R⁷ and R⁸ form a cyclopropylidene, then R⁹, R¹⁰ and R¹³ are not all hydrogen and not all methyl;
(iv) if X¹ is CR¹, L⁶ is —CH₂—, n is 1, R¹, R², R³, R⁴, R⁵, R¹⁰, R¹¹, R¹², R¹³, R¹⁴ and R¹⁵ are each hydrogen, and R⁷ and R⁸ form a cyclopropylidene, then R⁹ is not methyl;
(v) if X¹ is CR¹, L⁶ is —CH₂—, n is 1, R¹, R², R³, R⁴, R⁵, R⁹, R¹⁰, R¹², R¹³, R¹⁴ and R¹⁵ are each hydrogen, and R⁷ and R⁸ form a cyclopropylidene, then R¹¹ is not methyl;
(vi) if X¹ is CR¹, n is 0, R¹, R², R³, R⁴, R⁵, R¹¹, R¹², R¹⁴ and R¹⁵ are each hydrogen, R⁷ and R⁸ form a cyclopropylidene, R⁹ and R¹⁰ are each methyl, and R¹³ is ethyl, then L⁶ is not —O— or —NH—;
(vii) if X¹ is CR¹, L⁶ is —CH₂—, n is 0, R¹, R², R⁴, R⁵, R¹¹, R¹², R¹⁴ and R¹⁵ are each hydrogen, R⁷ and R⁸ form a cyclopropylidene, R⁹ and R¹⁰ are both methyl, and R¹³ is ethyl, then R³ is not hydroxy;
(viii) if X¹ is CR¹, L⁶ is —CH₂—, n is 1, R⁷ and R⁸ form a cyclopropylidene, and R¹³ and R¹⁴ together form a propane-1,3-diyl bridging the nitrogen and carbon atoms to which they are respectively attached, then at least one of R¹, R², R³, R⁴, R⁵, R⁹, R¹⁰, R¹¹, R¹² and R¹⁵ is not hydrogen;
(ix) if X¹ is CR¹, L⁶ is —CH₂—, n is 1, R⁷ and R⁸ form a cyclopropylidene, and R¹³ and R¹⁴ together form a propane-1,3-diyl bridging the nitrogen and carbon atoms to which they are respectively attached, and R², R³, R⁴, R⁵, R⁹, R¹⁰, R¹¹, R¹² and R¹⁵ are each hydrogen, then R¹ is not fluoro; and
(x) if X¹ is CR¹, L⁶ is —CH₂—, n is 1, R⁷ and R⁸ form a cyclopropylidene, and R¹³ and R¹⁴ together form a propane-1,3-diyl bridging the nitrogen and carbon atoms to which they are respectively attached, and R¹, R², R⁴, R⁵, R⁹, R¹⁰, R¹¹, R¹² and R¹⁵ are each hydrogen, then R³ is not fluoro.

Another aspect of the invention provides a compound which is selected from the group of compounds described in the examples and their pharmaceutically acceptable salts.

A further aspect of the invention provides a compound or pharmaceutically acceptable salt as defined in the preceding paragraphs for use as a medicament.

An additional aspect of the invention provides a compound of Formula 1 or a pharmaceutically acceptable salt thereof for use as a medicament in which:
X¹ is selected from N and CR¹;
n is selected from 0 and 1;
R¹, R², R³, R⁴ and R⁵ are each independently selected from
   (i) hydrogen, halo, hydroxy and cyano; and
   (ii) C₁₋₃ alkyl, C₁₋₃ alkoxy and C₃₋₆ cycloalkyl, each unsubstituted or substituted with 1 to 3 substituents independently selected from halo;
L⁶ is selected from —CH₂—, —N(R⁶)—, *—N(R⁶)CH₂— and —O—, wherein R⁶ is selected from hydrogen and C₁₋₃ alkyl, and * represents the point of attachment to an aromatic ring carbon atom;
R⁷ and R⁸ are each independently selected from hydrogen, halo and C₁₋₃ alkyl, wherein at least one of R⁷ and R⁸ is not hydrogen; or R⁷ and R⁸, together with the carbon atom to which they are attached, form a C₃₋₆ cycloalkylidene;
R⁹ and R¹⁰ are each independently selected from
   (i) hydrogen and halo; and
   (ii) C₁₋₃ alkyl and phenyl, each unsubstituted or substituted with 1 to 3 substituents independently selected from halo; or
R⁹ and R¹⁰, together with the carbon atom to which they are attached, form a cyclopropylidene;
R¹¹ and R¹² are each independently selected from hydrogen, halo and C₁₋₃ alkyl which is unsubstituted or substituted with 1 to 3 substituents independently selected from halo, provided no more than one of R¹¹ and R¹² is methyl; and
(a) R¹³ is selected from hydrogen and C₁₋₃ alkyl; and
   R¹⁴ and R¹⁵ are each independently selected from hydrogen, halo, and C₁₋₃ alkyl which is unsubstituted or substituted with 1 to 3 substituents independently selected from halo, provided no more than one of R¹⁴ and R¹⁵ is methyl; or
(b) R¹³ and R¹⁴ together form a propane-1,3-diyl bridging the nitrogen and carbon atoms to which they are respectively attached; and
   R¹⁵ is selected from hydrogen, halo, and C₁₋₃ alkyl which is unsubstituted or substituted with 1 to 3 substituents independently selected from halo;
      provided
(i) if X is CR¹, n is 1, L⁶ is —O—, R⁷ is methyl and R⁸ is hydrogen, then at least one of R¹, R², R³, R⁴, R⁵, R⁹, R¹⁰, R¹¹, R¹², R¹³, R¹⁴ or R¹⁵ is not hydrogen;
(ii) if X is CR¹, n is 1, L⁶ is —O—, R⁷ and R⁸ are both methyl, and R¹, R², R⁴, R⁵, R⁹, R¹⁰, R¹¹, R¹², R¹³, R¹⁴ and R¹⁵ are each hydrogen, then R³ is not chloro; and
(iii) if X is CR¹, n is 1, L⁶ is —O—, R⁷ is methyl or ethyl, R¹, R², R³, R⁴, R⁵, R⁸, R¹⁰, R¹¹, R¹², R¹³, R¹⁴ and R¹⁵ are each hydrogen, then R⁹ is not unsubstituted phenyl.

Another aspect of the invention provides a pharmaceutical composition which includes a compound of Formula 1 or a pharmaceutically acceptable salt thereof, or any one of the compounds or pharmaceutically acceptable salts defined in the preceding paragraphs; and a pharmaceutically acceptable excipient.

A further aspect of the invention provides a compound of Formula 1 or a pharmaceutically acceptable salt thereof, or any one of the compounds or pharmaceutically acceptable salts defined in the preceding paragraphs, for treatment of a disease, disorder or condition associated with SSTR4.

An additional aspect of the invention provides a use of a compound of Formula 1 or a pharmaceutically acceptable salt thereof, or any one of the compounds or pharmaceutically acceptable salts defined in the preceding paragraphs, for the manufacture of a medicament for the treatment of a disease, disorder or condition associated with SSTR4.

Another aspect of the invention provides a method of treating a disease, disorder or condition associated with SSTR4, the method comprising administering to the subject an effective amount of a compound of Formula 1 or a pharmaceutically acceptable salt thereof, or any one of the compounds or pharmaceutically acceptable salts defined in the preceding paragraphs.

A further aspect of the invention provides a method of treating a disease, disorder or condition in a subject, the method comprising administering to the subject an effective amount of a compound of Formula 1 or a pharmaceutically acceptable salt thereof, or any one of the compounds or pharmaceutically acceptable salts defined in the preceding paragraphs, wherein the disease, disorder or condition is selected from Alzheimer's disease, depression, anxiety, schizophrenia, bipolar disorder, autism, epilepsy, pain, and hyperactivity disorder.

An additional aspect of the invention provides an effective amount of a compound of Formula 1 or a pharmaceutically acceptable salt thereof, or any one of the compounds or pharmaceutically acceptable salts defined in the preceding paragraphs; and at least one additional pharmacologically active agent.

### DETAILED DESCRIPTION OF THE INVENTION

Unless otherwise indicated, this disclosure uses definitions provided below.

“Substituted,” when used in connection with a chemical substituent or moiety (e.g., a C₁₋₆ alkyl group), means that one or more hydrogen atoms of the substituent or moiety have been replaced with one or more non-hydrogen atoms or groups, provided valence requirements are met and a chemically stable compound results from the substitution.

“About” or “approximately,” when used in connection with a measurable numerical variable, refers to the indicated value of the variable and to all values of the variable that are within the experimental error of the indicated value or within ±10 percent of the indicated value, whichever is greater.

“Alkyl” refers to straight chain and branched saturated hydrocarbon groups, generally having a specified number of carbon atoms (e.g., C₁₋₄ alkyl refers to an alkyl group having 1 to 4 (i.e., 1, 2, 3 or 4) carbon atoms, C₁₋₆ alkyl refers to an alkyl group having 1 to 6 carbon atoms, and so on). Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkanediyl” refers to divalent alkyl groups, where alkyl is defined above, and generally having a specified number of carbon atoms (e.g., C₁₋₄ alkanediyl refers to an alkanediyl group having 1 to 4 (i.e., 1, 2, 3 or 4) carbon atoms, C₁₋₆ alkanediyl refers to an alkanediyl group having 1 to 6 carbon atoms, and so on). Examples of alkanediyl groups include methylene, ethane-1,1-diyl, ethane-1,2-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, isobutane-1,3-diyl, isobutane-1,1-diyl, isobutane-1,2-diyl, and the like.

“Alkenyl” refers to straight chain and branched hydrocarbon groups having one or more carbon-carbon double bonds, and generally having a specified number of carbon atoms. Examples of alkenyl groups include ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include ethynyl, 1-propyn-1-yl, 2-propyn-1-yl, 1-butyn-1-yl, 3-butyn-1-yl, 3-butyn-2-yl, 2-butyn-1-yl, and the like.

“Alkoxy” refers to straight chain and branched saturated hydrocarbon groups attached through an oxygen atom, generally having a specified number of carbon atoms (e.g., C₁₋₄ alkoxy refers to an alkoxy group having 1 to 4 (i.e., 1, 2, 3 or 4) carbon atoms, C₁₋₆ alkoxy refers to an alkoxy group having 1 to 6 carbon atoms, and so on). Examples of alkoxy groups include methoxy, ethoxy, n-propoxy, i-propoxy, n-butoxy, s-butoxy, i-butoxy, t-butoxy, pent-1-yloxy, pent-2-yloxy, pent-3-yloxy, 3-methylbut-1-yloxy, 3-methylbut-2-yloxy, 2-methylbut-2-yloxy, 2,2,2-trimethyleth-1-yloxy, n-hexoxy, and the like.

“Halo,” “halogen” and “halogeno” may be used interchangeably and refer to fluoro, chloro, bromo, and iodo.

“Haloalkyl,” “haloalkenyl,” and “haloalkynyl,” refer, respectively, to alkyl, alkenyl, and alkynyl groups substituted with one or more halogen atoms, where alkyl, alkenyl, and alkynyl are defined above, and generally having a specified number of carbon atoms. Examples of haloalkyl groups include fluoromethyl, difluoromethyl, trifluoromethyl, chloromethyl, dichloromethyl, trichloromethyl, 1-fluoroethyl, 1,1-difluoroethyl, 1-chloroethyl, 1,1-dichloroethyl, 1-fluoro-1-methylethyl, 1-chloro-1-methylethyl, and the like.

“Cycloalkyl” refers to saturated monocyclic and bicyclic hydrocarbon groups, generally having a specified number of carbon atoms that comprise the ring or rings (e.g., C₃₋₈ cycloalkyl refers to a cycloalkyl group having 3 to 8 carbon atoms as ring members). Bicyclic hydrocarbon groups may include isolated rings (two rings sharing no carbon atoms), spiro rings (two rings sharing one carbon atom), fused rings (two rings sharing two carbon atoms and the bond between the two common carbon atoms), and bridged rings (two rings sharing two carbon atoms, but not a common bond). The cycloalkyl group may be attached through any ring atom unless such attachment would violate valence requirements, and where indicated, may optionally include one or more non-hydrogen substituents unless such substitution would violate valence requirements.

Examples of monocyclic cycloalkyl groups include cyclopropyl, cyclobutyl, cyclopentyl, cyclohexyl, and the like. Examples of fused bicyclic cycloalkyl groups include bicyclo[2.1.0]pentanyl (i.e., bicyclo[2.1.0]pentan-1-yl, bicyclo[2.1.0]pentan-2-yl, and bicyclo[2.1.0]pentan-5-yl), bicyclo[3.1.0]hexanyl, bicyclo[3.2.0]heptanyl, bicyclo[4.1.0]heptanyl, bicyclo[3.3.0]octanyl, bicyclo[4.2.0]octanyl, bicyclo[4.3.0]nonanyl, bicyclo[4.4.0]decanyl, and the like. Examples of bridged cycloalkyl groups include bicyclo[2.1.1]hexanyl, bicyclo[2.2.1]heptanyl, bicyclo[3.1.1]heptanyl, bicyclo[2.2.2]octanyl, bicyclo[3.2.1]octanyl, bicyclo[4.1.1]octanyl, bicyclo[3.3.1]nonanyl, bicyclo[4.2.1]nonanyl, bicyclo[3.3.2]decanyl, bicyclo[4.2.2]decanyl, bicyclo[4.3.1]decanyl, bicyclo[3.3.3]undecanyl, bicyclo[4.3.2]undecanyl, bicyclo[4.3.3]dodecanyl, and the like. Examples of spiro cycloalkyl groups include spiro[3.3]heptanyl, spiro[2.4]heptanyl, spiro[3.4]octanyl, spiro[2.5]octanyl, spiro[3.5]nonanyl, and the like. Examples of isolated bicyclic cycloalkyl groups include those derived from bi(cyclobutane), cyclobutanecyclopentane, bi(cyclopentane), cyclobutanecyclohexane, cyclopentanecyclohexane, bi(cyclohexane), etc.

“Cycloalkanediyl” refers to divalent cycloalkyl groups, where cycloalkyl is defined above, and generally having a specified number of carbon atoms (e.g., C₃₋₅ cycloalkanediyl refers to a cycloalkanediyl group having 3 to 5 (i.e., 3, 4 or 5) carbon atoms, C₃₋₆ cycloalkanediyl refers to a cycloalkanediyl group having 3 to 6 carbon atoms, and so on). Examples of cycloalkanediyl groups include cyclopropane-1,1-diyl, cyclopropane-1,2-diyl, cyclobutane-1,1-diyl, cyclobutane-1,2-diyl, and the like.

“Cycloalkylidene” refers to divalent monocyclic cycloalkyl groups, where cycloalkyl is defined above, which are attached through a single carbon atom of the group, and generally having a specified number of carbon atoms that comprise the ring (e.g., C₃₋₆ cycloalkylidene refers to a cycloalkylidene group having 3 to 6 carbon atoms as ring members). Examples include cyclopropylidene, cyclobutylidene, cyclopentylidene, and cyclohexylidene.

“Cycloalkenyl” refers to partially unsaturated monocyclic and bicyclic hydrocarbon groups, generally having a specified number of carbon atoms that comprise the ring or rings. As with cycloalkyl groups, the bicyclic cycloalkenyl groups may include isolated, spiro, fused, or bridged rings. Similarly, the cycloalkenyl group may be attached through any ring atom, and where indicated, may optionally include one or more non-hydrogen substituents unless such attachment or substitution would violate valence requirements. Examples of cycloalkenyl groups include the partially unsaturated analogs of the cycloalkyl groups described above, such as cyclobutenyl (i.e., cyclobuten-1-yl and cyclobuten-3-yl), cyclopentenyl, cyclohexenyl, bicyclo[2.2.1]hept-2-enyl, and the like.

“Aryl” refers to fully unsaturated monocyclic aromatic hydrocarbons and to polycyclic hydrocarbons having at least one aromatic ring, both monocyclic and polycyclic aryl groups generally having a specified number of carbon atoms that comprise their ring members (e.g., C₆₋₁₄ aryl refers to an aryl group having 6 to 14 carbon atoms as ring members). The group may be attached through any ring atom, and where indicated, may optionally include one or more non-hydrogen substituents unless such attachment or substitution would violate valence requirements. Examples of aryl groups include phenyl, biphenyl, cyclobutabenzenyl, indenyl, naphthalenyl, benzocycloheptanyl, biphenylenyl, fluorenyl, groups derived from cycloheptatriene cation, and the like.

“Arylene” refers to divalent aryl groups, where aryl is defined above. Examples of arylene groups include o-phenylene (i.e., benzene-1,2-diyl).

“Heterocycle” and “heterocyclyl” may be used interchangeably and refer to saturated or partially unsaturated monocyclic or bicyclic groups having ring atoms composed of carbon atoms and 1 to 4 heteroatoms independently selected from nitrogen, oxygen, and sulfur. Both the monocyclic and bicyclic groups generally have a specified number of carbon atoms in their ring or rings (e.g., C₂₋₆ heterocyclyl refers to a heterocyclyl group having 2 to 6 carbon atoms and 1 to 4 heteroatoms as ring members). As with bicyclic cycloalkyl groups, bicyclic heterocyclyl groups may include isolated rings, spiro rings, fused rings, and bridged rings. The heterocyclyl group may be attached through any ring atom, and where indicated, may optionally include one or more non-hydrogen substituents unless such attachment or substitution would violate valence requirements or result in a chemically unstable compound. Examples of heterocyclyl groups include oxiranyl, thiiranyl, aziridinyl (e.g., aziridin-1-yl and aziridin-2-yl), oxetanyl, thietanyl, azetidinyl, tetrahydrofuranyl, tetrahydrothienyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thiazepanyl, 1,4-diazepanyl, 3,4-dihydro-2H-pyranyl, 3,6-dihydro-2H-pyranyl, 2H-pyranyl, 1,2-dihydropyridinyl, 1,2,3,4-tetrahydropyridinyl, 1,2,5,6-tetrahydropyridinyl, 1,6-dihydropyrimidinyl, 1,2,3,4-tetrahydropyrimidinyl, and 1,2-dihydropyrazolo[1,5-d][1,2,4]triazinyl.

“Heterocycle-diyl” refers to heterocyclyl groups which are attached through two ring atoms of the group, where heterocyclyl is defined above. They generally have a specified number of carbon atoms in their ring or rings (e.g., C₂₋₆ heterocycle-diyl refers to a heterocycle-diyl group having 2 to 6 carbon atoms and 1 to 4 heteroatoms as ring members). Examples of heterocycle-diyl groups include the multivalent analogs of the heterocycle groups described above, such as morpholine-3,4-diyl, pyrrolidine-1,2-diyl, 1-pyrrolidinyl-2-ylidene, 1-pyridinyl-2-ylidene, 1-(4H)-pyrazolyl-5-ylidene, 1-(3H)-imidazolyl-2-ylidene, 3-oxazolyl-2-ylidene, 1-piperidinyl-2-ylidene, 1-piperazinyl-6-ylidene, and the like.

“Heteroaromatic” and “heteroaryl” may be used interchangeably and refer to unsaturated monocyclic aromatic groups and to polycyclic groups having at least one aromatic ring, each of the groups having ring atoms composed of carbon atoms and 1 to 4 heteroatoms independently selected from nitrogen, oxygen, and sulfur. Both the monocyclic and polycyclic groups generally have a specified number of carbon atoms as ring members (e.g., C₁₋₉ heteroaryl refers to a heteroaryl group having 1 to 9 carbon atoms and 1 to 4 heteroatoms as ring members) and may include any bicyclic group in which any of the above-listed monocyclic heterocycles are fused to a benzene ring. The heteroaryl group may be attached through any ring atom (or ring atoms for fused rings), and where indicated, may optionally include one or more non-hydrogen substituents unless such attachment or substitution would violate valence requirements or result in a chemically unstable compound. Examples of heteroaryl groups include monocyclic groups such as pyrrolyl (e.g., pyrrol-1-yl, pyrrol-2-yl, and pyrrol-3-yl), furanyl, thienyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, and pyrazinyl.

Examples of heteroaryl groups also include bicyclic groups such as benzofuranyl, isobenzofuranyl, benzothienyl, benzo[c]thienyl, 1H-indolyl, 3H-indolyl, isoindolyl, 1H-isoindolyl, indolinyl, isoindolinyl, benzimidazolyl, 1H-indazolyl, 2H-indazolyl, benzotriazolyl, 1H-pyrrolo[2,3-b]pyridinyl, 1H-pyrrolo[2,3-c]pyridinyl, 1H-pyrrolo[3,2-c]pyridinyl, 1H-pyrrolo[3,2-b]pyridinyl, 3H-imidazo[4,5-b]pyridinyl, 3H-imidazo[4,5-c]pyridinyl, 1H-pyrazolo[4,3-b]pyridinyl, 1H-pyrazolo[4,3-c]pyridinyl, 1H-pyrazolo[3,4-c]pyridinyl, 1H-pyrazolo[3,4-b]pyridinyl, 7H-purinyl, indolizinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-d]pyrimidinyl, 1,2,3,4-tetrahydropyrido[2,3-b]pyrazinyl, 2,3-dihydrobenzo[b][1,4]dioxinyl, 3,4-dihydro-2H-pyrido[3,2-b][1,4]oxazinyl, 2,3-dihydro-1H-benzo[d]imidazolyl, benzo[d]thiazolyl, 2,3-dihydro-1H-pyrrolo[2,3-b]pyridinyl, [1,2,4]triazolo[1,5-a]pyridinyl, 2,3-dihydro-1H-imidazo[4,5-b]pyridinyl, tetrazolo[1,5-a]pyridinyl, 7H-pyrrolo[2,3-d]pyrimidinyl, pyrazolo[1,5-a]pyrimidinyl, imidazo[1,2-a]pyrimidinyl, 4,5-dihydro-1H-pyrazolo[3,4-d]pyrimidinyl, 2,3,6,7-tetrahydro-1H-purinyl, 5H-pyrrolo[2,3-b]pyrazinyl, imidazo[1,2-a]pyrazinyl, imidazo[1,2-b]pyridazinyl, and 4,5,6,7-tetrahydropyrazolo[1,5-a]pyrazinyl.

“Heteroarylene” refers to heteroaryl groups which are attached through two ring atoms of the group, where heteroaryl is defined above. They generally have a specified number of carbon atoms in their ring or rings (e.g., C₃₋₅ heteroarylene refers to a heteroarylene group having 3 to 5 carbon atoms and 1 to 4 heteroatoms as ring members). Examples of heteroarylene groups include the multivalent analogs of the heteroaryl groups described above, such as pyridine-2,3-diyl, pyridine-3,4-diyl, pyrazole-4,5-diyl, pyrazole-3,4-diyl, and the like.

“Oxo” refers to a double bonded oxygen (═O).

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts); sulfonates, including alkylsulfonates (e.g., mesylate), fluoroalkylsulfonates (e.g., triflate, hexaflate, nonaflate, and tresylate), and arylsulfonates (e.g., tosylate, brosylate, closylate, and nosylate). Others include carbonates, halide ions, carboxylate anions, phenolate ions, and alkoxides. Some stronger bases, such as NH₂⁻ and OH⁻ can be made better leaving groups by treatment with an acid. Common electrofugal leaving groups include the proton, CO₂, and metals.

“Opposite enantiomer” refers to a molecule that is a non-superimposable mirror image of a reference molecule, which may be obtained by inverting all the stereogenic centers of the reference molecule. For example, if the reference molecule has S absolute stereochemical configuration, then the opposite enantiomer has R absolute stereochemical configuration. Likewise, if the reference molecule has S,S absolute stereochemical configuration, then the opposite enantiomer has R,R stereochemical configuration, and so on.

“Stereoisomer” and “stereoisomers” of a compound with given stereochemical configuration refer to the opposite enantiomer of the compound and to any diastereoisomers, including geometrical isomers (Z/E) of the compound. For example, if a compound has S,R,Z stereochemical configuration, its stereoisomers would include its opposite enantiomer having R,S,Z configuration, and its diastereomers having S,S,Z configuration, R,R,Z configuration, S,R,E configuration, R,S,E configuration, S,S,E configuration, and R,R,E configuration. If the stereochemical configuration of a compound is not specified, then “stereoisomer” refers to any one of the possible stereochemical configurations of the compound.

“Substantially pure stereoisomer” and variants thereof refer to a sample containing a compound having a specific stereochemical configuration and which comprises at least about 95% of the sample.

“Pure stereoisomer” and variants thereof refer to a sample containing a compound having a specific stereochemical configuration and which comprises at least about 99.5% of the sample.

“Subject” refers to a mammal, including a human.

“Pharmaceutically acceptable” substances refer to those substances which are suitable for administration to subjects.

“Treating” refers to reversing, alleviating, inhibiting the progress of, or preventing a disease, disorder or condition to which such term applies, or to reversing, alleviating, inhibiting the progress of, or preventing one or more symptoms of such disease, disorder or condition.

“Treatment” refers to the act of “treating,” as defined immediately above.

“Drug,” “drug substance,” “active pharmaceutical ingredient,” and the like, refer to a compound (e.g., compounds of Formula 1, including subgeneric compounds and compounds specifically named in the specification) that may be used for treating a subject in need of treatment.

“Effective amount” of a drug, “therapeutically effective amount” of a drug, and the like, refer to the quantity of the drug that may be used for treating a subject and may depend on the weight and age of the subject and the route of administration, among other things.

“Excipient” refers to any diluent or vehicle for a drug.

“Pharmaceutical composition” refers to the combination of one or more drug substances and one or more excipients.

“Drug product,” “pharmaceutical dosage form,” “dosage form,” “final dosage form” and the like, refer to a pharmaceutical composition suitable for treating a subject in need of treatment and generally may be in the form of tablets, capsules, sachets containing powder or granules, liquid solutions or suspensions, patches, films, and the like.

“Condition associated with SSTR4” and similar phrases relate to a disease, disorder or condition in a subject for which activation of SSTR4 may provide a therapeutic or prophylactic benefit.

The following abbreviations may be used in the specification: Ac (acetyl); ACN (acetonitrile); AIBN (azo-bis-isobutyronitrile); API (active pharmaceutical ingredient); aq (aqueous); BINAP (2,2′-bis(diphenylphosphino)-1,1′-binaphthyl); Boc (tert-butoxycarbonyl); Cbz (carbobenzyloxy); dba (dibenzylideneacetone); DBU (1,8-diazabicyclo[5.4.0]undec-7-ene); DCC (1,3-dicyclohexylcarbodiimide); DCE (1,1-dichloroethane); DCM (dichloromethane); DEA (diethylamine); DIAD (diisopropyl azodicarboxylate); DIPEA (N,N-diisopropylethylamine, Hünig's Base); DMA (N,N-dimethylacetamide); DMAP (4-dimethylaminopyridine); DME (1,2-dimethoxyethane); DMF (N,N-dimethylformamide); DMP (Dess-Martin periodinane); DMSO (dimethylsulfoxide); dppf (1,1′-bis(diphenylphosphino)ferrocene); DTT (dithiothreitol); EC₅₀ (effective concentration at half maximal response); EDA (ethoxylated dodecyl alcohol, Brj®35); EDC (N-(3-dimethylaminopropyl)-N′-ethylcarbodiimide); EDTA (ethylenediaminetetraacetic acid); ee (enantiomeric excess); eq (equivalents); Et (ethyl); Et₃N (triethylamine); EtOAc (ethyl acetate); EtOH (ethanol); HATU (2-(3H-[1,2,3]triazolo[4,5-b]pyridin-3-yl)-1,1,3,3-tetramethyluronium hexafluorophosphate(V)); HEPES (4-(2-hydroxyethyl)piperazine-1-ethanesulfonic acid); AcOH (acetic acid); HOBt (1H-benzo[d][1,2,3]triazol-1-ol); IC₅₀ (concentration at 50% inhibition); IPA (isopropanol); IPAc (isopropyl acetate); IPE (isopropylether); LDA (lithium diisopropylamide); LiHMDS (lithium bis(trimethylsilyl)amide); mCPBA (m-chloroperoxybenzoic acid); Me (methyl); MeOH (methanol); MTBE (methyl tert-butyl ether); mp (melting point); NaOt-Bu (sodium tertiary butoxide); NMM (N-methylmorpholine); NMP (N-methyl-pyrrolidone); OTf (triflate); PE (petroleum ether); Ph (phenyl); pEC₅₀ (−log₁₀ (EC₅₀), where EC₅₀ is given in molar (M) units); pIC₅₀ (−log₁₀ (IC₅₀), where IC₅₀ is given in molar (M) units); Pr (propyl); c-Pr (cyclopropyl), i-Pr (isopropyl); PTFE (polytetrafluoroethylene); PyBOP ((benzotriazol-1-yloxy)tripyrrolidinophosphonium hexafluorophosphate); PyBroP® (bromotripyrrolidino-phosphonium hexafluorophosphate); PCy₃ (tricyclohexylphosphine); RT (room temperature, approximately 20° C. to 25° C.); SFC (supercritical fluid chromatography); T3P (2,4,6-tripropyl-1,3,5,2,4,6-trioxatriphosphinane 2,4,6-trioxide); TCEP (tris(2-carboxyethyl)phos-phine); TFA (trifluoroacetic acid); TFAA (2,2,2-trifluoroacetic anhydride); THE (tetrahydrofuran); TMS (trimethylsilyl); and Tris buffer (2-amino-2-hydroxymethyl-propane-1,3-diol buffer).

As described, below, this disclosure concerns compounds of Formula 1 and their pharmaceutically acceptable salts. This disclosure also concerns materials and methods for preparing compounds of Formula 1, pharmaceutical compositions which contain them, and the use of compounds of Formula 1 and their pharmaceutically acceptable salts (optionally in combination with other pharmacologically active agents) for treating diseases, disorders or conditions of the CNS, including Alzheimer's disease, and to other diseases, disorders or conditions associated with SSTR4, including pain.

The compounds of Formula 1 include those in which:
(1) X¹ is selected from N and CR¹;
   n is selected from 0 and 1;
   R¹, R², R³, R⁴ and R⁵ are each independently selected from
      (i) hydrogen, halo, hydroxy and cyano; and
      (ii) C₁₋₃ alkyl, C₁₋₃ alkoxy and C₃₋₆ cycloalkyl, each unsubstituted or substituted with 1 to 3 substituents independently selected from halo;
   L⁶ is selected from —CH₂—, —N(R⁶)—, *—N(R⁶)CH₂— and —O—, wherein R⁶ is selected from hydrogen and C₁₋₃ alkyl, and * represents the point of attachment to an aromatic ring carbon atom;
   R⁷ and R⁸ are each independently selected from halo and C₁₋₃ alkyl, provided R⁷ and R⁸ are not both methyl; or R⁷ and R⁸, together with the carbon atom to which they are attached, form a cyclopropylidene;
   R⁹ and R¹⁰ are each independently selected from
      (i) hydrogen and halo; and
      (ii) C₁₋₃ alkyl and phenyl, each unsubstituted or substituted with 1 to 3 substituents independently selected from halo; or
   R⁹ and R¹⁰, together with the carbon atom to which they are attached, form a cyclopropylidene;
   R¹¹ and R¹² are each independently selected from hydrogen, halo and C₁₋₃ alkyl which is unsubstituted or substituted with 1 to 3 substituents independently selected from halo, provided no more than one of R¹¹ and R¹² is methyl; and
   (a) R¹³ is selected from hydrogen and C₁₋₃ alkyl; and
      R¹⁴ and R¹⁵ are each independently selected from hydrogen, halo, and C₁₋₃ alkyl which is unsubstituted or substituted with 1 to 3 substituents independently selected from halo, provided no more than one of R¹⁴ and R¹⁵ is methyl; or
   (b) R¹³ and R¹⁴ together form a propane-1,3-diyl bridging the nitrogen and carbon atoms to which they are respectively attached; and
      R¹⁵ is selected from hydrogen, halo, and C₁₋₃ alkyl which is unsubstituted or substituted with 1 to 3 substituents independently selected from halo;
   provided
      (i) if X¹ is CR¹, L⁶ is —CH₂—, and R⁷ and R⁸ form a cyclopropylidene, then at least one of R¹, R², R³, R⁴, R⁵, R⁹, R¹⁰, R¹¹, R¹², R¹³, R¹⁴ and R¹⁵ is not hydrogen;
      (ii) if X¹ is CR¹, L⁶ is —CH₂—, n is 0, R¹, R², R⁴, R⁵, R¹¹, R¹², R¹⁴ and R¹⁵ are each hydrogen, R⁷ and R⁸ form a cyclopropylidene, and R⁹, R¹⁰ and R¹³ are each methyl, then R³ is not fluoro or hydroxy;
      (iii) if X¹ is CR¹, L⁶ is —CH₂—, n is 0, R¹ is fluoro, R², R³, R⁴, R⁵, R¹¹, R¹², R¹⁴ and R¹⁵ are each hydrogen, and R⁷ and R⁸ form a cyclopropylidene, then R⁹, R¹⁰ and R¹³ are not all hydrogen and not all methyl;
      (iv) if X¹ is CR¹, L⁶ is —CH₂—, n is 1, R¹, R², R³, R⁴, R⁵, R¹⁰, R¹¹, R¹², R¹³, R¹⁴ and R¹⁵ are each hydrogen, and R⁷ and R⁸ form a cyclopropylidene, then R⁹ is not methyl;
      (v) if X¹ is CR¹, L⁶ is —CH₂—, n is 1, R¹, R², R³, R⁴, R⁵, R⁹, R¹⁰, R¹², R¹³, R¹⁴ and R¹⁵ are each hydrogen, and R⁷ and R⁸ form a cyclopropylidene, then R¹¹ is not methyl;
      (vi) if X¹ is CR¹, n is 0, R¹, R², R³, R⁴, R⁵, R¹¹, R¹², R¹⁴ and R¹⁵ are each hydrogen, R⁷ and R⁸ form a cyclopropylidene, R⁹ and R¹⁰ are each methyl, and R¹³ is ethyl, then L⁶ is not —O— or —NH—;
      (vii) if X¹ is CR¹, L⁶ is —CH₂—, n is 0, R¹, R², R⁴, R⁵, R¹¹, R¹², R¹⁴ and R¹⁵ are each hydrogen, R⁷ and R⁸ form a cyclopropylidene, R⁹ and R¹⁰ are both methyl, and R¹³ is ethyl, then R³ is not hydroxy;
      (viii) if X¹ is CR¹, L⁶ is —CH₂—, n is 1, R⁷ and R⁸ form a cyclopropylidene, and R¹³ and R¹⁴ together form a propane-1,3-diyl bridging the nitrogen and carbon atoms to which they are respectively attached, then at least one of R¹, R², R³, R⁴, R⁵, R⁹, R¹⁰, R¹¹, R¹² and R¹⁵ is not hydrogen;
      (ix) if X¹ is CR¹, L⁶ is —CH₂—, n is 1, R⁷ and R⁸ form a cyclopropylidene, and R¹³ and R¹⁴ together form a propane-1,3-diyl bridging the nitrogen and carbon atoms to which they are respectively attached, and R², R³, R⁴, R⁵, R⁹, R¹⁰, R¹¹, R¹² and R¹⁵ are each hydrogen, then R¹ is not fluoro; and
      (x) if X¹ is CR¹, L⁶ is —CH₂—, n is 1, R⁷ and R⁸ form a cyclopropylidene, and R¹³ and R¹⁴ together form a propane-1,3-diyl bridging the nitrogen and carbon atoms to which they are respectively attached, and R¹, R², R⁴, R⁵, R⁹, R¹⁰, R¹¹, R¹² and R¹⁵ are each hydrogen, then R³ is not fluoro.

In addition to embodiment (1) in the preceding paragraph, the compounds of Formula 1 include those in which:
(2) X¹ is CR¹.

In addition to embodiment (2) in the preceding paragraph, the compounds of Formula 1 include those in which R¹ is selected from:
(3) (i) hydrogen, halo and cyano; and
   (ii) C₁₋₃ alkyl, C₁₋₃ alkoxy and C₃₋₆ cycloalkyl, each unsubstituted or substituted with 1 to 3 substituents independently selected from halo;
(4) (i) hydrogen, halo and cyano; and
   (ii) C₁₋₃ alkyl, C₁₋₃ alkoxy and C₃₋₆ cycloalkyl, each unsubstituted;
(5) (i) hydrogen, chloro, fluoro and cyano; and
   (ii) C₁₋₃ alkyl, C₁₋₃ alkoxy and C₃₋₆ cycloalkyl, each unsubstituted or substituted with 1 to 3 substituents independently selected from halo;
(6) (i) hydrogen, chloro, fluoro and cyano; and
   (ii) C₁₋₃ alkyl, C₁₋₃ alkoxy and C₃₋₆ cycloalkyl, each unsubstituted or substituted with 1 to 3 fluoro;
(7) (i) hydrogen, chloro, fluoro and cyano; and
   (ii) C₁₋₃ alkyl, C₁₋₃ alkoxy and C₃₋₆ cycloalkyl, each unsubstituted;
(8) (i) hydrogen, halo and cyano; and
   (ii) methyl, methoxy and cyclopropyl, each unsubstituted or substituted with 1 to 3 substituents independently selected from halo;
(9) (i) hydrogen, halo and cyano; and
   (ii) methyl, methoxy and cyclopropyl, each unsubstituted or substituted with 1 to 3 fluoro;
(10) hydrogen, halo, cyano, methyl, methoxy and cyclopropyl;
(11) hydrogen, chloro, fluoro, cyano, methyl, methoxy and cyclopropyl;
(12) hydrogen, chloro, fluoro, cyano, methyl and methoxy;
(13) hydrogen, chloro and methyl; or
(14) hydrogen.

In addition to embodiment (1) above, the compounds of Formula 1 include those in which:
(15) X¹ is N.

In addition to embodiments (1) to (15) in the preceding paragraphs, the compounds of Formula 1 include those in which R² is selected from:
(16) (i) hydrogen and halo; and
   (ii) C₁₋₃ alkyl, C₁₋₃ alkoxy and C₃₋₆ cycloalkyl, each unsubstituted or substituted with 1 to 3 substituents independently selected from halo;
(17) (i) hydrogen and halo; and
   (ii) C₁₋₃ alkyl, C₁₋₃ alkoxy and C₃₋₆ cycloalkyl, each unsubstituted;
(18) (i) hydrogen, chloro and fluoro; and
   (ii) C₁₋₃ alkyl, C₁₋₃ alkoxy and C₃₋₆ cycloalkyl, each unsubstituted or substituted with 1 to 3 substituents independently selected from halo;
(19) (i) hydrogen, chloro and fluoro; and
   (ii) C₁₋₃ alkyl, C₁₋₃ alkoxy and C₃₋₆ cycloalkyl, each unsubstituted or substituted with 1 to 3 fluoro;
(20) (i) hydrogen, chloro and fluoro; and
   (ii) C₁₋₃ alkyl, C₁₋₃ alkoxy and C₃₋₆ cycloalkyl, each unsubstituted;
(21) (i) hydrogen and halo; and
   (ii) C₁₋₃ alkyl and C₁₋₃ alkoxy, each unsubstituted or substituted with 1 to 3 substituents independently selected from halo;
(22) (i) hydrogen and halo; and
   (ii) methyl and methoxy, each unsubstituted or substituted with 1 to 3 substituents independently selected from halo;
(23) (i) hydrogen and halo; and
   (ii) methyl and methoxy, each unsubstituted or substituted with 1 to 3 fluoro;
(24) hydrogen, halo, methyl and methoxy;
(25) hydrogen, chloro, fluoro and methyl; or
(26) hydrogen, chloro and methyl.

In addition to embodiments (1) to (26) in the preceding paragraphs, the compounds of Formula 1 include those in which R³ is selected from:
(27) (i) hydrogen and halo; and
   (ii) C₁₋₃ alkyl, C₁₋₃ alkoxy and C₃₋₆ cycloalkyl, each unsubstituted or substituted with 1 to 3 substituents independently selected from halo;
(28) (i) hydrogen and halo; and
   (ii) C₁₋₃ alkyl, C₁₋₃ alkoxy and C₃₋₆ cycloalkyl, each unsubstituted;
(29) (i) hydrogen, chloro and fluoro; and
   (ii) C₁₋₃ alkyl, C₁₋₃ alkoxy and C₃₋₆ cycloalkyl, each unsubstituted or substituted with 1 to 3 substituents independently selected from halo;
(30) (i) hydrogen, chloro and fluoro; and
   (ii) C₁₋₃ alkyl, C₁₋₃ alkoxy and C₃₋₆ cycloalkyl, each unsubstituted or substituted with 1 to 3 fluoro;
(31) (i) hydrogen, chloro and fluoro; and
   (ii) C₁₋₃ alkyl, C₁₋₃ alkoxy and C₃₋₆ cycloalkyl, each unsubstituted;
(32) (i) hydrogen and halo; and
   (ii) C₁₋₃ alkyl which is unsubstituted or substituted with 1 to 3 substituents independently selected from halo;
(33) (i) hydrogen and halo; and
   (ii) methyl which is unsubstituted or substituted with 1 to 3 substituents independently selected from halo;
(34) (i) hydrogen and halo; and
   (ii) methyl which is unsubstituted or substituted with 1 to 3 fluoro;
(35) hydrogen, halo and methyl;
(36) hydrogen, chloro, fluoro and methyl; or
(37) hydrogen and chloro.

In addition to embodiments (1) to (37) in the preceding paragraphs, the compounds of Formula 1 include those in which R⁴ is selected from:
(38) (i) hydrogen and halo; and
   (ii) C₁₋₃ alkyl, C₁₋₃ alkoxy and C₃₋₆ cycloalkyl, each unsubstituted or substituted with 1 to 3 substituents independently selected from halo;
(39) (i) hydrogen and halo; and
   (ii) C₁₋₃ alkyl, C₁₋₃ alkoxy and C₃₋₆ cycloalkyl, each unsubstituted;
(40) (i) hydrogen, chloro and fluoro; and
   (ii) C₁₋₃ alkyl, C₁₋₃ alkoxy and C₃₋₆ cycloalkyl, each unsubstituted or substituted with 1 to 3 substituents independently selected from halo;
(41) (i) hydrogen, chloro and fluoro; and
   (ii) C₁₋₃ alkyl, C₁₋₃ alkoxy and C₃₋₆ cycloalkyl, each unsubstituted or substituted with 1 to 3 fluoro;
(42) (i) hydrogen, chloro and fluoro; and
   (ii) C₁₋₃ alkyl, C₁₋₃ alkoxy and C₃₋₆ cycloalkyl, each unsubstituted;
(43) (i) hydrogen and halo; and
   (ii) C₁₋₃ alkyl and C₁₋₃ alkoxy, each unsubstituted or substituted with 1 to 3 substituents independently selected from halo;
(44) (i) hydrogen and halo; and
   (ii) methyl and methoxy, each unsubstituted or substituted with 1 to 3 substituents independently selected from halo;
(45) (i) hydrogen and halo; and
   (ii) methyl and methoxy, each unsubstituted or substituted with 1 to 3 fluoro;
(46) hydrogen, halo, methyl and methoxy;
(47) hydrogen, chloro, fluoro and methyl;
(48) hydrogen, chloro and fluoro;
(49) hydrogen and fluoro; or
(50) hydrogen.

In addition to embodiments (1) to (50) in the preceding paragraphs, the compounds of Formula 1 include those in which R⁵ is selected from:
(51) (i) hydrogen, halo and cyano; and
   (ii) C₁₋₃ alkyl, C₁₋₃ alkoxy and C₃₋₆ cycloalkyl, each unsubstituted or substituted with 1 to 3 substituents independently selected from halo;
(52) (i) hydrogen, halo and cyano; and
   (ii) C₁₋₃ alkyl, C₁₋₃ alkoxy and C₃₋₆ cycloalkyl, each unsubstituted;
(53) (i) hydrogen, chloro, fluoro and cyano; and
   (ii) C₁₋₃ alkyl, C₁₋₃ alkoxy and C₃₋₆ cycloalkyl, each unsubstituted or substituted with 1 to 3 substituents independently selected from halo;
(54) (i) hydrogen, chloro, fluoro and cyano; and
   (ii) C₁₋₃ alkyl, C₁₋₃ alkoxy and C₃₋₆ cycloalkyl, each unsubstituted or substituted with 1 to 3 fluoro;
(55) (i) hydrogen, chloro, fluoro and cyano; and
   (ii) C₁₋₃ alkyl, C₁₋₃ alkoxy and C₃₋₆ cycloalkyl, each unsubstituted;
(56) (i) hydrogen, halo and cyano; and
   (ii) methyl, methoxy and cyclopropyl, each unsubstituted or substituted with 1 to 3 substituents independently selected from halo;
(57) (i) hydrogen, halo and cyano; and
   (ii) methyl, methoxy and cyclopropyl, each unsubstituted or substituted with 1 to 3 fluoro;
(58) hydrogen, halo, cyano, methyl, methoxy and cyclopropyl;
(59) hydrogen, chloro, fluoro, cyano, methyl, methoxy and cyclopropyl;
(60) hydrogen, chloro, fluoro, cyano, methyl and methoxy;
(61) hydrogen, chloro and methyl; or
(62) hydrogen.

In addition to embodiments (1) to (62) in the preceding paragraphs, the compounds of Formula 1 include those in which L⁶ is selected from:
(63) —CH₂— and —O—;
(64) —CH₂—;
(65) —O—;
(66) —N(R⁶)— and *—N(R⁶)CH₂—; or
(67) *—N(R⁶)CH₂—.

In addition to embodiments (66) to (67) in the preceding paragraph, the compounds of Formula 1 include those in which R⁶ is selected from:
(68) hydrogen and methyl;
(69) methyl; or
(70) hydrogen.

In addition to embodiments (1) to (70) in the preceding paragraphs, the compounds of Formula 1 include those in which R⁷ and R⁸ are each independently selected from:
(71) halo and C₁₋₃ alkyl, provided R⁷ and R⁸ are not both methyl;
(72) fluoro and C₁₋₃ alkyl, provided R⁷ and R⁸ are not both methyl;
(73) halo and methyl, provided R⁷ and R⁸ are not both methyl;
(74) fluoro and methyl, provided R⁷ and R⁸ are not both methyl;
(75) halo; or
(76) fluoro.

In addition to embodiments (1) to (70) in the preceding paragraphs, the compounds of Formula 1 include those in which:
(77) R⁷ and R⁸, together with the carbon atom to which they are attached, form a cyclopropylidene.

In addition to embodiments (1) to (77) in the preceding paragraphs, the compounds of Formula 1 include those in which R⁹ and R¹⁰ are each independently selected from:
(78) (i) hydrogen and halo; and
   (ii) C₁₋₃ alkyl and phenyl, each unsubstituted or substituted with 1 to 3 substituents independently selected from halo;
(79) (i) hydrogen and halo; and
   (ii) C₁₋₃ alkyl and phenyl, each unsubstituted;
(80) (i) hydrogen and fluoro; and
   (ii) C₁₋₃ alkyl and phenyl, each unsubstituted or substituted with 1 to 3 substituents independently selected from halo;
(81) (i) hydrogen and fluoro; and
   (ii) C₁₋₃ alkyl and phenyl, each unsubstituted or substituted with 1 to 3 substituents independently selected from fluoro;
(82) (i) hydrogen and fluoro; and
   (ii) C₁₋₃ alkyl and phenyl, each unsubstituted;
(83) (i) hydrogen and halo; and
   (ii) methyl, ethyl, isopropyl and phenyl, each unsubstituted or substituted with 1 to 3 substituents independently selected from halo;
(84) (i) hydrogen and halo; and
   (ii) methyl, ethyl, isopropyl and phenyl, each unsubstituted or substituted with 1 to 3 substituents independently selected from chloro and fluoro;
(85) (i) hydrogen and halo; and
   (ii) methyl, ethyl, isopropyl and phenyl, each unsubstituted or substituted with 1 to 3 fluoro;
(86) hydrogen, halo, methyl, ethyl, isopropyl and phenyl;
(87) hydrogen, fluoro, methyl, ethyl, isopropyl and phenyl; or
(88) hydrogen, fluoro, and methyl.

In addition to embodiments (78) to (88) in the preceding paragraph, the compounds of Formula 1 include those in which:
(89) at least one of R⁹ and R¹⁰ is hydrogen.

In addition to embodiments (1) to (77) in the preceding paragraphs, the compounds of Formula 1 include those in which:
(90) R⁹ and R¹⁰ are both hydrogen; or
(91) R⁹ and R¹⁰, together with the carbon atom to which they are attached, form a cyclopropylidene.

In addition to embodiments (1) to (91) in the preceding paragraphs, the compounds of Formula 1 include those in which R¹¹ and R¹² are each independently selected from:
(92) hydrogen, halo and C₁₋₃ alkyl which is unsubstituted;
(93) hydrogen, fluoro and C₁₋₃ alkyl which is unsubstituted or substituted with 1 to 3 substituents independently selected from halo;
(94) hydrogen, halo and methyl which is unsubstituted or substituted with 1 to 3 substituents independently selected from halo;
(95) hydrogen, halo and methyl which is unsubstituted or substituted with 1 to 3 fluoro;
(96) hydrogen, halo and methyl; or
(97) hydrogen, fluoro and methyl.

In addition to embodiments (92) to (97) in the preceding paragraph, the compounds of Formula 1 include those in:
(98) at least one of R¹¹ and R¹² is hydrogen.

In addition to embodiments (1) to (91) in the preceding paragraphs, the compounds of Formula 1 include those in which:
(99) R¹¹ and R¹² are both hydrogen.

In addition to embodiments (1) to (99) in the preceding paragraphs, the compounds of Formula 1 include those in which R¹³ is selected from:
(100) hydrogen and C₁₋₃ alkyl; or
(101) hydrogen and methyl.

In addition to embodiments (100) to (101) in the preceding paragraph, the compounds of Formula 1 include those in which no more than one of R¹⁴ and R¹⁵ is methyl and each R¹⁴ and R¹⁵ is independently selected from:
(102) hydrogen, halo, and C₁₋₃ alkyl which is unsubstituted or substituted with 1 to 3 substituents independently selected from halo;
(103) from hydrogen, halo, and C₁₋₃ alkyl which is unsubstituted;
(104) hydrogen, fluoro and C₁₋₃ alkyl which is unsubstituted or substituted with 1 to 3 substituents independently selected from halo;
(105) hydrogen, halo and methyl which is unsubstituted or substituted with 1 to 3 substituents independently selected from halo;
(106) hydrogen, halo and methyl which is unsubstituted or substituted with 1 to 3 fluoro;
(107) hydrogen, halo and methyl; or
(108) hydrogen, fluoro and methyl.

In addition to embodiments (102) to (108) in the preceding paragraph, the compounds of Formula 1 include those in which:
(109) at least one of R¹⁴ and R¹⁵ is hydrogen.

In addition to embodiments (100) to (101) above, the compounds of Formula 1 include those in which:
(110) R¹⁴ and R¹⁵ are both hydrogen.

In addition to embodiments (1) to (99) in the preceding paragraphs, the compounds of Formula 1 include those in which:
(111) R¹³ and R¹⁴ together form a propane-1,3-diyl bridging the nitrogen and carbon atoms to which they are respectively attached.

In addition to embodiment (111) in the preceding paragraph, the compounds of Formula 1 include those in which R¹⁵ is selected from:
(112) hydrogen, halo, and C₁₋₃ alkyl which is unsubstituted or substituted with 1 to 3 substituents independently selected from halo;
(113) hydrogen, halo, and C₁₋₃ alkyl which is unsubstituted;
(114) hydrogen, fluoro and C₁₋₃ alkyl which is unsubstituted or substituted with 1 to 3 substituents independently selected from halo;
(115) hydrogen, halo and methyl which is unsubstituted or substituted with 1 to 3 substituents independently selected from halo;
(116) hydrogen, halo and methyl which is unsubstituted or substituted with 1 to 3 fluoro;
(117) hydrogen, halo and methyl; or
(118) hydrogen, fluoro and methyl.

In addition to embodiments (111) to (118) in the preceding paragraphs, the compounds of Formula 1 include those in which R¹⁵ is:
(119) hydrogen.

In addition to embodiments (1) to (119) in the preceding paragraphs, the compounds of Formula 1 include those in which n is:
(120) 0; or
(121) 1.

Compounds of Formula 1 for use as a medicament may include those in which:
(122) X¹ is selected from N and CR¹;
   n is selected from 0 and 1;
   R¹, R², R³, R⁴ and R⁵ are each independently selected from
      (i) hydrogen, halo, hydroxy and cyano; and
      (ii) C₁₋₃ alkyl, C₁₋₃ alkoxy and C₃₋₆ cycloalkyl, each unsubstituted or substituted with 1 to 3 substituents independently selected from halo;
   L⁶ is selected from —CH₂—, —N(R⁶)—, *—N(R⁶)CH₂— and —O—, wherein R⁶ is selected from hydrogen and C₁₋₃ alkyl, and * represents the point of attachment to an aromatic ring carbon atom;
   R⁷ and R⁸ are each independently selected from hydrogen, halo and C₁₋₃ alkyl, wherein at least one of R⁷ and R⁸ is not hydrogen; or R⁷ and R⁸, together with the carbon atom to which they are attached, form a C₃₋₆ cycloalkylidene;
   R⁹ and R¹⁰ are each independently selected from
      (i) hydrogen and halo; and
      (ii) C₁₋₃ alkyl and phenyl, each unsubstituted or substituted with 1 to 3 substituents independently selected from halo; or
   R⁹ and R¹⁰, together with the carbon atom to which they are attached, form a cyclopropylidene;
   R¹¹ and R¹² are each independently selected from hydrogen, halo and C₁₋₃ alkyl which is unsubstituted or substituted with 1 to 3 substituents independently selected from halo, provided no more than one of R¹¹ and R¹² is methyl; and
   (a) R¹³ is selected from hydrogen and C₁₋₃ alkyl; and
      R¹⁴ and R¹⁵ are each independently selected from hydrogen, halo, and C₁₋₃ alkyl which is unsubstituted or substituted with 1 to 3 substituents independently selected from halo, provided no more than one of R¹⁴ and R¹⁵ is methyl; or
   (b) R¹³ and R¹⁴ together form a propane-1,3-diyl bridging the nitrogen and carbon atoms to which they are respectively attached; and
      R¹⁵ is selected from hydrogen, halo, and C₁₋₃ alkyl which is unsubstituted or substituted with 1 to 3 substituents independently selected from halo;
   provided
      (i) if X is CR¹, n is 1, L⁶ is —O—, R⁷ is methyl and R⁸ is hydrogen, then at least one of R¹, R², R³, R⁴, R⁵, R⁹, R¹⁰, R¹¹, R¹², R¹³, R¹⁴ or R¹⁵ is not hydrogen;
      (ii) if X is CR¹, n is 1, L⁶ is —O—, R⁷ and R⁸ are both methyl, and R¹, R², R⁴, R⁵, R⁹, R¹⁰, R¹¹, R¹², R¹³, R¹⁴ and R¹⁵ are each hydrogen, then R³ is not chloro; and
      (iii) if X is CR¹, n is 1, L⁶ is —O—, R⁷ is methyl or ethyl, R¹, R², R³, R⁴, R⁵, R⁸, R¹⁰, R¹¹, R¹², R¹³, R¹⁴ and R¹⁵ are each hydrogen, then R⁹ is not unsubstituted phenyl.

Compounds of Formula 1 include embodiments (1) through (122) described in the preceding paragraphs and compounds specifically named in the examples, may exist as salts, complexes, solvates, hydrates, and liquid crystals. Likewise, compounds of Formula 1 that are salts may exist as complexes, solvates, hydrates, and liquid crystals.

Compounds of Formula 1 may form pharmaceutically acceptable complexes, salts, solvates and hydrates. These salts include acid addition salts (including di-acids) and base salts. Pharmaceutically acceptable acid addition salts include salts derived from inorganic acids such as hydrochloric acid, nitric acid, phosphoric acid, sulfuric acid, hydrobromic acid, hydroiodic acid, hydrofluoric acid, and phosphorous acids, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include salts derived from bases, including metal cations, such as an alkali or alkaline earth metal cation, as well as amines. Examples of suitable metal cations include sodium, potassium, magnesium, calcium, zinc, and aluminum. Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2002).

Pharmaceutically acceptable salts may be prepared using various methods. For example, a compound of Formula 1 may be reacted with an appropriate acid or base to give the desired salt. Alternatively, a precursor of the compound of Formula 1 may be reacted with an acid or base to remove an acid- or base-labile protecting group or to open a lactone or lactam group of the precursor. Additionally, a salt of the compound of Formula 1 may be converted to another salt (or free form) through treatment with an appropriate acid or base or through contact with an ion exchange resin. Following reaction, the salt may be isolated by filtration if it precipitates from solution, or by evaporation to recover the salt. The degree of ionization of the salt may vary from completely ionized to almost non-ionized.

Compounds of Formula 1 may exist in a continuum of solid states ranging from fully amorphous to fully crystalline. The term “amorphous” refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”). The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (“melting point”).

Compounds of Formula 1 may also exist in unsolvated and solvated forms. The term “solvate” describes a molecular complex comprising the compound and one or more pharmaceutically acceptable solvent molecules (e.g., ethanol). The term “hydrate” is a solvate in which the solvent is water. Pharmaceutically acceptable solvates include those in which the solvent may be isotopically substituted (e.g., D₂O, acetone-d₆, DMSO-d₆).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will typically be observed.

Compounds of Formula 1 may also exist as multi-component complexes (other than salts and solvates) in which the compound (drug) and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun. (2004) 17:1889-1896. For a general review of multi-component complexes, see J. K. Haleblian, J. Pharm. Sci. (1975) 64(8):1269-88.

When subjected to suitable conditions, compounds of Formula 1 may exist in a mesomorphic state (mesophase or liquid crystal). The mesomorphic state lies between the true crystalline state and the true liquid state (either melt or solution). Mesomorphism arising as the result of a change in temperature is described as “thermotropic” and mesomorphism resulting from the addition of a second component, such as water or another solvent, is described as “lyotropic.” Compounds that have the potential to form lyotropic mesophases are described as “amphiphilic” and include molecules which possess a polar ionic moiety (e.g., —COO⁻Na⁺, —COO⁻K⁺, —SO₃⁻Na⁺) or polar non-ionic moiety (such as —N⁻N⁺ (CH₃)₃). See, e.g., N. H. Hartshorne and A. Stuart, Crystals and the Polarizing Microscope (4th ed, 1970).

Each compound of Formula 1 may exist as polymorphs, stereoisomers, tautomers, or some combination thereof, may be isotopically-labeled, may result from the administration of a prodrug, or form a metabolite following administration.

“Prodrugs” refer to compounds having little or no pharmacological activity that can, when metabolized in vivo, undergo conversion to compounds having desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of compounds of Formula 1 having carboxylic acid, hydroxy, or amino functional groups, respectively. For further discussions of prodrugs, see e.g., T. Higuchi and V. Stella “Pro-drugs as Novel Delivery Systems,” ACS Symposium Series 14 (1975) and E. B. Roche ed., Bioreversible Carriers in Drug Design (1987).

“Metabolites” refer to compounds formed in vivo upon administration of pharmacologically active compounds. Examples include hydroxymethyl, hydroxy, secondary amino, primary amino, phenol, and carboxylic acid derivatives of compounds of Formula 1 having methyl, alkoxy, tertiary amino, secondary amino, phenyl, and amide groups, respectively.

Compounds of Formula 1 may exist as stereoisomers that result from the presence of one or more stereogenic centers, one or more double bonds, or both. The stereoisomers may be pure, substantially pure, or mixtures. Such stereoisomers may also result from acid addition or base salts in which the counter-ion is optically active, for example, when the counter-ion is D-lactate or L-lysine.

Compounds of Formula 1 may exist as tautomers, which are isomers resulting from tautomerization. Tautomeric isomerism includes, for example, imine-enamine, keto-enol, oxime-nitroso, and amide-imidic acid tautomerism.

Compounds of Formula 1 may exhibit more than one type of isomerism.

Geometrical (cis/trans) isomers may be separated by conventional techniques such as chromatography and fractional crystallization.

Conventional techniques for preparing or isolating a compound having a specific stereochemical configuration include chiral synthesis from a suitable optically pure precursor or resolution of the racemate (or the racemate of a salt or derivative) using, for example, chiral high-pressure liquid chromatography (HPLC). Alternatively, the racemate (or a racemic precursor) may be reacted with a suitable optically active compound, for example, an alcohol, or, in the case where the compound of Formula 1 contains an acidic or basic moiety, an acid or base such as tartaric acid or 1-phenylethylamine. The resulting diastereomeric mixture may be separated by chromatography, fractional crystallization, etc., and the appropriate diastereoisomer converted to the compound having the requisite stereochemical configuration. For a further discussion of techniques for separating stereoisomers, see E. L. Eliel and S. H. Wilen, Stereochemistry of Organic Compounds (1994).

Compounds of Formula 1 may possess isotopic variations, in which at least one atom is replaced by an atom having the same atomic number, but an atomic mass different from the atomic mass usually found in nature. Isotopes suitable for inclusion in compounds of Formula 1 include, for example, isotopes of hydrogen, such as ²H and ³H; isotopes of carbon, such as ¹¹C, ¹³C and ¹⁴C; isotopes of nitrogen, such as ¹³N and ¹⁵N; isotopes of oxygen, such as ¹⁵0, ¹⁷0 and ¹⁸O; isotopes of sulfur, such as ³⁵S; isotopes of fluorine, such as 18F; isotopes of chlorine, such as ³⁶Cl, and isotopes of iodine, such as ¹²¹3 and ¹²⁵. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies for examining substrate receptor occupancy. Isotopically-labeled compounds may be prepared by processes analogous to those described elsewhere in the disclosure using an appropriate isotopically-labeled reagent in place of a non-labeled reagent.

The compounds of Formula 1 may be prepared using the techniques described below. Some of the methods and examples may omit details of common reactions, including oxidations, reductions, and so on, separation techniques (extraction, evaporation, precipitation, chromatography, filtration, trituration, crystallization, and the like), and analytical procedures, which are known to persons of ordinary skill in the art of organic chemistry. The details of such reactions and techniques can be found in several treatises, including Richard Larock, Comprehensive Organic Transformations (1999), and the multi-volume series edited by Michael B. Smith and others, Compendium of Organic Synthetic Methods (1974 et seq.). Starting materials and reagents may be obtained from commercial sources or may be prepared using literature methods. Some of the reaction schemes may omit minor products resulting from chemical transformations (e.g., an alcohol from the hydrolysis of an ester, CO₂ from the decarboxylation of a di-acid, etc.). In addition, in some instances, reaction intermediates may be used in subsequent steps without isolation or purification (i.e., in situ).

In the methods and examples below, certain compounds may be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and so on, see T. W. Greene and P. G. Wuts, Protecting Groups in Organic Chemistry (1999) and P. Kocienski, Protective Groups (2000).

Generally, the chemical transformations described throughout the specification may be carried out using substantially stoichiometric amounts of reactants, though certain reactions may benefit from using an excess of one or more of the reactants. Additionally, many of the reactions disclosed throughout the specification may be carried out at about room temperature (RT) and ambient pressure, but depending on reaction kinetics, yields, and so on, some reactions may be run at elevated pressures or employ higher temperatures (e.g., reflux conditions) or lower temperatures (e.g., −78° C. to 0° C.). Any reference in the disclosure and claims to a stoichiometric range, a temperature range, a pH range, etc., whether expressly using the word “range,” also includes the indicated endpoints.

Many of the chemical transformations may also employ one or more compatible solvents, which may influence the reaction rate and yield. Depending on the nature of the reactants, the one or more solvents may be polar protic solvents (including water), polar aprotic solvents, non-polar solvents, or some combination. Representative solvents include saturated aliphatic hydrocarbons (e.g., n-pentane, n-hexane, n-heptane, n-octane, cyclohexane, methylcyclohexane); aromatic hydrocarbons (e.g., benzene, toluene, xylenes); halogenated hydrocarbons (e.g., methylene chloride, chloroform, carbon tetrachloride); aliphatic alcohols (e.g., methanol, ethanol, propan-1-ol, propan-2-ol, butan-1-ol, 2-methyl-propan-1-ol, butan-2-ol, 2-methyl-propan-2-ol, pentan-1-ol, 3-methyl-butan-1-ol, hexan-1-ol, 2-methoxy-ethanol, 2-ethoxy-ethanol, 2-butoxy-ethanol, 2-(2-methoxy-ethoxy)-ethanol, 2-(2-ethoxy-ethoxy)-ethanol, 2-(2-butoxy-ethoxy)-ethanol); ethers (e.g., diethyl ether, di-isopropyl ether, dibutyl ether, 1,2-dimethoxy-ethane, 1,2-diethoxy-ethane, 1-methoxy-2-(2-methoxy-ethoxy)-ethane, 1-ethoxy-2-(2-ethoxy-ethoxy)-ethane, tetrahydrofuran, 1,4-dioxane); ketones (e.g., acetone, methyl ethyl ketone); esters (methyl acetate, ethyl acetate); nitrogen-containing solvents (e.g., formamide, N,N-dimethylformamide, acetonitrile, N-methyl-pyrrolidone, pyridine, quinoline, nitrobenzene); sulfur-containing solvents (e.g., carbon disulfide, dimethyl sulfoxide, tetrahydro-thiophene-1,1,-dioxide); and phosphorus-containing solvents (e.g., hexamethylphosphoric triamide).

In the scheme, below, substituent identifiers (e.g., L⁶, n, R², R³, R⁴, R⁵, R⁷, R⁸, R⁹, R¹⁰, R¹¹, R¹², R¹³, R¹⁴ and R¹⁵) are as defined above for Formula 1. As mentioned earlier, some of the starting materials and intermediates may include protecting groups, which are removed prior to the final product. In such cases, the substituent identifier refers to moieties defined in Formula 1 and to those moieties with appropriate protecting groups. For example, a starting material or intermediate in the synthetic methods may include a potentially reactive (secondary) amine. In such cases, the amine would include the moiety with or without, say, a Boc or Cbz group attached to the amine.

Scheme A shows a general method for preparing compounds of Formula 1. In accordance with the method, an aryl or heteroaryl(alkyl, oxyalkyl or aminoalkyl)carboxylic acid (A1) is reacted with a primary amine (A2) or suitable base addition salt (e.g., lithium salt). The reaction is carried out using standard amide coupling agents, such as HATU, DCC, EDC hydrochloride, T3P or 2-chloro-1-methylpyridin-1-ium iodide, in the presence of a non-nucleophilic base (e.g., Et₃N, DIPEA) and one or more compatible solvents (e.g. ACN, DCM, DMA, DMF, NMP, pyridine, TIF). The amide coupling may be carried out at temperatures which range from room temperature to about 80° C. HOBt may be used to facilitate the reaction.

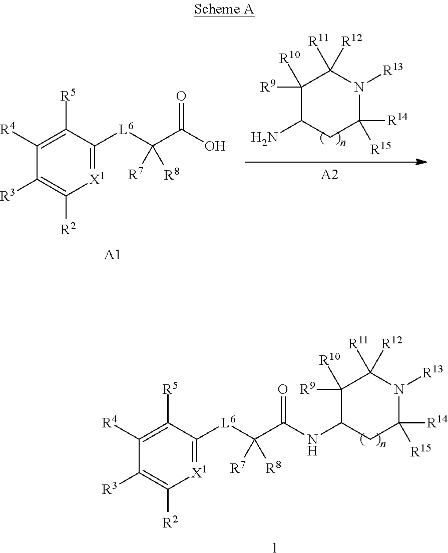

Though not shown in Scheme A, the starting materials may include a protected (e.g., Boc-substituted) secondary amine. In such cases, the amine is subsequently deprotected (e.g., by acid treatment) following amide coupling to reveal the secondary amine, which may be alkylated, e.g. via reaction with an alkyl halide (e.g., R¹³Y¹, where R¹³═C₁₋₃ alkyl and Y¹ ═Br, I) in the presence of a non-nucleophilic base (e.g., K₂CO₃) and compatible solvent (e.g., DMSO). Alternatively, the secondary amine may be reacted with an appropriate alkyl aldehyde under acidic conditions in the presence of a mild reducing agent, such as sodium cyanoborohydride or sodium acetoxyborohydride, and a compatible solvent (e.g. MeOH, DCM) to give requisite R¹³. The N-alkylation and reductive amination steps may be conducted at room temperature or above.

The methods depicted in the scheme may be varied as desired. For example, protecting groups may be added or removed and products may be further elaborated via, for example, alkylation, acylation, hydrolysis, oxidation, reduction, amidation, sulfonation, alkynation, and the like to give the desired final product. Furthermore, any intermediate or final product which comprises mixture of stereoisomers may be optionally purified by chiral column chromatography (e.g., supercritical fluid chromatography) or by derivatization with optically-pure reagents as described above to give a desired stereoisomer.

Compounds of Formula 1, which include compounds named above, and their pharmaceutically acceptable complexes, salts, solvates and hydrates, should be assessed for their biopharmaceutical properties, such as solubility and solution stability across pH, permeability, and the like, to select an appropriate dosage form and route of administration. Compounds that are intended for pharmaceutical use may be administered as crystalline or amorphous products, and may be obtained, for example, as solid plugs, powders, or films by methods such as precipitation, crystallization, freeze drying, spray drying, evaporative drying, microwave drying, or radio frequency drying.

Compounds of Formula 1 may be administered alone or in combination with one another or with one or more pharmacologically active compounds which are different than the compounds of Formula 1. Generally, one or more of these compounds are administered as a pharmaceutical composition (a formulation) in association with one or more pharmaceutically acceptable excipients. The choice of excipients depends on the mode of administration, the effect of the excipient on solubility and stability, and the nature of the dosage form, among other things. Useful pharmaceutical compositions and methods for their preparation may be found, for example, in A. R. Gennaro (ed.), Remington: The Science and Practice of Pharmacy (20th ed., 2000).

Compounds of Formula 1 may be administered orally. Oral administration may involve swallowing in which case the compound enters the bloodstream via the gastrointestinal tract. Alternatively, or additionally, oral administration may involve mucosal administration (e.g., buccal, sublingual, supralingual administration) such that the compound enters the bloodstream through the oral mucosa.

Formulations suitable for oral administration include solid, semi-solid and liquid systems such as tablets; soft or hard capsules containing multi- or nano-particulates, liquids, or powders; lozenges which may be liquid-filled; chews; gels; fast dispersing dosage forms; films; ovules; sprays; and buccal or mucoadhesive patches. Liquid formulations include suspensions, solutions, syrups and elixirs. Such formulations may be employed as fillers in soft or hard capsules (made, e.g., from gelatin or hydroxypropylmethylcellulose) and typically comprise a carrier (e.g., water, ethanol, polyethylene glycol, propylene glycol, methylcellulose, or a suitable oil) and one or more emulsifying agents, suspending agents or both. Liquid formulations may also be prepared by the reconstitution of a solid (e.g., from a sachet).

Compounds of Formula 1 may also be used in fast-dissolving, fast-disintegrating dosage forms such as those described in Liang and Chen, Expert Opinion in Therapeutic Patents (2001) 11(6):981-986.

For tablet dosage forms, depending on dose, the active pharmaceutical ingredient (API) may comprise from about 1 wt % to about 80 wt % of the dosage form or more typically from about 5 wt % to about 60 wt % of the dosage form. In addition to the API, tablets may include one or more disintegrants, binders, diluents, surfactants, glidants, lubricants, anti-oxidants, colorants, flavoring agents, preservatives, and taste-masking agents. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, C₁₋₆ alkyl-substituted hydroxypropylcellulose, starch, pregelatinized starch, and sodium alginate. Generally, the disintegrant will comprise from about 1 wt % to about 25 wt % or from about 5 wt % to about 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropylcellulose and hydroxypropylmethylcellulose. Tablets may also contain diluents, such as lactose (monohydrate, spray-dried monohydrate, anhydrous), mannitol, xylitol, dextrose, sucrose, sorbitol, microcrystalline cellulose, starch and dibasic calcium phosphate dihydrate.

Tablets may also include surface active agents, such as sodium lauryl sulfate and polysorbate 80, and glidants such as silicon dioxide and talc. When present, surface active agents may comprise from about 0.2 wt % to about 5 wt % of the tablet, and glidants may comprise from about 0.2 wt % to about 1 wt % of the tablet.

Tablets may also contain lubricants such as magnesium stearate, calcium stearate, zinc stearate, sodium stearyl fumarate, and mixtures of magnesium stearate with sodium lauryl sulfate. Lubricants may comprise from about 0.25 wt % to about 10 wt % or from about 0.5 wt % to about 3 wt % of the tablet.

Tablet blends may be compressed directly or by roller compaction to form tablets. Tablet blends or portions of blends may alternatively be wet-, dry-, or melt-granulated, melt congealed, or extruded before tableting. If desired, prior to blending one or more of the components may be sized by screening or milling or both. The final dosage form may comprise one or more layers and may be coated, uncoated, or encapsulated. Exemplary tablets may contain up to about 80 wt % of API, from about 10 wt % to about 90 wt % of binder, from about 0 wt % to about 85 wt % of diluent, from about 2 wt % to about 10 wt % of disintegrant, and from about 0.25 wt % to about 10 wt % of lubricant. For a discussion of blending, granulation, milling, screening, tableting, coating, as well as a description of alternative techniques for preparing drug products, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997).

Consumable oral films for human or veterinary use are pliable water-soluble or water-swellable thin film dosage forms which may be rapidly dissolving or mucoadhesive. In addition to the API, a typical film includes one or more film-forming polymers, binders, solvents, humectants, plasticizers, stabilizers or emulsifiers, viscosity-modifying agents, and solvents. Other film ingredients may include anti-oxidants, colorants, flavorants and flavor enhancers, preservatives, salivary stimulating agents, cooling agents, co-solvents (including oils), emollients, bulking agents, anti-foaming agents, surfactants, and taste-masking agents. Some components of the formulation may perform more than one function.

In addition to dosing requirements, the amount of API in the film may depend on its solubility. If water soluble, the API would typically comprise from about 1 wt % to about 80 wt % of the non-solvent components (solutes) in the film or from about 20 wt % to about 50 wt % of the solutes in the film. A less soluble API may comprise a greater proportion of the composition, typically up to about 88 wt % of the non-solvent components in the film.

The film-forming polymer may be selected from natural polysaccharides, proteins, or synthetic hydrocolloids and typically comprises from about 0.01 wt % to about 99 wt % or from about 30 wt % to about 80 wt % of the film.

Film dosage forms are typically prepared by evaporative drying of thin aqueous films coated onto a peelable backing support or paper, which may be carried out in a drying oven or tunnel (e.g., in a combined coating-drying apparatus), in lyophilization equipment, or in a vacuum oven.

Useful solid formulations for oral administration may include immediate release formulations and modified release formulations. Modified release formulations include delayed-, sustained-, pulsed-, controlled-, targeted-, and programmed-release. For a general description of suitable modified release formulations, see U.S. Pat. No. 6,106,864. For details of other useful release technologies, such as high energy dispersions and osmotic and coated particles, see Verma et al, Pharmaceutical Technology On-line (2001) 25(2):1-14.

Compounds of Formula 1 may also be administered directly into the blood stream, muscle, or an internal organ of the subject. Suitable techniques for parenteral administration include intravenous, intraarterial, intraperitoneal, intrathecal, intraventricular, intraurethral, intrasternal, intracranial, intramuscular, intrasynovial, and subcutaneous administration. Suitable devices for parenteral administration include needle injectors, including microneedle injectors, needle-free injectors, and infusion devices.

Parenteral formulations are typically aqueous solutions which may contain excipients such as salts, carbohydrates and buffering agents (e.g., pH of from about 3 to about 9). For some applications, however, compounds of Formula 1 may be more suitably formulated as a sterile non-aqueous solution or as a dried form to be used in conjunction with a suitable vehicle such as sterile, pyrogen-free water. The preparation of parenteral formulations under sterile conditions (e.g., by lyophilization) may be readily accomplished using standard pharmaceutical techniques.

The solubility of compounds which are used in the preparation of parenteral solutions may be increased through appropriate formulation techniques, such as the incorporation of solubility-enhancing agents. Formulations for parenteral administration may be formulated to be immediate or modified release. Modified release formulations include delayed, sustained, pulsed, controlled, targeted, and programmed release. Thus, compounds of Formula 1 may be formulated as a suspension, a solid, a semi-solid, or a thixotropic liquid for administration as an implanted depot providing modified release of the active compound. Examples of such formulations include drug-coated stents and semi-solids and suspensions comprising drug-loaded poly(DL-lactic-coglycolic) acid (PGLA) microspheres.

Compounds of Formula 1 may also be administered topically, intradermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers may include alcohol, water, mineral oil, liquid petrolatum, white petrolatum, glycerin, polyethylene glycol and propylene glycol. Topical formulations may also include penetration enhancers. See, e.g., Finnin and Morgan, J. Pharm. Sci. 88(10):955-958 (1999).

Other means of topical administration include delivery by electroporation, iontophoresis, phonophoresis, sonophoresis and microneedle or needle-free (e.g. Powderject™ and Bioject™) injection. Formulations for topical administration may be formulated to be immediate or modified release as described above.

Compounds of Formula 1 may also be administered intranasally or by inhalation, typically in the form of a dry powder, an aerosol spray, or nasal drops. An inhaler may be used to administer the dry powder, which comprises the API alone, a powder blend of the API and a diluent, such as lactose, or a mixed component particle that includes the API and a phospholipid, such as phosphatidylcholine. For intranasal use, the powder may include a bioadhesive agent, e.g., chitosan or cyclodextrin. A pressurized container, pump, sprayer, atomizer, or nebulizer, may be used to generate the aerosol spray from a solution or suspension comprising the API, one or more agents for dispersing, solubilizing, or extending the release of the API (e.g., EtOH with or without water), one or more solvents (e.g., 1,1,1,2-tetrafluoroethane or 1,1,1,2,3,3,3-heptafluoropropane) which serve as a propellant, and an optional surfactant, such as sorbitan trioleate, oleic acid, or an oligolactic acid. An atomizer using electrohydrodynamics may be used to produce a fine mist.

Prior to use in a dry powder or suspension formulation, the drug product is usually comminuted to a particle size suitable for delivery by inhalation (typically 90% of the particles, based on volume, having a largest dimension less than 5 microns). This may be achieved by any appropriate size reduction method, such as spiral jet milling, fluid bed jet milling, supercritical fluid processing, high pressure homogenization, or spray drying.

Capsules, blisters and cartridges (made, for example, from gelatin or hydroxypropylmethyl cellulose) for use in an inhaler or insufflator may be formulated to contain a powder mixture of the active compound, a suitable powder base such as lactose or starch, and a performance modifier such as L-leucine, mannitol, or magnesium stearate. The lactose may be anhydrous or monohydrated. Other suitable excipients include dextran, glucose, maltose, sorbitol, xylitol, fructose, sucrose, and trehalose.

A suitable solution formulation for use in an atomizer using electrohydrodynamics to produce a fine mist may contain from about 1 μg to about 20 mg of the API per actuation and the actuation volume may vary from about 1 μL to about 100 μL. A typical formulation may comprise one or more compounds of Formula 1, propylene glycol, sterile water, EtOH, and NaCl. Alternative solvents, which may be used instead of propylene glycol, include glycerol and polyethylene glycol.

Formulations for inhaled administration, intranasal administration, or both, may be formulated to be immediate or modified release using, for example, PGLA. Suitable flavors, such as menthol and levomenthol, or sweeteners, such as saccharin or sodium saccharin, may be added to formulations intended for inhaled/intranasal administration.

In the case of dry powder inhalers and aerosols, the dosage unit is determined by means of a valve that delivers a metered amount. Units are typically arranged to administer a metered dose or “puff” containing from about 10 μg to about 1000 μg of the API. The overall daily dose will typically range from about 100 μg to about 10 mg which may be administered in a single dose or, more usually, as divided doses throughout the day.

The active compounds may be administered rectally or vaginally, e.g., in the form of a suppository, pessary, or enema. Cocoa butter is a traditional suppository base, but various alternatives may be used as appropriate. Formulations for rectal or vaginal administration may be formulated to be immediate or modified release as described above.

Compounds of Formula 1 may also be administered directly to the eye or ear, typically in the form of drops of a micronized suspension or solution in isotonic, pH-adjusted, sterile saline. Other formulations suitable for ocular and aural administration include ointments, gels, biodegradable implants (e.g. absorbable gel sponges, collagen), non-biodegradable implants (e.g. silicone), wafers, lenses, and particulate or vesicular systems, such as niosomes or liposomes. The formulation may include one or more polymers and a preservative, such as benzalkonium chloride. Typical polymers include crossed-linked polyacrylic acid, polyvinylalcohol, hyaluronic acid, cellulosic polymers (e.g., hydroxypropylmethylcellulose, hydroxyethylcellulose, methyl cellulose), and heteropolysaccharide polymers (e.g., gelan gum). Such formulations may also be delivered by iontophoresis. Formulations for ocular or aural administration may be formulated to be immediate or modified release as described above.

To improve their solubility, dissolution rate, taste-masking, bioavailability, or stability, compounds of Formula 1 may be combined with soluble macromolecular entities, including cyclodextrin and its derivatives and polyethylene glycol-containing polymers. For example, API-cyclodextrin complexes are generally useful for most dosage forms and routes of administration. Both inclusion and non-inclusion complexes may be used. As an alternative to direct complexation with the API, the cyclodextrin may be used as an auxiliary additive, i.e. as a carrier, diluent, or solubilizer. Alpha-, beta- and gamma-cyclodextrins are commonly used for these purposes. See, e.g., WO 91/11172, WO 94/02518, and WO 98/55148.

As noted above, one or more compounds of Formula 1, including compounds specifically named above, and their pharmaceutically active complexes, salts, solvates and hydrates, may be combined with each other or with one or more other active pharmaceutically active compounds to treat various diseases, conditions and disorders. In such cases, the active compounds may be combined in a single dosage form as described above or may be provided in the form of a kit which is suitable for coadministration of the compositions. The kit comprises (1) two or more different pharmaceutical compositions, at least one of which contains a compound of Formula 1; and (2) a device for separately retaining the two pharmaceutical compositions, such as a divided bottle or a divided foil packet. An example of such a kit is the familiar blister pack used for the packaging of tablets or capsules. The kit is suitable for administering different types of dosage forms (e.g., oral and parenteral) or for administering different pharmaceutical compositions at separate dosing intervals, or for titrating the different pharmaceutical compositions against one another. To assist with patient compliance, the kit typically comprises directions for administration and may be provided with a memory aid.

For administration to human patients, the total daily dose of the claimed and disclosed compounds is typically in the range of about 0.1 mg to about 3000 mg depending on the route of administration. For example, oral administration may require a total daily dose of from about 1 mg to about 3000 mg, while an intravenous dose may only require a total daily dose of from about 0.1 mg to about 300 mg. The total daily dose may be administered in single or divided doses and, at the physician's discretion, may fall outside of the typical ranges given above. Although these dosages are based on an average human subject having a mass of about 60 kg to about 70 kg, the physician will be able to determine the appropriate dose for a patient (e.g., an infant) whose mass falls outside of this weight range.

As noted above, the compounds of Formula 1 may be used to treat diseases, disorders, and conditions for which activation of SSTR4 is indicated. Such diseases, disorders, and conditions generally relate to any unhealthy or abnormal state in a subject for which the activation of SSTR4 provides a therapeutic benefit. More particularly, the compounds of Formula 1 may be used to treat CNS diseases, disorders or conditions, including Alzheimer's disease, and other forms of dementia (i.e., major or mild neurocognitive disorders) associated with one or more medical conditions, including frontotemporal lobar degeneration, Lewy body disease, vascular disease, traumatic brain injury, substance or medication use, HIV infection, prion disease, Parkinson's disease, and Huntington's disease. The compounds of Formula 1 may also be used to treat major or mild neurocognitive disorders associated with depression, schizophrenia, bipolar disorder, and autism. In addition, the compounds of Formula 1 may be used to treat anxiety and to treat epilepsy. The compounds of Formula 1 may be also used to treat pain.

The claimed and disclosed compounds may be combined with one or more other pharmacologically active compounds or therapies to treat one or more disorders, diseases or conditions for which SSTR4 is indicated. Such combinations may offer significant therapeutic advantages, including fewer side effects, improved ability to treat underserved patient populations, or synergistic activity. For example, compounds of Formula 1, which include compounds specifically named above, and their pharmaceutically acceptable complexes, salts, solvates and hydrates, may be administered simultaneously, sequentially or separately in combination with one or more compounds or therapies for treating Alzheimer's disease, including beta-secretase inhibitors, gamma-secretase inhibitors, HMG-CoA reductase inhibitors, nonsteroidal anti-inflammatory drugs (NSAIDs, such as apazone, aspirin, celecoxib, diclofenac (with and without misoprostol), diflunisal, etodolac, fenoprofen, flurbiprofen, ibuprofen, indomethacin, ketoprofen, meclofenamate sodium, mefenamic acid, meloxicam, nabumetone, naproxen, oxaprozin, phenylbutazone, piroxicam, choline and magnesium salicylates, salsalate, and sulindac), vitamin E, and anti-amyloid antibodies. Specific examples of compounds used to treat Alzheimer's disease include donepezil, rivastigmine, memantine, and galantamine.

In addition to drugs used to improve cognition, the compounds of Formula 1 may be combined with sedatives, hypnotics, anxiolytics, antipsychotics, tranquilizers, and other medications that are used in the treatment of Alzheimer's disease. For example, the compounds of Formula 1 may be combined with one or more agents for treating depression (antidepressants) and/or schizophrenia (atypical or typical antipsychotics) including amitriptyline, amoxapine, aripiprazole, asenapine, bupropion, chlordiazepoxide, citalopram, chlorpromazine, clozapine, desipramine, desvenlafaxine, doxepin, duloxetine, escitalopram, fluoxetine, fluoxetine, fluphenazine, haloperidol, iloperidone, imipramine, isocarboxazid, lamotrigine, levomilnacipran, lurasidone, mirtazapine, nefazodone, nortriptyline, olanzapine, paliperidone, paroxetine, perphenazine, phenelzine, protriptyline, quetiapine, risperidone, selegiline, sertraline, tranylcypromine, trazodone, trimipramine, venlafaxine, vilazodone, and vortioxetine, and ziprasidone.

Likewise, the compounds of Formula 1 may be combined with one or more agents for treating anxiety (anxiolytics) including benzodiazepines (alprazolam, chlordiazepoxide, clobazepam, clonazepam, clorazepate, diazepam, estazolam, flurazepam, lorazepam, midazolam, oxazepam, prazepam, quazepam, temazepam, and triazolam), antihistamines (hydroxyzine), non-benzodiazepines (eszopiclone, zaleplon, zolpidem, and zopiclone) and buspirone.

The compounds of Formula 1 may also be combined with one or more agents for treating epilepsy (antiepileptics or anticonvulsants) including acetazolamide, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, vigabatrin, and zonisamide.

### Biological Activity

The biological activity of the compound of Formula 1 with respect to SSTR4 may be determined using the following in vitro and in vivo methods.

Inhibition of Forskolin Stimulated cAMP in Cells Overexpressing SSTR4

This cell-based assay measures the ability of compounds to inhibit forskolin stimulated cAMP in CHO-K1 cells overexpressing SSTR4. CHO-K1 cells overexpressing SSTR4 (CHO-SSTR4) are purchased from DiscoveRx (product code 95-0059C2). The CHO-SSTR4 cells are maintained in F12K media with 10% Fetal Bovine Serum (Hyclone), 1% Pen/Strep (Life Technologies), and 800 μg/mL G418 (Life Technologies). To perform the assay, 3000 cells are plated per well in white 384-well plate (Corning 3570) in 50 μL complete media and the cells are allowed to attach for 16 hours in a 37° C., 5% CO₂ incubator. The next day, the culture media is removed from the cells and the cells are washed (added then removed) with Krebs Ringer Buffer (ZenBio, KRB-1000 mL). Test compounds are suspended in DMSO and diluted in stimulation buffer: Krebs Ringer Buffer plus 0.5% BSA (Roche), 300 μM IBMX (Sigma), and 350 nM forskolin (Sigma). The cells are incubated in 10 μL compound/stimulation buffer for 30 minutes at room temperature. Cellular cAMP levels are detected with a HTRF LANCE Ultra cAMP kit (Perkin Elmer, catalog number TRF0264).

The assay is performed in accordance with the manufacturer's instructions. Five L of diluted Eu—W8044 labeled streptavidin (dilution: 1:50 in cAMP Detection Buffer) is added to each well. Then 5 μL of diluted biotin cAMP (dilution: 1:150 in cAMP Detection Buffer) is added to each well. The plates are covered and allowed to incubate for 60 minutes at room temperature on a shaker. HTRF (665 nm/615 nm) is read on a Perkin Elmer ENVISION plate reader. The pEC₅₀ values are generated using Activity Base for Screening Data Management.

### SSTR4 I-125 Somatostatin Competition Binding Assay

This membrane-based assay measures the ability of compounds to competitively inhibit binding of I-125 labeled somatostatin to SSTR4 in membranes from CHO-K1 that overexpress SSTR4. Membranes from CHO-K1 cells overexpressing SSTR4 are purchased from Perkin Elmer (catalog number ES-524-M400UA). Test compounds are suspended in DMSO and then diluted in assay buffer (25 mM HEPES pH 7.4, 10 mM MgCl₂, 1 mM CaCl₂, 0.5% BSA) plus 0.2 nM I-125 labeled somatostatin (Perkin Elmer catalog number NEX389). Fifty L of compound/I-125 somatostatin in assay buffer are added per well to 96-well poly-propylene plate. Then 1 μg of SSTR4 membranes in 50 μL assay buffer are added per well. The Plate is incubated for 60 minutes at room temperature. FilterMat A filters (Perkin Elmer catalog number 1450-421) are pre-soaked in 0.5% PEI (Sigma catalog number P3143). The contents of the assay plate are transferred to filters with a TomTech harvester and washed 5 times with 20 mM HEPES, 100 mM NaCl. The filters are dried in a microwave oven then transferred to sample bag containing a scintillator sheet (Perkin Elmer catalog number 1450-441). The scintillator sheets are melted to filters using a heat block. The filters are then read in a MicroBeta scintillation counter. Binding Ki curves are generated using Activity Base for Screening Data Management and the results are reported as pIC₅₀.

### SSTR1 I-125 Somatostatin Competition Binding Assay for Selectivity Versus SSTR1

This membrane-based assay measures the ability of compounds to competitively inhibit binding of I-125 labeled somatostatin to SSTR1 in membranes from CHO-K1 that overexpress SSTR1. Membranes from CHO-K1 cells overexpressing SSTR1 are purchased from Perkin Elmer (catalog number ES-520-M400UA). Test compounds are suspended in DMSO and then diluted in assay buffer (25 mM HEPES pH 7.4, 10 mM MgCl₂, 1 mM CaCl₂, 0.5% BSA) plus 0.4 nM I-125 labeled somatostatin (Perkin Elmer catalog number NEX389). Fifty μL of compound/I-125 somatostatin in assay buffer are added per well to 96-well poly-propylene plate. Then 10 μg of SSTR1 membranes in 50 μL assay buffer are added per well. The plate is incubated for 60 minutes at room temperature. FilterMat A filters (Perkin Elmer catalog number 1450-421) are pre-soaked in 0.5% PEI (Sigma catalog number P3143). The contents of the assay plate are transferred to filters with a TomTech harvester and washed 5 times with 20 mM HEPES, 100 mM NaCl. The filters are dried in a microwave oven then transferred to sample bag containing a scintillator sheet (Perkin Elmer catalog number 1450-441). The scintillator sheets are melted to the filters using a heat block. The filters are then read in a MicroBeta scintillation counter. Binding Ki curves are generated using Activity Base for Screening Data Management and the results are reported as pIC₅₀.

The following in vitro assay may be used to assess the ability of a compound of Formula 1 to enter the CNS through the blood-brain barrier.

### LLC-PK1 Assay (Reported as Apparent Permeability and Efflux Ratio)

Lilly Laboratories & Company porcine kidney cells (LLC-PK1) are transfected with Multidrug resistance protein 1 (MDR1) and are maintained in accordance with the provider's instructions in Gibco Medium 199 (Fisher Scientific Cat #11150067). Medium 199 is supplemented with 10% heat-inactivated fetal bovine serum (Gibco Cat #16000-044), 0.5 mg/mL Geneticin (Gibco #10131035) and colchicine 200 nM (an inducer of P-gp, Sigma Cat #C9754). The cells are seeded onto the apical side of HTS-Transwell-96 Plates (0.4 μm pore size, Corning Cat #3381) at a density of 6.25×10³ cells per well with 75 μL and 250 μL of Medium 199 in apical and basolateral wells, respectively, and are incubated at 37° C./5% CO₂. Fresh Medium 199 media is exchanged in the apical and basolateral compartments after 72 hours and cells are allowed to grow into a monolayer for 144 hours before beginning the experimental incubation. Incubations are performed in Media 199 (Fisher Scientific Cat #11150067) at pH 7.4 with 1% bovine serum albumin (Sigma, Cat #A9418) and 10 mM HEPES (Fisher Scientific, Cat #15630080). Media 199 is removed and cells are rinsed with warm (37° C.) Media 199. Media 199 with test compound at 1 μM substrate concentration (0.1% v/v DMSO) is added to either the apical or basolateral compartment (75 μL or 250 μL, respectively) and blank Media 199 is added to compartment which lacks test compound in singlicate. The cells are incubated for 120 minutes at 37° C./5% CO₂. At the end of the incubation period, a 50 μL sample is removed from each receiver compartment and diluted into 100 μL of acetonitrile (Fisher Scientific, Cat #A996SK4)+100 ng/mL diclofenac (internal standard, Sigma #15307-79-6). The samples are centrifuged at 2000 RCF, for 10 minutes at 4° C., after which 75 μL of supernatant is transferred to a new microplate and diluted with 75 μL of HPLC grade water (Fisher Scientific, Cat #W64). Samples are analyzed using Multiple Reaction Monitoring on an ABSciex triple quadrupole mass spectrometer coupled to high performance liquid chromatography pump instrumentation optimized for the detection of test articles through a Kinetix 2.1×50 mm C18 100 Å column (Phenomenex, Cat #00B-4605-AN).

Apparent permeability (Pₐₚₚ) values and efflux ratio are calculated using the following equations:

$${P}_{app A - B} ( nm \times {sec}^{- 1} ) = \frac{{Conc}_{BL} \times 0.25 \times 10000}{A \times t \times 10} ;$$

$${P}_{app B - A} ( nm \times {sec}^{- 1} ) = \frac{{Conc}_{AP} \times 0.075 \times 10000}{A \times t \times 10} ;$$

$$ER = \frac{{P}_{app B - A}}{{P}_{app A - B}} ;$$

where P_{app A-B} is the apparent permeability from the apical well to basolateral well; P_{app B-A} is the apparent permeability from the basolateral well to the apical well; Conc_{BL} is the basolateral well concentration; Conc_{AP} is the apical well concentration; A is the well surface area (cm²), which for the above assay is 0.143 cm²; t is the incubation time (seconds), which for the above assay is 7200 seconds; and ER is the P-gp mediated efflux ratio.

### EXAMPLES

The following examples are intended to be illustrative and non-limiting and represent specific embodiments of the present invention.

¹H Nuclear magnetic resonance (NMR) spectra were obtained for many of the compounds in the following examples. Characteristic chemical shifts (δ) are given in parts-per-million downfield from tetramethylsilane using conventional abbreviations for designation of major peaks, including s (singlet), d (doublet), t (triplet), q (quartet), m (multiplet), and br (broad). The following abbreviations are used for common solvents: CDCl₃ (deuterochloroform), DMSO-d₆ (deuterodimethylsulfoxide), CD₃OD (deuteromethanol), CD₃CN (deuteroacetonitrile), and THF-d₈ (deuterotetrahydrofuran). The mass spectra (m/z for [M+H]⁺) were recorded using either electrospray ionization (ESI-MS) or atmospheric pressure chemical ionization (APCI-MS) mass spectrometry.

Where indicated, products of certain preparations and examples are purified by mass-triggered HPLC, flash chromatography, preparative TLC or SFC. Reverse phase chromatography is typically carried out on a column (e.g., Phenomenex Gemini® NX—C18, 5 μm, ID 30 mm×100 mm) under acidic conditions (“Acid Mode”) eluting with ACN and water mobile phases containing 0.035% and 0.05% trifluoroacetic acid (TFA), respectively, or under basic conditions (“Basic Mode”) eluting with water and 20/80 (v/v) water/acetonitrile mobile phases, both containing 10 mM NH₄HCO₃ (pH 9.5-10). Preparative TLC is typically carried out on silica gel 60 F₂₅₄ plates. The preparations and examples may employ SFC to separate enantiomers. After isolation by chromatography, the solvent is removed and the product is obtained by drying in a centrifugal evaporator (e.g., GeneVac™) rotary evaporator, evacuated flask, etc. Reactions in an inert (e.g., nitrogen) or reactive (e.g., H₂) atmosphere are typically carried out at a pressure of about 1 atmosphere (14.7 psi).

### Preparation 1: 1-(2-cyclopropylphenoxy)cyclopropane-1-carboxylic acid

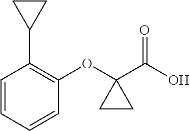 [SMILES: O=C(O)C1(Oc2ccccc2C2CC2)CC1]

### Step A: methyl 4-bromo-2-(2-bromophenoxy)butanoate

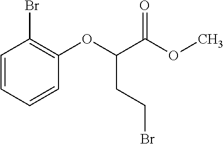 [SMILES: COC(=O)C(CCBr)Oc1ccccc1Br]

A mixture of 2-bromophenol (5.00 g, 28.9 mmol), methyl 2,4-dibromobutanoate (9.01 g, 34.7 mmol) and K₂CO₃ (7.99 g, 57.8 mmol) in DMF (100 mL) was degassed and purged with nitrogen (3×) and then stirred at 20° C. for 12 hours under nitrogen atmosphere. The reaction mixture was diluted with water (200 mL) and extracted with EtOAc (2×100 mL). The combined organic layers were dried over Na₂SO₄ and concentrated under reduced pressure. The crude residue was purified by silica column chromatography using a gradient of petroleum ether/EtOAc (1:0 to 10:1) to give the title compound as a colorless oil (8.3 g, 82%). ¹H NMR (400 MHz, CDCl₃) δ ppm 2.43-2.52 (m, 1H), 2.56-2.65 (m, 1H), 3.62-3.70 (m, 1H), 3.70-3.76 (m, 1H), 3.78 (s, 3H), 4.89 (dd, J=9.0, 3.7 Hz, 1H), 6.79 (dd, J=8.3, 1.2 Hz, 1H), 6.89 (td, J=7.6, 1.3 Hz, 1H), 7.21-7.26 (m, 1H), 7.24 (dd, J=7.8, 1.2 Hz, 1H), 7.22-7.26 (m, 1H), 7.22-7.26 (m, 1H), 7.55 (dd, J=8.0, 1.6 Hz, 1H).

### Step B: methyl 1-(2-bromophenoxy)cyclopropane-1-carboxylate

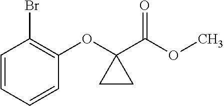

To a solution of methyl 4-bromo-2-(2-bromophenoxy)butanoate (8.3 g, 23.6 mmol) in THF (120 mL) was added LiHMDS (1 M in THF, 52 mL, 52 mmol) at −78° C. The reaction mixture was stirred at −78° C. for 2 hours under nitrogen and then poured into aqueous NH₄Cl (200 mL) and extracted with EtOAc (3×200 mL). The combined organic layers were dried over Na₂SO₄ and concentrated under reduced pressure. The crude residue was purified by silica column chromatography using a gradient of petroleum ether/EtOAc (1:0 to 10:1) to give the title compound as a colorless oil (5.05 g, 79%). ¹H NMR (400 MHz, CDCl₃) δ ppm 1.36-1.41 (m, 2H), 1.63-1.69 (m, 2H), 3.73 (s, 3H), 6.84-6.90 (m, 1H), 6.95 (dd, J=8.3, 1.0 Hz, 1H), 7.20-7.25 (m, 1H), 7.53 (dd, J=7.9, 1.4 Hz, 1H).

### Step C: methyl 1-(2-cyclopropylphenoxy)cyclopropane-1-carboxylate

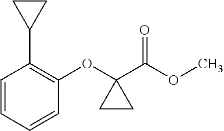 [SMILES: COC(=O)C1(Oc2ccccc2C2CC2)CC1]

A mixture of methyl 1-(2-bromophenoxy)cyclopropane-1-carboxylate (0.500 g, 1.84 mmol), cyclopropylboronic acid (238 mg, 2.77 mmol), Pd(OAc)₂ (41.4 mg, 184 μmol), PCy₃ (51.7 mg, 184 μmol) and K₃PO₄ (1.37 g, 6.46 mmol) in toluene (10 mL) and water (0.5 mL) was degassed and purged with nitrogen (3×) and then stirred at 100° C. for 12 hours under nitrogen atmosphere. The reaction mixture was poured into aqueous NH₄Cl (20 mL) and extracted with EtOAc (2×20 mL). The combined organic layers were dried over Na₂SO₄ and concentrated under reduced pressure. The crude residue was purified by silica column chromatography using a gradient of petroleum ether/EtOAc (1:0 to 20:1) to give the title compound as a light yellow oil (400 mg, 84% yield, 90% purity). ¹H NMR (400 MHz, CDCl₃) δ ppm 0.59-0.66 (m, 2H), 0.87-0.94 (m, 2H), 1.30-1.37 (m, 2H), 1.60-1.67 (m, 2H), 2.07-2.18 (m, 1H), 3.74 (s, 3H), 6.81-6.95 (m, 3H), 7.03-7.11 (m, 1H); ESI-MS m/z [M+H]⁺ 233.2.

### Step D: 1-(2-cyclopropylphenoxy)cyclopropane-1-carboxylic acid

To a solution of methyl 1-(2-cyclopropylphenoxy)cyclopropane-1-carboxylate (400 mg, 1.55 mmol) in THE (5 mL) was added aqueous LiOH·H₂O (1.5 M, 3.15 mL, 4.72 mmol). The mixture was stirred at 20° C. for 12 hours and then diluted with water (10 mL) and extracted with EtOAc (2×10 mL). The aqueous layer was acidified with 2 M HCl to pH 4 and then extracted with EtOAc (3×10 mL). The combined organic layers were concentrated under reduced pressure to give the title compound as a light yellow solid (230.2 mg, 65% yield, 95% purity). ¹H NMR (400 MHz, CDCl₃) δ ppm 0.60-0.67 (m, 2H), 0.87-0.93 (m, 2H), 1.38-1.45 (m, 2H), 1.67-1.75 (m, 2H), 2.06-2.16 (m, 1H), 6.82-6.87 (m, 1H), 6.88-6.97 (m, 2H), 7.05-7.13 (m, 1H); ESI-MS m/z [M+H]⁺ 219.1.

### Preparation 2: 1-(((6-chloropyridin-2-yl)(methyl)amino)methyl)cyclopropane-1-carboxylic acid

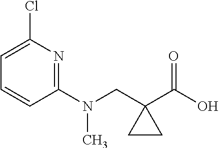 [SMILES: CN(CC1(C(=O)O)CC1)c1cccc(Cl)n1]

### Step A: methyl 1-(((6-chloropyridin-2-yl)(methyl)amino)methyl)cyclopropane-1-carboxylate

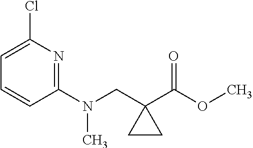 [SMILES: COC(=O)C1(CN(C)c2cccc(Cl)n2)CC1]

In a 40 mL vial, 6-chloro-N-methylpyridin-2-amine (0.100 g, 0.701 mmol) was dissolved in DMF (3 mL) to give a colorless solution. Sodium hydride (60 wt %, 0.042 g, 1.052 mmol) was added, followed by methyl 1-(bromomethyl)cyclopropane-1-carboxylate (0.162 g, 0.841 mmol). The mixture was stirred at room temperature overnight and then quenched with saturated aqueous NH₄Cl and extracted with EtOAc. The organic extracts were washed with brine, dried over MgSO₄ and concentrated in vacuo to give the title compound as a brown syrup (0.179 g). ESI-MS m/z [M+H]⁺ 255.1.

### Step B: 1-(((6-chloropyridin-2-yl)(methyl)amino)methyl)cyclopropane-1-carboxylic acid

In a 40 ml vial, crude methyl 1-(((6-chloropyridin-2-yl)(methyl)amino)methyl)cyclopropane-1-carboxylate (0.179 g, 0.701 mmol) was combined with aqueous lithium hydroxide (2 M, 1.40 mL, 2.80 mmol) in dioxane (4 mL) to give a colorless solution. The mixture was stirred at room temperature overnight and then acidified with aqueous HCl to pH 5 and extracted with EtOAc. The organic extracts were dried over MgSO₄ and concentrated in vacuo to give the title compound as a light brown syrup (0.118 g, 70%). ESI-MS m/z [M+H]⁺ 241.1.

### Preparation 3: 2-(2-cyclopropylphenoxy)propanoic acid

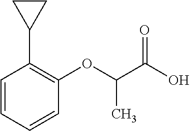

### Step A: methyl 2-(2-cyclopropylphenoxy)propanoate

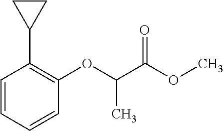 [SMILES: COC(=O)C(C)Oc1ccccc1C1CC1]

In a 100 mL round-bottomed flask, 2-cyclopropylphenol (0.200 g, 1.491 mmol) and potassium carbonate (0.618 g, 4.47 mmol) were combined in DMF (8 mL) to give a white suspension. Methyl 2-chloropropanoate (0.219 g, 1.789 mmol) was added and the reaction mixture was stirred at room temperature overnight. The mixture was diluted with water and extracted with EtOAc. The organic phase was dried over MgSO₄ and concentrated in vacuo. The residue was purified by automated flash silica column chromatography (ISCO, 40 g RediSep Rf Gold® column) using a gradient of 0-20% EtOAc in heptanes. The product-containing fractions were combined and concentrated to give the title compound as a colorless oil (307 mg, 94%).

### Step B: 2-(2-cyclopropylphenoxy)propanoic acid

In a 125 mL pear-shaped flask, methyl 2-(2-cyclopropylphenoxy)propanoate (0.307 g, 1.394 mmol) and aqueous lithium hydroxide (2 M, 2.79 mL, 5.58 mmol) were combined in dioxane (8 mL) to give a colorless solution. The reaction mixture was stirred at room temperature overnight and then acidified with aqueous HCl and extracted with EtOAc. The organic extracts were dried over MgSO₄ and concentrated in vacuo to give the title compound as a white solid (218 mg, 76%). ESI-MS m/z [M−CO₂H]⁺ 161.2.

### Preparation 4: 1-(2-chlorophenoxy)cyclopropane-1-carboxylic acid

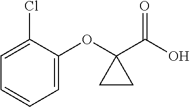 [SMILES: O=C(O)C1(Oc2ccccc2Cl)CC1]

In a 250 mL round-bottomed flask, methyl 1-(2-chlorophenoxy)cyclopropane-1-carboxylate (1.00 g, 4.41 mmol) was dissolved in dioxane (16 mL) to give a colorless solution. Aqueous lithium hydroxide (2 M, 8.82 mL, 17.65 mmol) was added. The mixture was stirred at room temperature for 4 hours and then acidified with aqueous HCl and extracted with EtOAc. The organic extracts were dried over MgSO₄ and concentrated in vacuo to give the title compound as a white solid (0.66 g, 70%). ESI-MS m/z [M−CO₂H]⁺166.9.

### Preparation 5: tert-butyl (3S,4S)-4-amino-3-methylpiperidine-1-carboxylate

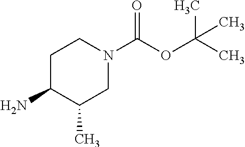

### Step A: tert-butyl (3S,4S)-4-amino-3-methylpiperidine-1-carboxylate (2S,3S)-2,3-bis(benzoyloxy)succinic acid

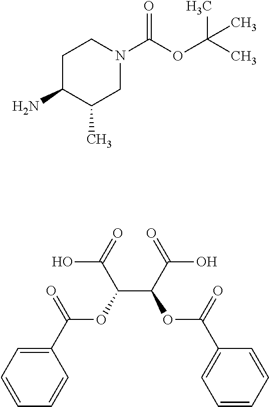 [SMILES: C[C@H]1CN(C(=O)OC(C)(C)C)CC[C@@H]1N ; O=C(O[C@H](C(=O)O)[C@H](OC(=O)c1ccccc1)C(=O)O)c1ccccc1]

To a solution of tert-butyl trans-4-amino-3-methylpiperidine-1-carboxylate (120 g, 560 mmol) in MeOH (1200 mL) at room temperature was added dibenzoyl-D-tartaric acid (201 g, 560 mmol) in MeOH (600 mL). The addition was exothermic (22° C. to 29° C.). Seed crystals (˜1 mg) were added and the mixture was stirred at room temperature overnight. The resulting slurry was filtered and washed with MeOH (360 mL, displacement wash) and then suction-dried at room temperature to give the title complex (111 g, 35%, 88.2% ee by derivatization). The material was repeatedly recrystallized from EtOH to give the title complex with increased ee (73.6 g, 98.4% ee). ¹H NMR (400 MHz, DMSO-d₆) δ ppm 0.86 (d, J=6.5 Hz, 3H), 1.20-1.57 (m, 11H), 1.86 (br dd, J=12.8, 3.3 Hz, 1H), 2.23-2.48 (m, 1H), 2.55-2.56 (m, 1H), 2.78 (br d, J=3.8 Hz, 2H), 3.66-3.95 (m, 2H), 5.65 (s, 2H), 7.40-7.56 (m, 4H), 7.58-7.69 (m, 2H), 7.95 (d, J=7.5 Hz, 4H).

### Step B: tert-butyl (3S,4S)-4-amino-3-methylpiperidine-1-carboxylate

To a 2 μL flask were charged tert-butyl (3S,4S)-4-amino-3-methylpiperidine-1-carboxylate (2S,3S)-2,3-bis(benzoyloxy)succinic acid (71.0 g, 124 mmol) and DCM (1000 mL) to furnish a white suspension. Next, aqueous Na₂CO₃ (2 M, 1000 mL) was added to the suspension over a one-minute period with stirring to provide two colorless phases. The two phases were stirred at room temperature for 5 minutes and then transferred to a 4 μL separatory funnel, shaken vigorously and separated. Additional H₂O (500 mL) was added to the aqueous phase to solubilize salts. The aqueous phase was washed with DCM (2×500 mL) and the organic extracts were combined, dried over Na₂SO₄ (430 g), filtered, rinsed with DCM, and dried on a rotovap at 30° C. The oil was dissolved in acetonitrile (150 mL), concentrated via rotary evaporation, and dried on the rotovap in vacuo at 30° C. to give the title compound as a colorless oil (26.6 g, quantitative). ¹H NMR (400 MHz, CD₃OD) δ ppm 0.97 (d, J=6.5 Hz, 3H), 1.19-1.35 (m, 2H), 1.45 (s, 9H), 1.75-1.83 (m, 1H), 2.32-2.53 (m, 2H), 2.80 (br t, J=12.0 Hz, 1H), 3.89-3.98 (m, 1H), 4.04 (ddt, J=13.5, 4.6, 2.4 Hz, 1H); ESI-MS m/z [M+Na]⁺ 237.2.

### Preparation 6: 2,2-difluoro-3-phenylpropanoic acid

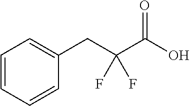 [SMILES: O=C(O)C(F)(F)Cc1ccccc1]

A solution of (bromomethyl)benzene (324 μL, 2.73 mmol), ethyl 2,2-difluoro-2-iodoacetate (568 mg, 2.27 mmol) and copper (375 mg, 5.91 mmol) in DMSO (5.98 mL) was stirred on a hot plate at 60° C. overnight. The reaction mixture was then filtered under vacuum and extracted with diethyl ether (2×). The combined extracts were concentrated in vacuo, dissolved in MeOH (4.5 mL), treated with aqueous lithium hydroxide (3 M, 2.27 mL, 6.82 mmol) and stirred at room temperature overnight. The reaction mixture was acidified to pH 4 by the dropwise addition of 1 N HCl and then extracted with EtOAc (1×) and DCM (2×). The organic layers were combined, dried over Na₂SO₄ and filtered. Solvent was removed under reduced pressure to give the crude title compound (139 mg, 33%).

### Preparation 7: 2,2-difluoro-2-phenoxyacetic acid

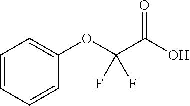 [SMILES: O=C(O)C(F)(F)Oc1ccccc1]

To a solution of phenol (1.39 g, 14.8 mmol) in dioxane (24.6 mL) was added sodium hydride (60 wt %, 0.59 g, 14.8 mmol) portion wise at room temperature. The solution was vigorously stirred for 30 minutes. Next, sodium 2-chloro-2,2-difluoroacetate (1.50 g, 9.84 mmol) was added in one portion at room temperature. The reaction mixture was stirred on a hot plate at 110° C. for 16 hours and then cooled to room temperature, quenched with water, and acidified to pH 1 by the dropwise addition of 1 N HCl. The solution was extracted with ether (1×50 mL) and EtOAc (2×75 mL). The organic layers were combined, concentrated under reduced pressure and purified by automated flash silica column chromatography (ISCO, 24 μg RediSep Rf Gold® column) using 30% EtOAc in heptanes. The title compound was obtained as an orange-brown liquid (1.25 g, 68%). ¹H NMR (400 MHz, CDCl₃) δ ppm 7.24-7.29 (m, 2H), 7.31 (dt, J=7.4, 1.6 Hz, 1H), 7.38-7.44 (m, 2H), 9.48 (br s, 1H); ¹⁹F NMR (376 MHz, CDCl₃) S ppm −76.55 (s, 2 F).

### Preparation 8: 2-(2-cyanophenoxy)-2,2-difluoroacetic acid

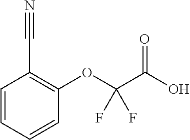 [SMILES: N#Cc1ccccc1OC(F)(F)C(=O)O]

### Step A: ethyl 2-(2-cyanophenoxy)-2,2-difluoroacetate

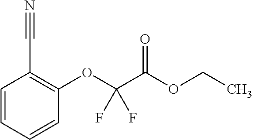 [SMILES: CCOC(=O)C(F)(F)Oc1ccccc1C#N]

To a suspension of sodium hydride (60 wt %, 0.739 g, 18.5 mmol) in DMA (42.0 mL) was added 2-hydroxybenzonitrile (2.00 g, 16.8 mmol) portion wise at room temperature. The reaction mixture was stirred for 10 minutes. Ethyl 2-bromo-2,2-difluoroacetate (2.69 mL, 21.0 mmol) was added dropwise via syringe at room temperature. The brown reaction mixture was stirred on a hot plate at 100° C. for 48 hours and then allowed to cool to room temperature. Diethyl ether (150 mL) was added. The organic layer was washed with water (75 mL) and brine (75 mL), dried over Na₂SO₄ and filtered under vacuum. The solvent was removed under reduced pressure and the crude product (an orange brown oil) was purified by automated flash silica column chromatography (ISCO, 40 μg RediSep Rf Gold® column, dry loading) using 15% EtOAc in heptanes, followed by a gradient of 30-100% EtOAc in heptanes. The product-containing fractions were combined, concentrated under reduced pressure and further purified by preparative HPLC (Phenomenex Gemini® NX—C18, 5 μm, ID 30 mm×100 mm) using a gradient of 10-100% ACN in water (Acid Mode). The title compound was obtained as an orange oil (307 mg, 7.6%). ¹H NMR (400 MHz, CDCl₃) δ ppm 1.41 (t, J=7.2 Hz, 3H), 4.44 (q, J=7.2 Hz, 2H), 7.38 (td, J=7.6, 1.0 Hz, 1H), 7.43-7.47 (m, 1H), 7.59-7.67 (m, 1H), 7.71 (dd, J=7.8, 1.8 Hz, 1H); ¹⁹F NMR (376 MHz, CDCl₃) δ ppm −76.61 (s, 2 F).

### Step B: 2-(2-cyanophenoxy)-2,2-difluoroacetic acid

A solution of ethyl 2-(2-cyanophenoxy)-2,2-difluoroacetate (307 mg, 1.28 mmol) in THE (4.26 mL) was treated with aqueous LiOH (3 M, 1.28 mL, 3.83 mmol) at room temperature. The resulting light-yellow mixture was stirred at room temperature overnight and then acidified to pH 1 by the dropwise addition of 1 N HCl (10 mL). The solution was transferred to a separatory funnel and extracted with diethyl ether (1×50 mL) and EtOAc (2×50 mL). The organic layers were combined, dried over MgSO₄, filtered and concentrated under reduced pressure. The product was purified by automated flash silica column chromatography (ISCO, 12 μg RediSep Rf Gold® column, dry loading) using a gradient of 0-100% EtOAc in heptanes to give the title compound as an orange-brown oil (203 mg, 75%). ¹H NMR (400 MHz, CDCl₃) δ ppm 7.29 (br s, 1H), 7.37-7.42 (m, 1H), 7.43-7.51 (m, 1H), 7.62-7.69 (m, 1H), 7.72 (ddd, J=7.8, 3.0, 1.5 Hz, 1H).

### Preparation 9: 2-(2-chlorophenoxy)-2,2-difluoroacetic acid

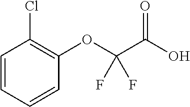 [SMILES: O=C(O)C(F)(F)Oc1ccccc1Cl]

To a solution of 2-chlorophenol (1.08 g, 8.38 mmol) in dioxane (30.5 mL) was added sodium hydride (0.457 g, 11.4 mmol) at ambient temperature. The solution was stirred for 30 minutes. Sodium 2-bromo-2,2-difluoroacetate (1.50 g, 7.62 mmol) was added in one portion at ambient temperature. Once bubbling subsided, the reaction mixture was stirred on a hot plate at 105° C. for 16 hours. The reaction mixture was cooled to ambient temperature, quenched with water (30 mL) acidified to pH 1 by dropwise addition of 6 N HCl, and extracted with EtOAc (2×50 mL). The organic layers were combined, washed with brine (20 mL), dried over Na₂SO₄ and concentrated in vacuo. The residue was purified by automated flash silica column chromatography (ISCO, 120 μg RediSep Rf Gold® column) using a gradient of 10-100% EtOAc in heptanes to give the title compound as a yellow oil (1.03 g, 61%). ¹H NMR (400 MHz, DMSO-d₆) δ ppm 7.33-7.39 (m, 1H) 7.39-7.46 (m, 2H), 7.62 (dt, J=7.6, 0.9 Hz, 1H).

### Preparation 10: 2-(3-chlorophenoxy)-2,2-difluoroacetic acid

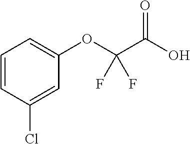 [SMILES: O=C(O)C(F)(F)Oc1cccc(Cl)c1]

To a solution of 3-chlorophenol (1.077 g, 8.38 mmol) in dioxane (30.5 mL) was added sodium hydride (0.457 g, 11.4 mmol) at ambient temperature. The solution was stirred for 30 minutes. Sodium 2-bromo-2,2-difluoroacetate (1.50 g, 7.62 mmol) was added in one portion. Once bubbling subsided, the reaction mixture was heated at 105° C. for 7 hours and then cooled to ambient temperature, quenched with water (30 mL), acidified to pH 1 by dropwise addition of 6 N HCl, and extracted with EtOAc (2×50 mL). The organic layers were combined, washed with brine (20 mL), dried over Na₂SO₄ and concentrated in vacuo. The residue was purified by automated flash silica column chromatography (ISCO, 120 g RediSep Rf Gold® column) using a gradient of 10-80% EtOAc in heptanes to give the title compound as a brown oil (0.958 g, 56%). ¹H NMR (400 MHz, DMSO-d₆) δ ppm 7.21-7.26 (m, 1H), 7.33 (t, J=2.0 Hz, 1H), 7.41 (ddd, J=8.0, 1.9, 1.0 Hz, 1H), 7.49 (t, J=8.4 Hz, 1H).

### Preparation 11: 2-(4-chlorophenoxy)-2,2-difluoroacetic acid

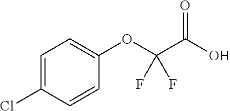 [SMILES: O=C(O)C(F)(F)Oc1ccc(Cl)cc1]

To a solution of 4-chlorophenol (1.08 g, 8.38 mmol) in dioxane (30.5 mL) was added sodium hydride (0.457 g, 11.4 mmol) at ambient temperature. The solution was stirred for 30 minutes. Sodium 2-bromo-2,2-difluoroacetate (1.50 g, 7.62 mmol) was added in one portion. The reaction mixture was stirred at 105° C. for 7 hours and then cooled to ambient temperature, quenched with water (30 mL), acidified to pH 1 by dropwise addition of 6 N HCl, and extracted with EtOAc (2×50 mL). The organic layers were combined, washed with brine (20 mL), dried over Na₂SO₄ and concentrated in vacuo. The residue was purified by automated flash silica column chromatography (ISCO, 120 μg RediSep Rf Gold® column) using a gradient of 10-80% EtOAc in heptanes to give the title compound as a yellow oil (0.665 g, 39%). ¹H NMR (400 MHz, DMSO-d₆) δ ppm 7.27 (d, J=9.0 Hz, 2H), 7.52 (d, J=9.0 Hz, 2H).

### Preparation 12: 2,2-difluoro-2-(p-tolyloxy)acetic acid

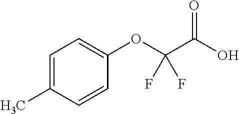

To a solution of sodium hydride (60 wt %, 0.493 g, 12.3 mmol) in dioxane (6.16 mL) was added p-cresol (0.500 g, 4.62 mmol) portion wise at room temperature. The solution was stirred for 15 minutes. Sodium 2-bromo-2,2-difluoroacetate (0.607 g, 3.08 mmol) was added portion wise at room temperature. After bubbling subsided, the reaction mixture was stirred on a hot plate at 105° C. overnight. The reaction mixture was then quenched slowly with a few drops of water, acidified to pH 1 by the dropwise addition of 1 N HCl, and extracted with EtOAc (1×) and DCM (2×). The organic layers were combined and the solvent carefully removed under reduced pressure to avoid loss of product. The title compound was purified by automated flash silica column chromatography (ISCO, 12 g RediSep Rf Gold® column, dry loading) using 30% EtOAc in heptanes (0.925 g, quantitative, 67% purity). ¹H NMR (400 MHz, DMSO-d₆) δ ppm 2.24-2.39 (m, 3H), 5.68-5.81 (m, 1H), 7.03-7.18 (m, 2H), 7.18-7.32 (m, 2H).

### Preparation 13: 2,2-difluoro-2-(4-fluorophenoxy)acetic acid

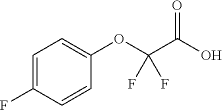 [SMILES: O=C(O)C(F)(F)Oc1ccc(F)cc1]

To a solution of sodium hydride (60 wt %, 203 mg, 5.08 mmol) in dioxane (5 mL) was added 4-fluorophenol (427 mg, 3.81 mmol) portion wise at room temperature. The solution was stirred at RT for 15 minutes. Next, sodium 2-bromo-2,2-difluoroacetate (500 mg, 2.54 mmol) was added portion wise at room temperature. After bubbling ceased, the mixture was heated at 100° C. overnight. The reaction was then quenched with the addition of water (20 mL) and the mixture was extracted with EtOAc. The aqueous layer was adjusted to pH 2 by the addition of 1 M HCl and extracted with EtOAc. The organic phase was dried over MgSO₄ and concentrated under reduced pressure. The residue was purified by automated flash silica column chromatography (ISCO®) using a gradient of 0-100% EtOAc in heptanes to give the title compound as an orange oil (262 mg, 50.1%).

### Preparation 14: 2,2-difluoro-2-(2-fluorophenoxy)acetic acid

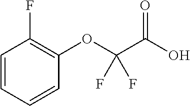 [SMILES: O=C(O)C(F)(F)Oc1ccccc1F]

The title compound was prepared like PREPARATION 13, using sodium hydride (254 mg, 5.08 mmol), 2-fluorophenol (427 mg, 3.81 mmol) and sodium 2-bromo-2,2-difluoroacetate (500 mg, 2.54 mmol), and was obtained as an orange oil (143 mg, 27.3%). ¹⁹F NMR (376 MHz, DMSO-d₆) δ ppm −130.44--128.88 (m, 1F), −77.03 (br s, 2F).

### Preparation 15: 2,2-difluoro-2-(3-fluorophenoxy)acetic acid

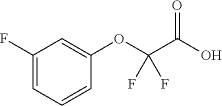 [SMILES: O=C(O)C(F)(F)Oc1cccc(F)c1]

The title compound was prepared like PREPARATION 13 using sodium hydride (60 wt %, 203 mg, 5.08 mmol), 3-fluorophenol (427 mg, 3.81 mmol) and sodium 2-bromo-2,2-difluoroacetate (500 mg, 2.54 mmol), and was obtained as an orange oil (93 mg, 18%).

### Preparation 16: 2,2-difluoro-2-(o-tolyloxy)acetic acid

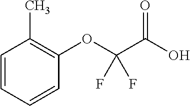

The title compound was prepared like PREPARATION 13, using sodium hydride (60 wt %, 254 mg, 6.35 mmol), o-cresol (412 mg, 3.81 mmol) and sodium 2-bromo-2,2-difluoroacetate (500 mg, 2.54 mmol), and was obtained as an orange oil (272 mg, 53%).

### Preparation 17: 2,2-difluoro-2-(m-tolyloxy)acetic acid

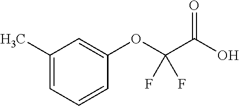

The title compound was prepared like PREPARATION 13, using sodium hydride (60 wt %, 254 mg, 5.08 mmol), m-cresol (412 mg, 3.81 mmol) and sodium 2-bromo-2,2-difluoroacetate (500 mg, 2.54 mmol), and was obtained as an orange oil (513 mg).

### Example 1: (R)-1-(3-methylbenzyl)-N-(1-methylpyrrolidin-3-yl)cyclopropane-1-carboxamide

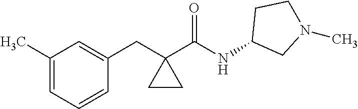

A solution of 1-(3-methylbenzyl)cyclopropanecarboxylic acid (76 mg, 0.40 mmol) in DMF (2 mL) was treated with Et₃N (56 μL, 0.40 mmol) and (R)-1-methylpyrrolidin-3-amine (40 mg, 0.40 mmol). After stirring for 5 minutes, HATU (152 mg, 0.400 mmol) was added. The reaction mixture was stirred at room temperature overnight and was then filtered through a hydrophilic PTFE 0.45 m Millipore® filter, rinsing with MeOH. The product was purified by preparative HPLC (Phenomenex Gemini® NX—C18, 5 μm, ID 30 mm×150 mm) using a gradient of 10-100% water/ACN in water (Basic Mode) to give the title compound as a yellow oil (51.6 mg, 47%). ¹H NMR (500 MHz, CD₃OD) δ ppm 0.75 (s, 2H), 1.05-1.15 (m, 2H), 1.43-1.54 (m, 1H), 2.15 (dtd, J=13.6, 8.4, 5.6 Hz, 1H), 2.22-2.29 (m, 1H), 2.30 (s, 6H), 2.35-2.46 (m, 1H), 2.59-2.69 (m, 2H), 2.88-3.00 (m, 2H), 4.24-4.33 (m, 1H), 7.01 (d, J=7.3 Hz, 1H), 7.04 (d, J=7.8 Hz, 1H), 7.08 (s, 1H), 7.12-7.17 (m, 1H); ESI-MS m/z [M+H]⁺ 273.3.

### Example 2: N-(1-methyl-4-phenylpyrrolidin-3-yl)-1-(3-methylbenzyl)cyclopropane-1-carboxamide

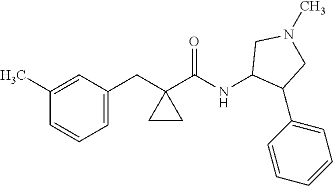 [SMILES: Cc1cccc(CC2(C(=O)NC3CN(C)CC3c3ccccc3)CC2)c1]

To a vial containing 1-(3-methylbenzyl)cyclopropanecarboxylic acid (0.075 g, 0.394 mmol) in DMF (3 mL) were added 1-methyl-4-phenylpyrrolidin-3-amine (0.069 g, 0.394 mmol), DIPEA (171 μL, 0.986 mmol) and HATU (0.180 g, 0.473 mmol). The mixture was stirred at room temperature overnight and was then filtered through a hydrophilic PTFE 0.45 m Millipore® filter, rinsing with methanol. The product was purified by preparative HPLC (Phenomenex Gemini C18, 5 μm, ID 30 mm×150 mm column) using a gradient of 10-100% ACN in water (Acid Mode) to give a TFA salt of the title compound as a pale yellow oil (64 mg, 35%). ¹H NMR (400 MHz, CD₃OD) δ ppm 0.45-0.62 (m, 2H), 0.75-0.83 (m, 1H), 1.13 (d, J=2.0 Hz, 1H), 1.30-1.44 (m, 1H), 2.22-2.33 (m, 3H), 2.55-2.94 (m, 2H), 2.95-3.08 (m, 3H), 3.08-3.23 (m, 1H), 3.48-3.82 (m, 2H), 3.84-4.38 (m, 2H), 6.80-6.91 (m, 1H), 6.93-7.06 (m, 2H), 7.06-7.21 (m, 3H), 7.24-7.36 (m, 3H); ESI-MS m/z [M+H]⁺ 349.5.

### Example 3: N-(trans-4-isopropyl-1-methylpyrrolidin-3-yl)-1-(3-methylbenzyl)cyclopropane-1-carboxamide

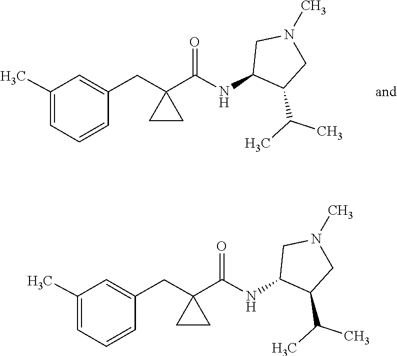

To a 20 mL vial charged with DMF (2 mL) were added 1-(3-methylbenzyl)cyclopropanecarboxylic acid (50.0 mg, 0.263 mmol) and 2-chloro-1-methylpyridin-1-ium iodide (201 mg, 0.788 mmol). The reaction mixture was stirred for 30 minutes. Next, trans-4-isopropyl-1-methylpyrrolidin-3-amine (37.4 mg, 0.263 mmol) and Et₃N (0.183 mL, 1.31 mmol) were added, and the reaction was stirred at room temperature overnight. The product was purified by preparative HPLC (Phenomenex Gemini® NX—C18, 5 μm, ID 30 mm×150 mm) using a gradient of 10-100% water/ACN in water (Basic Mode) to give the title compound as a colorless oil (8 mg, 11%). ¹H NMR (400 MHz, CD₃OD) δ ppm 0.66-0.75 (m, 3H), 0.85 (d, J=6.3 Hz, 3H), 1.55-1.69 (m, 1H), 1.72-1.79 (m, 6H), 2.14-2.22 (m, 1H), 2.77-2.91 (m, 1H), 2.95 (br s, 3H), 3.47-3.71 (m, 1H), 3.73-3.96 (m, 2H), 4.08 (s, 3H), 7.17 (t, J=7.5 Hz, 1H), 7.44 (t, J=7.3 Hz, 1H), 7.55 (d, J=8.8 Hz, 1H), 7.63-7.78 (m, 1H), 7.88 (br s, 1H); ESI-MS m/z [M+H]⁺ 315.4.

### Example 4: N-(1,3-dimethylpiperidin-4-yl)-1-(3-methylbenzyl)cyclopropane-1-carboxamide

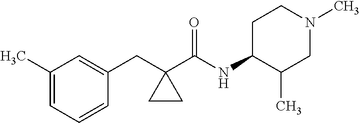

To a 20 mL vial charged with DMF (8 mL) were added 1-(3-methylbenzyl)cyclopropanecarboxylic acid (300 mg, 1.58 mmol) and HATU (899 mg, 2.36 mmol) followed by 1,3-dimethylpiperidin-4-amine (202 mg, 1.58 mmol) and Et₃N (0.440 mL, 3.15 mmol). The reaction mixture was stirred at room temperature overnight and was then purified by preparative HPLC (Phenomenex Gemini® NX—C18, 5 μm, ID 30 mm×100 mm) using a gradient of 10-100% ACN in water (Acid Mode) to give a TFA salt of the title compound as a yellow solid (267 mg, 41%). ¹H NMR (400 MHz, CD₃OD) δ ppm 0.70-0.96 (m, 5H), 1.09-1.33 (m, 2H), 1.67-2.19 (m, 3H), 2.31-2.38 (m, 3H), 2.41-2.50 (m, 1H), 2.61 (td, J=13.3, 3.4 Hz, 1H), 2.77-2.99 (m, 4H), 3.03-3.23 (m, 2H), 3.40-3.65 (m, 2H), 4.03-4.16 (m, 1H), 7.03-7.29 (m, 4H); ESI-MS m/z [M+H]⁺ 301.4.

### Example 5: N-((3S,4S)-3-fluoropiperidin-4-yl)-1-(3-methylbenzyl)cyclopropane-1-carboxamide

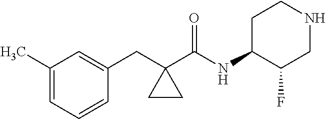 [SMILES: Cc1cccc(CC2(C(=O)N[C@H]3CCNC[C@@H]3F)CC2)c1]

### Step A: tert-butyl (3S,4S)-3-fluoro-4-(1-(3-methylbenzyl)cyclopropane-1-carboxamido)piperidine-1-carboxylate

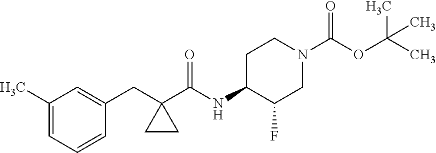 [SMILES: Cc1cccc(CC2(C(=O)N[C@H]3CCN(C(=O)OC(C)(C)C)C[C@@H]3F)CC2)c1]

To a solution of 1-(3-methylbenzyl)cyclopropane-1-carboxylic acid (150 mg, 0.788 mmol) in DMA (3.94 mL) were added DIPEA (413 μL, 2.36 mmol) and HATU (450 mg, 1.18 mmol). After stirring for 5 minutes at room temperature, tert-butyl (3S,4S)-4-amino-3-fluoropiperidine-1-carboxylate (189 mg, 0.867 mmol) was added. The reaction mixture was stirred at room temperature overnight and was then diluted with saturated aqueous NH₄Cl and extracted with DCM (2×). The combined organic layers were washed with saturated aqueous NaHCO₃ followed by saturated aqueous NaCl, dried over Na₂SO₄, filtered, and concentrated in vacuo to give the title compound (308 mg).

### Step B: N-((3S,4S)-3-fluoropiperidin-4-yl)-1-(3-methylbenzyl)cyclopropane-1-carboxamide

To crude tert-butyl (3S,4S)-3-fluoro-4-(1-(3-methylbenzyl)cyclopropane-1-carboxamido)piperidine-1-carboxylate (308 mg, 0.789 mmol) was added TFA (1 mL, 13 mmol). The reaction mixture was stirred at room temperature overnight and then concentrated in vacuo. The residue was taken up in MeOH, filtered through a hydrophilic PTFE 0.45 m Millipore® filter and purified by preparative HPLC (Phenomenex Gemini® NX—C18, 5 μm, ID 30 mm×150 mm) using a gradient of 10-70% water/ACN in water (Basic Mode). The product-containing fractions were lyophilized to give the title compound as a tan semisolid (16.8 mg, 7.3% over two steps). ESI-MS m/z [M+H]⁺ 291.20.

### Example 6: N-(1,4-dimethylpyrrolidin-3-yl)-1-(3-methylbenzyl)cyclopropane-1-carboxamide

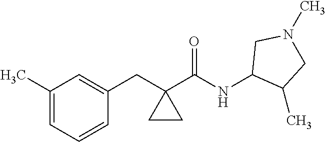

To a solution of 1-(3-methylbenzyl)cyclopropane-1-carboxylic acid (150 mg, 0.788 mmol) in DMA (3.94 mL) were added DIPEA (413 μL, 2.36 mmol) and HATU (450 mg, 1.18 mmol). After stirring for 5 minutes, 1,4-dimethylpyrrolidin-3-amine (90 mg, 0.788 mmol) was added. The reaction mixture was stirred at room temperature overnight and then diluted with saturated aqueous NaHCO₃ and extracted with DCM (3×). The combined organic layers were washed with saturated aqueous NaCl, dried over Na₂SO₄, filtered, and concentrated in vacuo. The residue was taken up in MeOH, filtered through a hydrophilic PTFE 0.45 m Millipore® filter and purified by preparative HPLC (Phenomenex Gemini® NX—C18, 5 μm, ID 30 mm×150 mm) using a gradient of 30-80% water/ACN in water (Basic Mode). The title compound was obtained as a yellow oil (46.1 mg, 20%). ¹H NMR (500 MHz, CDCl₃) δ ppm 0.55 (d, J=7.1 Hz, 1.8H), 0.68-0.84 (m, 2H), 0.95 (d, J=6.8 Hz, 1.2H), 1.17-1.37 (m, 2H), 1.65-1.78 (m, 0.8H), 2.00 (dd, J=9.3, 6.8 Hz, 0.6H), 2.07 (dd, J=9.8, 3.7 Hz, 0.6H), 2.21 (s, 3H), 2.31 (d, J=1.7 Hz, 3H), 2.39-3.01 (m, 5H), 3.82-3.91 (m, 0.4H), 4.33-4.43 (m, 0.6H), 5.88 (br d, J=8.8 Hz, 0.6H), 5.99 (br d, J=7.6 Hz, 0.4H), 6.99-7.22 (m, 4H); ESI-MS m/z [M+H]⁺ 287.20.

### Example 7: 1-(2-chlorophenoxy)-N-(trans-3-ethyl-1-methylpiperidin-4-yl)cyclopropane-1-carboxamide

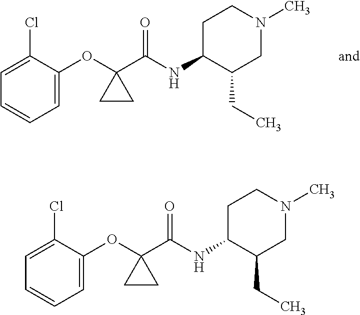 [SMILES: CC[C@H]1CN(C)CC[C@@H]1NC(=O)C1(Oc2ccccc2Cl)CC1 ; "and" ; CC[C@@H]1CN(C)CC[C@H]1NC(=O)C1(Oc2ccccc2Cl)CC1]

In an 8 mL vial were combined 1-(2-chlorophenoxy)cyclopropane-1-carboxylic acid (0.06 g, 0.282 mmol), trans-3-ethyl-1-methylpiperidin-4-amine (0.040 g, 0.282 mmol), HATU (0.107 g, 0.282 mmol) and DIPEA (0.147 mL, 0.847 mmol) in DMF (2 mL) to give a yellow solution. The reaction mixture was stirred at room temperature overnight and then purified by preparative HPLC (Phenomenex Gemini® NX—C18, 5 μm, ID 30 mm×150 mm) using a gradient of 10-100% water/ACN in water (Basic Mode) to give the title compound as a light brown film (58.8 mg, 62%). ¹H NMR (500 MHz, CD₃OD) δ ppm 0.74-0.80 (m, 3H), 0.93-1.04 (m, 1H), 1.12-1.26 (m, 2H), 1.34-1.42 (m, 1H), 1.45-1.57 (m, 3H), 1.58-1.65 (m, 1H), 1.74 (s, 2H), 1.99-2.10 (m, 1H), 2.27 (s, 3H), 2.79-2.96 (m, 2H), 3.48-3.59 (m, 1H), 6.96-7.09 (m, 2H), 7.21-7.29 (m, 1H), 7.36-7.43 (m, 1H); ESI-MS m/z [M+H]⁺ 337.3.

### Example 8: 1-(2-chlorophenoxy)-N-(1,2-dimethylpiperidin-4-yl)cyclopropane-1-carboxamide

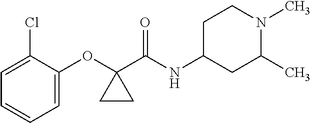 [SMILES: CC1CC(NC(=O)C2(Oc3ccccc3Cl)CC2)CCN1C]

A solution of 1-(2-chlorophenoxy)cyclopropane-1-carboxylic acid (0.020 g, 0.094 mmol), DIPEA (0.066 mL, 0.376 mmol), 2-chloro-1-methylpyridinium iodide (0.029 g, 0.113 mmol), and NMP (0.5 mL) was stirred at 45° C. for 60 minutes and then 1,2-dimethylpiperidin-4-amine (0.012 g, 0.094 mmol) was added. The solution was stirred at 45° C. overnight. The sample was purified by preparative HPLC (Phenomenex Gemini® NX—C18, 5 μm, ID 30 mm×100 mm) using a gradient of 10-60% ACN in water (Acid Mode). The product-containing fractions were collected and re-purified by preparative HPLC (Phenomenex Gemini® NX—C18, 5 μm, ID 30 mm×150 mm) using a gradient of 10-90% water/ACN in water (Basic Mode) to give the title compound (6.1 mg, 20%). ESI-MS m/z [M+H]⁺ 323.1.

### Example 9: 1-(2-chlorophenoxy)-N-(hexahydro-1H-pyrrolizin-1-yl)cyclopropane-1-carboxamide

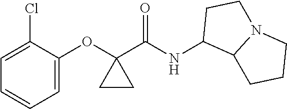 [SMILES: O=C(NC1CCN2CCCC12)C1(Oc2ccccc2Cl)CC1]

To a 4 mL vial equipped with stir bar and charged with a solution of hexahydro-1H-pyrrolizin-1-amine (0.012 g, 0.094 mmol), DIPEA (0.049 mL, 0.282 mmol) and 1-(2-chlorophenoxy)cyclopropane-1-carboxylic acid (20 mg, 0.094 mmol) in DMA (0.5 mL) was added T3P (0.140 mL, 0.235 mmol). The reaction mixture was stirred for 18 hours at 45° C. Methanol (0.5 mL) was added and the solution was purified by preparative HPLC (Phenomenex Gemini® NX—C18, 5 μm, ID 30 mm×100 mm) using a gradient of 10-50% ACN in water (Acid Mode). Lyophilization of the pure fractions gave a TFA salt of the title compound (8.9 mg, 22%). ¹H NMR (400 MHz, CD₃OD) δ ppm 1.14-1.30 (m, 2H), 1.49-1.66 (m, 2H), 1.66-1.75 (m, 0.4H), 1.80-1.92 (m, 1H), 1.95-2.01 (m, 1H), 2.01-2.13 (m, 2H), 2.15-2.27 (m, 2H), 2.32-2.41 (m, 0.6H), 2.98 (ddd, J=11.5, 9.6, 6.1 Hz, 0.4H), 3.15-3.29 (m, 1.6H), 3.45-3.55 (m, 1H), 3.65-3.76 (m, 1H), 4.01-4.08 (m, 0.6H), 4.28-4.39 (m, 1H), 4.53-4.61 (m, 0.4H), 6.98-7.07 (m, 2H), 7.23-7.29 (m, 1H), 7.40 (ddd, J=7.9, 4.8, 1.5 Hz, 1H), 8.38 (br dd, J=19.3, 6.0 Hz, 1H); ESI-MS m/z [M+H]⁺ 321.1.

### Example 10: N-(trans-1,3-dimethylpiperidin-4-yl)-1-(4-fluorobenzyl)cyclopropane-1-carboxamide

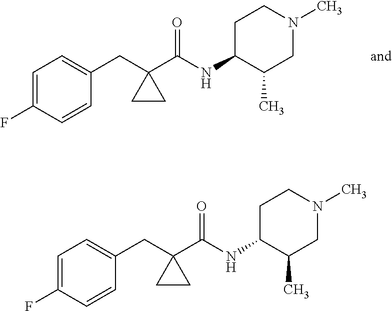 [SMILES: C[C@H]1CN(C)CC[C@@H]1NC(=O)C1(Cc2ccc(F)cc2)CC1 ; "and" ; C[C@@H]1CN(C)CC[C@H]1NC(=O)C1(Cc2ccc(F)cc2)CC1]

A solution of 1-(4-fluorobenzyl)cyclopropane-1-carboxylic acid (69 mg, 0.355 mmol), trans-1,3-dimethylpiperidin-4-amine (71.9 mg, 0.533 mmol), HATU (207 mg, 0.533 mmol) and DIPEA (248 μL, 1.42 mmol) in THE (1.78 mL) was stirred at room temperature overnight. The reaction mixture was then diluted with MeOH, filtered through a hydrophilic PTFE 0.45 m Millipore® filter and purified by preparative HPLC (Phenomenex Gemini® NX—C18, 5 μm, ID 30 mm×150 mm) using a gradient of 10-100% water/ACN in water (Basic Mode). A TFA salt of the title compound was obtained as an orange solid (118 mg, 79%). ¹H NMR (400 MHz, CDCl₃) δ ppm 0.64 (d, J=6.5 Hz, 3H), 0.76-0.89 (m, 2H), 1.19-1.28 (m, 1H), 1.32-1.41 (m, 1H), 1.71-1.86 (m, 1H), 1.89-2.01 (m, 2H), 2.32-2.43 (m, 1H), 2.63-2.73 (m, 1H), 2.76 (s, 3H), 2.84 (d, J=16.3 Hz, 1H), 3.05 (d, J=16.3 Hz, 1H), 3.37-3.44 (m, 1H), 3.49-3.56 (m, 1H), 3.58-3.69 (m, 1H), 5.41 (br d, J=8.5 Hz, 1H), 6.96-7.05 (m, 2H), 7.26-7.30 (m, 2H), 13.00-13.24 (m, 1H); ESI-MS m/z [M+H]⁺ 305.3.

### Example 11: N-(trans-1,3-dimethylpiperidin-4-yl)-1-(4-methylbenzyl)cyclopropane-1-carboxamide

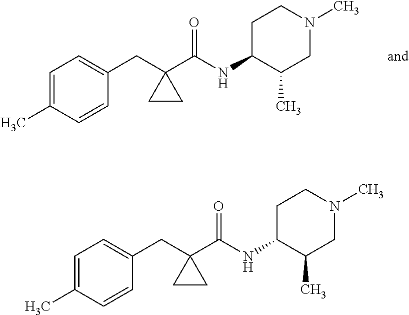

A TFA salt of the title compound was prepared like EXAMPLE 10, using 1-(4-methylbenzyl)cyclopropane-1-carboxylic acid (57 mg, 0.300 mmol), and was obtained as an orange solid (72 mg, 58%). ¹H NMR (400 MHz, CDCl₃) δ ppm 0.62 (d, J=6.53 Hz, 3H), 0.77-0.83 (m, 1H), 0.84-0.91 (m, 1H), 1.25 (ddd, J=9.7, 6.1, 3.5 Hz, 1H), 1.33-1.40 (m, 1H), 1.62-1.74 (m, 1H), 1.78-1.89 (m, 1H), 1.94 (dq, J=14.1, 3.2 Hz, 1H), 2.32 (s, 3H), 2.33-2.40 (m, 1H), 2.64-2.72 (m, 1H), 2.75 (s, 3H), 2.82 (d, J=16.1 Hz, 1H), 3.02 (d, J=16.1 Hz, 1H), 3.37-3.44 (m, 1H), 3.48-3.55 (m, 1H), 3.55-3.67 (m, 1H), 5.46 (br d, J=8.8 Hz, 1H), 7.10-7.16 (m, 2H), 7.18-7.24 (m, 2H) 12.73-12.87 (m, 1H); ESI-MS m/z [M+H]⁺ 301.4.

### Example 12: N-(trans-1,3-dimethylpiperidin-4-yl)-1-(2-methylbenzyl)cyclopropane-1-carboxamide

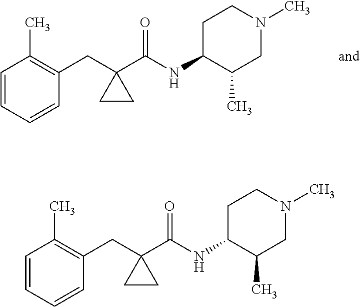 [SMILES: Cc1ccccc1CC1(C(=O)N[C@H]2CCN(C)C[C@@H]2C)CC1 ; "and" ; Cc1ccccc1CC1(C(=O)N[C@@H]2CCN(C)C[C@H]2C)CC1]

A TFA salt of the title compound was prepared like EXAMPLE 10, using 1-(2-methylbenzyl)cyclopropane-1-carboxylic acid (55 mg, 0.289 mmol), and was obtained as an orange solid (82 mg, 68%). ¹H NMR (400 MHz, CDCl₃) δ ppm 0.71 (d, J=6.8 Hz, 3H), 0.73-0.81 (m, 2H), 1.27-1.31 (m, 1H), 1.37-1.43 (m, 1H), 1.66-1.79 (m, 1H), 1.83-1.94 (m, 1H), 1.98 (dq, J=14.1, 3.2 Hz, 1H), 2.32 (s, 3H), 2.33-2.43 (m, 1H), 2.64-2.72 (m, 1H), 2.76 (s, 3H), 2.81 (d, J=16.8 Hz, 1H), 3.07 (d, J=16.8 Hz, 1H), 3.38-3.45 (m, 1H), 3.49-3.57 (m, 1H), 3.63-3.73 (m, 1H), 5.42 (br d, J=8.3 Hz, 1H), 7.10-7.17 (m, 2H), 7.17-7.21 (m, 1H), 7.30-7.35 (m, 1H), 12.81-12.98 (m, 1H); ESI-MS m/z [M+H]⁺ 301.4.

### Example 13: 1-(4-fluorobenzyl)-N-((3S,4S)-3-methylpiperidin-4-yl)cyclopropane-1-carboxamide

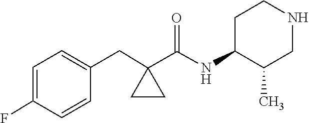 [SMILES: C[C@H]1CNCC[C@@H]1NC(=O)C1(Cc2ccc(F)cc2)CC1]

### Step A: tert-butyl (3S,4S)-4-(1-(4-fluorobenzyl)cyclopropane-1-carboxamido)-3-methylpiperidine-1-carboxylate

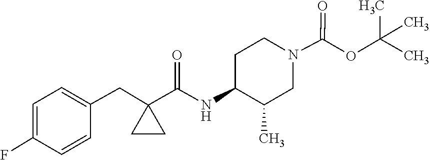 [SMILES: C[C@H]1CN(C(=O)OC(C)(C)C)CC[C@@H]1NC(=O)C1(Cc2ccc(F)cc2)CC1]

A solution of 1-(4-fluorobenzyl)cyclopropane-1-carboxylic acid (101 mg, 0.520 mmol), tert-butyl (3S,4S)-4-amino-3-methylpiperidine-1-carboxylate (145 mg, 0.676 mmol), HATU (262 mg, 0.676 mmol) and Et₃N (290 μL, 2.08 mmol) in THF (2.60 mL) was stirred at room temperature for 8 hours. The reaction mixture was then diluted with MeOH, filtered through a Millipore filter and purified by preparative HPLC (Phenomenex Gemini® NX—C18, 5 μm, ID 30 mm×100 mm) using a gradient of 10-100% ACN in water (Acid Mode). The pure fractions were combined and lyophilized to give the title compound as a white solid (170 mg, 84%). ESI-MS m/z [M-t-Bu]⁺ 335.1.

### Step B: 1-(4-fluorobenzyl)-N-((3S,4S)-3-methylpiperidin-4-yl)cyclopropane-1-carboxamide

A solution of tert-butyl (3S,4S)-4-(1-(4-fluorobenzyl)cyclopropane-1-carboxamido)-3-methylpiperidine-1-carboxylate (170 mg, 0.435 mmol) in MeOH (871 μL) and DCM (871 μL) was treated with HCl (653 μL, 2.61 mmol, 4M in dioxane) at room temperature. The reaction mixture stirred at RT for 16 hours and then diluted with MeOH, filtered through a hydrophilic PTFE 0.45 m Millipore® filter and purified by preparative HPLC (Phenomenex Gemini® NX—C18, 5 μm, ID 30 mm×100 mm) using a gradient of 10-100% ACN in water (Acid Mode). The pure fractions were combined and lyophilized to give a TFA salt of the title compound as a white solid (140 mg, 80%). ¹H NMR (400 MHz, CDCl₃) δ ppm 0.66 (d, J=6.8 Hz, 3H), 0.76-0.91 (m, 2H), 1.24-1.30 (m, 1H), 1.34-1.42 (m, 1H), 1.46-1.58 (m, 1H), 1.61-1.74 (m, 1H), 1.89-1.99 (m, 1H), 2.45-2.59 (m, 1H), 2.77-2.96 (m, 3H), 2.99-3.08 (m, 1H), 3.21-3.28 (m, 1H), 3.33 (br d, J=12.3 Hz, 1H), 3.59-3.72 (m, 1H), 5.36 (d, J=8.8 Hz, 1H), 6.98-7.05 (m, 2H), 7.27-7.33 (m, 2H), 9.04-9.21 (m, 1H), 9.47-9.59 (m, 1H); ESI-MS m/z [M+H]⁺ 291.3.

### Example 14: 2,2-difluoro-2-phenoxy-N-(piperidin-4-yl)acetamide

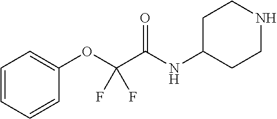 [SMILES: O=C(NC1CCNCC1)C(F)(F)Oc1ccccc1]

### Step A: tert-butyl 4-(2,2-difluoro-2-phenoxyacetamido)piperidine-1-carboxylate

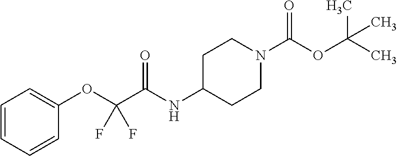 [SMILES: CC(C)(C)OC(=O)N1CCC(NC(=O)C(F)(F)Oc2ccccc2)CC1]

The title compound was prepared like STEP A of EXAMPLE 13, using 2,2-difluoro-2-phenoxyacetic acid (200 mg, 1.06 mmol), tert-butyl 4-aminopiperidine-1-carboxylate (319 mg, 1.59 mmol), HATU (606 mg, 1.59 mmol) and Et₃N (596 μL, 4.25 mmol) in DMA (5.32 mL), and was obtained as a white solid (176 mg, 45%). ESI-MS m/z [M-t-Bu]⁺315.3.

### Step B: 2,2-difluoro-2-phenoxy-N-(piperidin-4-yl)acetamide

A solution of tert-butyl 4-(2,2-difluoro-2-phenoxyacetamido)piperidine-1-carboxylate (176 mg, 0.475 mmol) in DCM (1.2 mL) was treated with HCl in dioxane (4 M, 1.19 mL, 4.75 mmol) dropwise via syringe at room temperature. The resulting mixture was stirred at RT for 16 hours and then diluted with MeOH (1.5 mL), filtered through a hydrophilic PTFE 0.45 m Millipore® filter and purified by preparative HPLC (Phenomenex Gemini® NX—C18, 5 μm, ID 30 mm×100 mm) using a gradient of 10-100% ACN in water (Acid Mode). The pure fractions were combined and lyophilized to give a TFA salt of the title compound as a white solid (119 mg, 65%). ¹H NMR (400 MHz, DMSO-d₆) δ ppm 1.59-1.79 (m, 2H), 1.87 (br dd, J=13.4, 2.6 Hz, 2H), 2.99 (br t, J=11.9 Hz, 2H), 3.29 (br d, J=12.8 Hz, 3H), 3.92 (dtd, J=11.2, 7.3, 4.0 Hz, 1H), 7.23-7.28 (m, 2H), 7.29-7.38 (m, 1H), 7.42-7.51 (m, 2H), 9.19 (br d, J=7.8 Hz, 1H); ESI-MS m/z [M+H]⁺ 271.3.

### Example 15: 1-(3-chlorobenzyl)-N-((3S,4S)-3-methylpiperidin-4-yl)cyclopropane-1-carboxamide

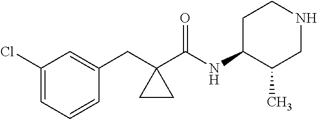 [SMILES: C[C@H]1CNCC[C@@H]1NC(=O)C1(Cc2cccc(Cl)c2)CC1]

### Step A: tert-butyl (3S,4S)-4-(1-(3-chlorobenzyl)cyclopropane-1-carboxamido)-3-methylpiperidine-1-carboxylate

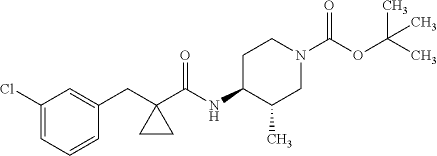 [SMILES: C[C@H]1CN(C(=O)OC(C)(C)C)CC[C@@H]1NC(=O)C1(Cc2cccc(Cl)c2)CC1]

A solution of 1-(3-chlorobenzyl)cyclopropane-1-carboxylic acid (56 mg, 0.266 mmol, 1 eq), tert-butyl (3S,4S)-4-amino-3-methylpiperidine-1-carboxylate (68.4 mg, 0.319 mmol, 1.2-1.5 eq), HATU (121 mg, 0.319 mmol, 1.2-1.5 eq) and Et₃N (148 μL, 1.06 mmol, 4-6 eq) in THE (1.33 mL, 0.20 M) was stirred at room temperature for 16 hours. The reaction mixture was then diluted with MeOH (1 mL), filtered through a hydrophilic PTFE 0.45 m Millipore® filter and purified by preparative HPLC (Phenomenex Gemini® NX—C18, 5 μm, ID 30 mm×100 mm) using a gradient of 10-100% ACN in water (Acid Mode). The pure fractions were combined and lyophilized to give the title compound as a white solid (92 mg, 85%). ESI-MS m/z [M+H]⁺ 407.4.

### Step B: 1-(3-chlorobenzyl)-N-((3S,4S)-3-methylpiperidin-4-yl)cyclopropane-1-carboxamide

A solution of tert-butyl (3S,4S)-4-(1-(3-chlorobenzyl)cyclopropane-1-carboxamido)-3-methylpiperidine-1-carboxylate (92 mg, 0.226 mmol, 1 eq) in MeOH (452 μL) and DCM (452 μL) was treated with HCl in dioxane (4 M, 339 μL, 1.36 mmol, 6-10 eq) at room temperature. The reaction mixture was stirred at RT for 16 hours and was then diluted with MeOH (1 mL), filtered through a hydrophilic PTFE 0.45 m Millipore® filter and purified by preparative HPLC (Phenomenex Gemini® NX—C18, 5 μm, ID 30 mm×100 mm) using a gradient of 10-100% ACN in water (Acid Mode). The pure fractions were combined and lyophilized to give a TFA salt of the title compound as a white solid (84 mg, 88%). ¹H NMR (400 MHz, CDCl₃) δ ppm 0.68 (d, J=6.8 Hz, 3H), 0.79-0.90 (m, 2H), 1.22-1.31 (m, 1H), 1.34-1.42 (m, 1H), 1.47-1.60 (m, 1H), 1.63-1.76 (m, 1H), 1.92-2.01 (m, 1H), 2.47-2.59 (m, 1H), 2.85 (br d, J=16.3 Hz, 2H), 3.04 (d, J=16.6 Hz, 1H), 3.21-3.30 (m, 1H), 3.34 (br d, J=12.0 Hz, 1H), 3.62-3.74 (m, 1H), 5.35 (d, J=8.8 Hz, 1H), 7.17-7.26 (m, 3H), 7.32-7.35 (m, 1H), 9.05-9.22 (m, 1H), 9.42-9.55 (m, 1H); ESI-MS m/z [M+H]⁺ 307.3.

### Example 16: 1-(2-methylbenzyl)-N-((3S,4S)-3-methylpiperidin-4-yl)cyclopropane-1-carboxamide

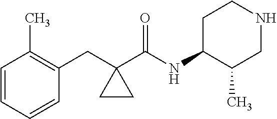 [SMILES: Cc1ccccc1CC1(C(=O)N[C@H]2CCNC[C@@H]2C)CC1]

### Step A: tert-butyl (3S,4S)-3-methyl-4-(1-(2-methylbenzyl)cyclopropane-1-carboxamido)piperidine-1-carboxylate

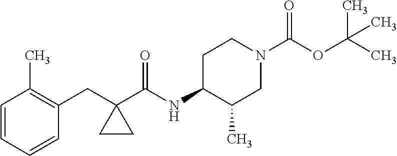 [SMILES: Cc1ccccc1CC1(C(=O)N[C@H]2CCN(C(=O)OC(C)(C)C)C[C@@H]2C)CC1]

The title compound was prepared like STEP A in EXAMPLE 15, using 1-(2-methylbenzyl)cyclopropane-1-carboxylic acid (88 mg, 0.0.346 mmol), tert-butyl (3S,4S)-4-amino-3-methylpiperidine-1-carboxylate (129 mg, 0.601 mmol), HATU (233 mg, 0.601 mmol) and Et₃N (258 μL, 1.85 mmol) in THF (2.31 mL), and was obtained as a white solid (143 mg, 80%). ESI-MS m/z [M-t-Bu]⁺331.2.

### Step B: 1-(2-methylbenzyl)-N-((3S,4S)-3-methylpiperidin-4-yl)cyclopropane-1-carboxamide

A TFA salt of the title compound was prepared like STEP B in EXAMPLE 15, using tert-butyl (3S,4S)-3-methyl-4-(1-(2-methylbenzyl)cyclopropane-1-carboxamido)piperidine-1-carboxylate (143 mg, 0.370 mmol), and was obtained as a white solid (127 mg, 86%). ¹H NMR (400 MHz, CDCl₃) δ ppm 0.72 (d, J=6.5 Hz, 3H), 0.74-0.83 (m, 2H), 1.29-1.36 (m, 1H), 1.39-1.46 (m, 1H), 1.46-1.55 (m, 1H), 1.59-1.72 (m, 1H), 1.99 (br dd, J=14.0, 2.0 Hz, 1H), 2.31 (s, 3H), 2.47-2.59 (m, 1H), 2.77-2.93 (m, 2H), 3.05 (d, J=17.1 Hz, 1H), 3.21-3.28 (m, 1H), 3.33 (br d, J=12.8 Hz, 1H), 3.62-3.76 (m, 1H), 4.25-4.45 (m, 1H), 5.41 (d, J=8.5 Hz, 1H), 7.12-7.16 (m, 2H), 7.17-7.21 (m, 1H), 7.33-7.39 (m, 1H), 8.93-9.09 (m, 1H), 9.24-9.36 (m, 1H); ESI-MS m/z [M+H]⁺ 287.4.

### Example 17: 1-(2-chlorobenzyl)-N-(trans-1,3-dimethylpiperidin-4-yl)cyclopropane-1-carboxamide

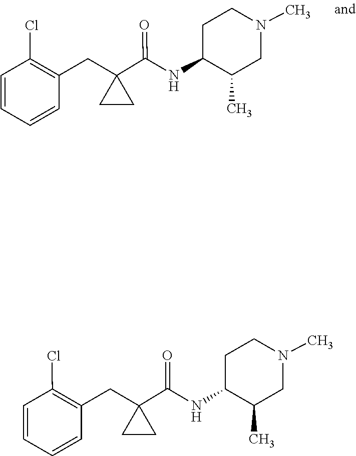 [SMILES: C[C@H]1CN(C)CC[C@@H]1NC(=O)C1(Cc2ccccc2Cl)CC1 ; C[C@@H]1CN(C)CC[C@H]1NC(=O)C1(Cc2ccccc2Cl)CC1]

A TFA salt of the title compound was prepared like STEP A of EXAMPLE 15, using 1-(2-chlorobenzyl)cyclopropane-1-carboxylic acid (50.0 mg, 0.237 mmol), trans-1,3-dimethylpiperidin-4-amine (39.6 mg, 0.309 mmol), HATU (120 mg, 0.309 mmol) and Et₃N (132 μL, 0.949 mmol) in THE (1.19 mL), and was obtained as a white solid (85 mg, 82%). ¹H NMR (400 MHz, CDCl₃) δ ppm 0.72 (d, J=6.5 Hz, 3H), 0.79-0.87 (m, 2H), 1.24-1.30 (m, 1H), 1.37-1.42 (m, 1H), 1.70-1.84 (m, 1H), 1.92-2.00 (m, 2H), 2.32-2.42 (m, 1H), 2.64-2.73 (m, 1H), 2.76 (s, 3H), 2.97 (d, J=16.8 Hz, 1H), 3.22 (d, J=17.1 Hz, 1H), 3.40-3.47 (m, 1H), 3.54 (dt, J=12.2, 2.1 Hz, 1H), 3.62-3.73 (m, 1H), 5.53 (br d, J=8.8 Hz, 1H), 7.16-7.24 (m, 2H), 7.38-7.46 (m, 2H); ESI-MS m/z [M+H]⁺ 321.1.

### Example 18: 1-(4-chlorobenzyl)-N-((3S,4S)-3-methylpiperidin-4-yl)cyclopropane-1-carboxamide

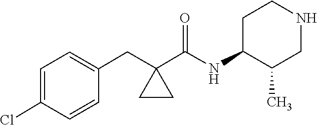 [SMILES: C[C@H]1CNCC[C@@H]1NC(=O)C1(Cc2ccc(Cl)cc2)CC1]

A TFA salt of the title compound was prepared like EXAMPLE 15, using 1-(4-chlorobenzyl)cyclopropane-1-carboxylic acid (77 mg, 0.366 mmol), and was obtained as a white solid (97 mg, 63% over two steps). ¹H NMR (400 MHz, CDCl₃) δ ppm 0.66 (d, J=6.5 Hz, 3H), 0.77-0.89 (m, 2H), 1.22-1.30 (m, 1H), 1.37 (ddd, J=9.8, 5.8, 3.5 Hz, 1H), 1.48-1.61 (m, 1H), 1.63-1.77 (m, 1H), 1.91-1.99 (m, 1H), 2.52 (q, J=11.5 Hz, 1H), 2.79-2.92 (m, 2H), 3.00-3.06 (m, 1H), 3.22-3.30 (m, 1H), 3.30-3.44 (m, 2H), 3.61-3.72 (m, 1H), 5.37 (br d, J=8.8 Hz, 1H), 7.24-7.31 (m, 4H), 9.03-9.17 (m, 1H), 9.43-9.56 (m, 1H); ESI-MS m/z [M+H]⁺ 307.3.

### Example 19: 3-(4-chlorophenyl)-2,2-dimethyl-N-((3S,4S)-3-methylpiperidin-4-yl)propanamide

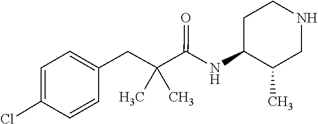 [SMILES: C[C@H]1CNCC[C@@H]1NC(=O)C(C)(C)Cc1ccc(Cl)cc1]

A TFA salt of the title compound was prepared like EXAMPLE 15, using 3-(4-chlorophenyl)-2,2-dimethylpropanoic acid (65 mg, 0.306 mmol), and was obtained as a white solid (92 mg, 71% over two steps). ¹H NMR (400 Mlz, CDCl₃) δ ppm 0.84 (d, J=6.8 Hz, 3H), 1.20 (d, J=7.5 Hz, 6H), 1.56-1.69 (m, 1H), 1.80-1.95 (m, 1H), 2.00 (br d, J=13.3 Hz, 1H), 2.54-2.68 (m, 1H), 2.82 (s, 2H), 2.87-3.01 (m, 1H), 3.32-3.38 (m, 1H), 3.42 (br d, J=12.6 Hz, 1H), 3.69-3.83 (m, 1H), 4.15-4.29 (m, 1H), 5.43 (br d, J=8.8 Hz, 1H), 7.05-7.09 (m, 2H), 7.20-7.24 (m, 2H), 9.07-9.24 (m, 1H), 9.27-9.41 (m, 1H); ESI-MS m/z [M+H]⁺ 309.3.

### Example 20: N-(trans-1,3-dimethylpiperidin-4-yl)-1-(3-fluorobenzyl)cyclopropane-1-carboxamide

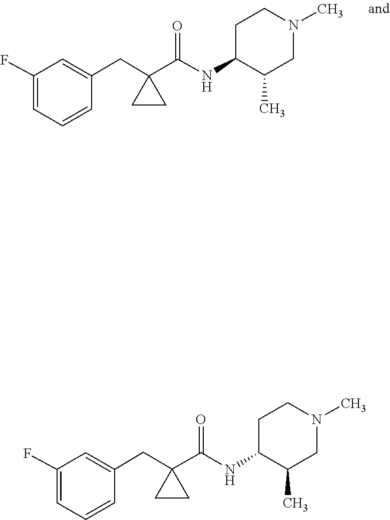 [SMILES: C[C@H]1CN(C)CC[C@@H]1NC(=O)C1(Cc2cccc(F)c2)CC1 ; C[C@@H]1CN(C)CC[C@H]1NC(=O)C1(Cc2cccc(F)c2)CC1]

A TFA salt of the title compound was prepared like STEP A of EXAMPLE 15, using 1-(3-fluorobenzyl)cyclopropane-1-carboxylic acid (62 mg, 0.319 mmol), trans-1,3-dimethylpiperidin-4-amine (51.7 mg, 0.383 mmol), HATU (150 mg, 0.383 mmol) and Et₃N (178 μL, 1.28 mmol) in THE (1.60 mL), and was obtained as a white solid (26 mg, 19%). ¹H NMR (400 MHz, CDCl₃) δ ppm 0.66 (d, J=6.8 Hz, 3H), 0.79-0.90 (m, 2H), 1.21-1.28 (m, 1H), 1.35-1.41 (m, 1H), 1.69-1.82 (m, 1H), 1.87-1.99 (m, 2H), 2.33-2.44 (m, 1H), 2.64-2.74 (m, 1H), 2.77 (s, 3H), 2.86 (d, J=16.6 Hz, 1H), 3.07 (d, J=16.3 Hz, 1H), 3.38-3.46 (m, 1H), 3.49-3.56 (m, 1H), 3.59-3.71 (m, 1H), 5.43 (br d, J=8.5 Hz, 1H), 6.94 (ddd, J=9.2, 7.6, 1.8 Hz, 1H), 7.03-7.10 (m, 2H), 7.24-7.33 (m, 1H), 12.59-12.75 (m, 1H); ESI-MS m/z [M+H]⁺ 305.4.

### Example 21: N-(trans-3-ethyl-1-methylpiperidin-4-yl)-1-(3-methylbenzyl)cyclopropane-1-carboxamide

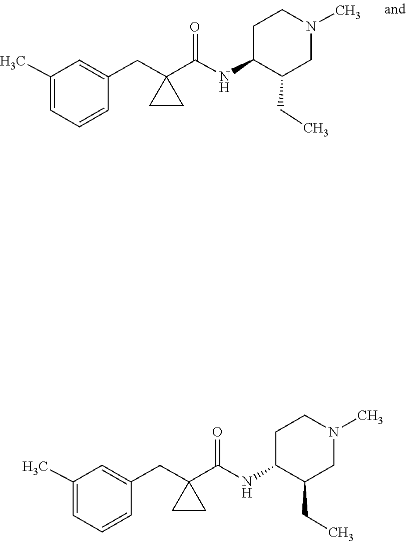

A TFA salt of the title compound was prepared like STEP A of EXAMPLE 15, using 1-(3-methylbenzyl)cyclopropane-1-carboxylic acid (20 mg, 0.105 mmol), trans-3-ethyl-1-methylpiperidin-4-amine (20.5 mg, 0.137 mmol), HATU (53.6 mg, 0.137 mmol) and Et₃N (58.6 μL, 0.421 mmol) in THE (526 μL), and was obtained as a white solid (44 mg, 98%). ¹H NMR (400 MHz, CDCl₃) δ ppm 0.64-0.72 (m, 3H), 0.75-0.85 (m, 2H), 0.86-0.93 (m, 1H), 1.04-1.16 (m, 1H), 1.24 (ddd, J=9.9, 6.4, 3.8 Hz, 1H), 1.38-1.46 (m, 1H), 1.59-1.75 (m, 2H), 1.92-2.00 (m, 1H), 2.29-2.40 (m, 4H), 2.61-2.73 (m, 1H), 2.74-2.82 (m, 4H), 3.06 (d, J=16.6 Hz, 1H), 3.46-3.55 (m, 2H), 3.62-3.73 (m, 1H), 5.45 (br d, J=8.3 Hz, 1H), 7.11-7.15 (m, 2H) 7.06 (d, J=7.3 Hz, 1H), 7.18-7.23 (m, 1H), 12.55-12.68 (m, 1H); ESI-MS m/z [M+H]⁺ 315.4.

### Example 22: 1-(4-fluorobenzyl)-N-((3S,4S)-3-fluoropiperidin-4-yl)cyclopropane-1-carboxamide

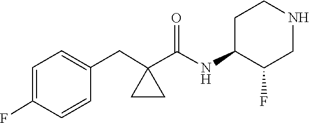 [SMILES: O=C(N[C@H]1CCNC[C@@H]1F)C1(Cc2ccc(F)cc2)CC1]

### Step A: tert-butyl (3S,4S)-3-fluoro-4-(1-(4-fluorobenzyl)cyclopropane-1-carboxamido)piperidine-1-carboxylate

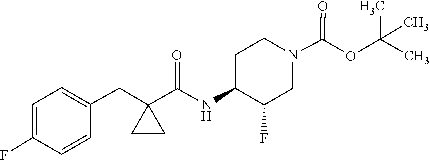 [SMILES: CC(C)(C)OC(=O)N1CC[C@H](NC(=O)C2(Cc3ccc(F)cc3)CC2)[C@@H](F)C1]

A solution of 1-(4-fluorobenzyl)cyclopropane-1-carboxylic acid (100 mg, 0.515 mmol), tert-butyl (3S,4S)-4-amino-3-fluoropiperidine-1-carboxylate (135 mg, 0.618 mmol), HATU (242 mg, 0.618 mmol) and Et₃N (287 μL, 2.06 mmol) in THF (2.575 mL) was stirred at room temperature overnight. The solvent was removed, and the crude material mixed with silica and purified by automated flash silica column chromatography ((ISCO, 4 μg RediSep Rf Gold® column, dry loading) using 50% EtOAc in heptanes to give the title compound as a light yellow oil (0.203 g, quantitative). ESI-MS m/z [M+H]⁺ 395.4.

### Step B: 1-(4-fluorobenzyl)-N-((3S,4S)-3-fluoropiperidin-4-yl)cyclopropane-1-carboxamide

A TFA salt of the title compound was prepared like STEP B of EXAMPLE 15, using tert-butyl (3S,4S)-3-fluoro-4-(1-(4-fluorobenzyl)cyclopropane-1-carboxamido)piperidine-1-carboxylate (0.203 g, 0.515 mmol), and was obtained as a white solid (145 mg, 69%). ¹H NMR (400 MHz, CDCl₃) δ ppm 0.83-0.87 (m, 2H), 1.32-1.36 (m, 2H), 1.47-1.59 (m, 1H), 2.14-2.23 (m, 1H), 2.90-3.09 (m, 6H), 3.29 (ddd, J=16.1, 13.0, 3.5 Hz, 1H), 4.11-4.23 (m, 1H), 4.46-4.65 (m, 1H), 5.61 (br d, J=7.0 Hz, 1H), 7.00-7.07 (m, 2H), 7.28-7.34 (m, 2H); ESI-MS m/z [M+H]⁺ 295.3.

### Example 23: 1-(4-chlorobenzyl)-N-((3S,4S)-3-fluoropiperidin-4-yl)cyclopropane-1-carboxamide

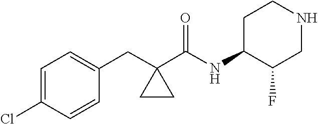 [SMILES: O=C(N[C@H]1CCNC[C@@H]1F)C1(Cc2ccc(Cl)cc2)CC1]

### Step A: tert-butyl (3S,4S)-4-(1-(4-chlorobenzyl)cyclopropane-1-carboxamido)-3-fluoropiperidine-1-carboxylate

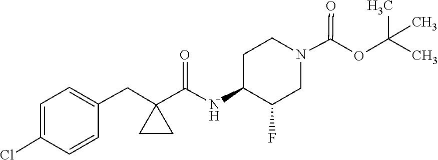 [SMILES: CC(C)(C)OC(=O)N1CC[C@H](NC(=O)C2(Cc3ccc(Cl)cc3)CC2)[C@@H](F)C1]

The title compound was prepared like STEP A of EXAMPLE 22, using 1-(4-chlorobenzyl)cyclopropane-1-carboxylic acid (100 mg, 0.475 mmol), tert-butyl (3S,4S)-4-amino-3-fluoropiperidine-1-carboxylate (124 mg, 0.570 mmol), HATU (223 mg, 0.570 mmol) and Et₃N (265 μL, 1.90 mmol) in THF (2.37 mL), and was obtained as a light yellow semi-solid (170 mg, 87%). ESI-MS m/z [M+H]⁺ 411.4.

### Step B: 1-(4-chlorobenzyl)-N-((3S,4S)-3-fluoropiperidin-4-yl)cyclopropane-1-carboxamide

A TFA salt of the title compound was prepared like STEP B of EXAMPLE 15, using tert-butyl (3S,4S)-4-(1-(4-chlorobenzyl)cyclopropane-1-carboxamido)-3-fluoropiperidine-1-carboxylate (170 mg, 0.414 mmol), and was obtained as a pale white solid (98 mg, 56%). ¹H NMR (400 MHz, CDCl₃) δ ppm 0.82-0.87 (m, 2H), 1.31-1.35 (m, 2H), 1.50-1.61 (m, 1H), 2.14-2.23 (m, 1H), 2.92-3.05 (m, 4H), 3.05-3.13 (m, 1H), 3.24-3.41 (m, 1H), 4.11-4.24 (m, 1H), 4.46-4.68 (m, 1H), 5.65 (br d, J=7.3 Hz, 1H), 7.24-7.32 (m, 4H); ESI-MS m/z [M+H]⁺ 311.3.

### Example 24: N-((3S,4S)-3-fluoropiperidin-4-yl)-1-(2-methylbenzyl)cyclopropane-1-carboxamide

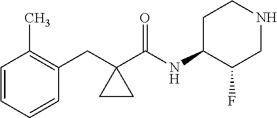 [SMILES: Cc1ccccc1CC1(C(=O)N[C@H]2CCNC[C@@H]2F)CC1]

### Step A: tert-butyl (3S,4S)-3-fluoro-4-(1-(2-methylbenzyl)cyclopropane-1-carboxamido)piperidine-1-carboxylate

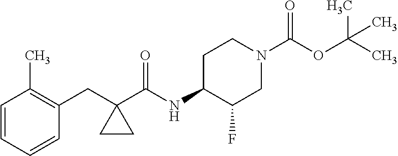 [SMILES: Cc1ccccc1CC1(C(=O)N[C@H]2CCN(C(=O)OC(C)(C)C)C[C@@H]2F)CC1]

The title compound was prepared like STEP A of EXAMPLE 22, using 1-(2-methylbenzyl)cyclopropane-1-carboxylic acid (100 mg, 0.526 mmol), tert-butyl (3S,4S)-4-amino-3-fluoropiperidine-1-carboxylate (138 mg, 0.631 mmol), HATU (245 mg, 0.631 mmol) and Et₃N (293 μL, 2.10 mmol) in THF (2.63 mL), and was obtained as a pale yellow solid (191 mg, 93%). ESI-MS m/z [M+H]⁺ 391.4.

### Step B: N-((3S,4S)-3-fluoropiperidin-4-yl)-1-(2-methylbenzyl)cyclopropane-1-carboxamide

A TFA salt of the title compound was prepared like STEP B of EXAMPLE 15, using tert-butyl (3S,4S)-3-fluoro-4-(1-(2-methylbenzyl)cyclopropane-1-carboxamido)piperidine-1-carboxylate (191 mg, 0.489 mmol), and was obtained as a white solid (145 mg, 73%). ¹H NMR (400 MHz, CDCl₃) δ ppm 0.79-0.85 (m, 2H), 1.37-1.42 (m, 2H), 1.46-1.56 (m, 1H), 2.15-2.25 (m, 2H), 2.30-2.33 (m, 3H), 2.79-2.89 (m, 1H), 2.90-3.00 (m, 3H), 3.00-3.18 (m, 2H), 4.11-4.24 (m, 1H), 4.44-4.67 (m, 1H), 5.56 (d, J=7.3 Hz, 1H), 7.16-7.24 (m, 3H), 7.35-7.40 (m, 1H); ESI-MS m/z [M+H]⁺ 291.4.

### Example 25: 2,2-difluoro-N-((3S,4S)-3-methylpiperidin-4-yl)-2-phenoxyacetamide

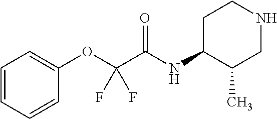 [SMILES: C[C@H]1CNCC[C@@H]1NC(=O)C(F)(F)Oc1ccccc1]

### Step A: tert-butyl (3S,4S)-4-(2,2-difluoro-2-phenoxyacetamido)-3-methylpiperidine-1-carboxylate

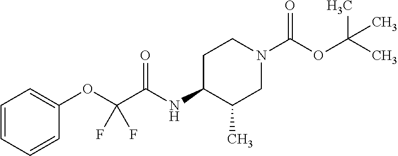 [SMILES: C[C@H]1CN(C(=O)OC(C)(C)C)CC[C@@H]1NC(=O)C(F)(F)Oc1ccccc1]

The title compound was prepared like STEP A of EXAMPLE 15, using 2,2-difluoro-2-phenoxyacetic acid (47 mg, 0.250 mmol), tert-butyl (3S,4S)-4-amino-3-methylpiperidine-1-carboxylate (64.2 mg, 0.300 mmol), HATU (116 mg, 0.300 mmol) and Et₃N (139 μL, 0.999 mmol) in DMA (1.25 mL), and was obtained as a white solid (41 mg, 43%). ESI-MS m/z [M-t-Bu]⁺ 329.3.

### Step B: 2,2-difluoro-N-((3S,4S)-3-methylpiperidin-4-yl)-2-phenoxyacetamide

A TFA salt of the title compound was prepared like STEP B of EXAMPLE 15, using tert-butyl (3S,4S)-4-(2,2-difluoro-2-phenoxyacetamido)-3-methylpiperidine-1-carboxylate (41 mg, 0.107 mmol) and HCl in dioxane (4 M, 160 μL, 0.640 mmol), DCM (213 μL) and MeOH (0.5 mL), and was obtained as a white solid (35 mg, 82%). ¹H NMR (400 MHz, CDCl₃) δ ppm 0.96-1.05 (m, 3H), 1.85-2.02 (m, 1H), 2.02-2.16 (m, 3H), 2.64-2.76 (m, 1H), 2.94-3.09 (m, 1H), 3.40-3.50 (m, 1H), 3.52-3.61 (m, 1H), 3.82-3.91 (m, 1H), 6.43-6.50 (m, 1H), 7.24 (br d, J=8.3 Hz, 2H), 7.28-7.32 (m, 1H), 7.36-7.43 (m, 2H); ESI-MS m/z [M+H]⁺ 285.3.

### Example 26: 2,2-difluoro-N-((3S,4S)-3-methylpiperidin-4-yl)-3-phenylpropanamide

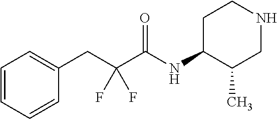 [SMILES: C[C@H]1CNCC[C@@H]1NC(=O)C(F)(F)Cc1ccccc1]

A TFA salt of the title compound was prepared like EXAMPLE 15, using 2,2-difluoro-3-phenylpropanoic acid (50 mg, 0.269 mmol), and was obtained as a white solid (15 mg, 52%). ¹H NMR (400 MHz, CDCl₃) δ ppm 0.70 (d, J=6.3 Hz, 3H), 1.52-1.69 (m, 1H), 1.85-1.92 (m, 1H), 2.48-2.62 (m, 1H), 2.82-3.10 (m, 3H), 3.25-3.49 (m, 4H), 3.58-3.70 (m, 1H), 6.00 (br d, J=8.5 Hz, 1H), 7.24-7.33 (m, 5H), 9.14-9.52 (m, 2H); ESI-MS m/z [M+H]⁺ 283.4.

### Example 27: 1-(3-chlorobenzyl)-N-((3S,4S)-3-fluoropiperidin-4-yl)cyclopropane-1-carboxamide

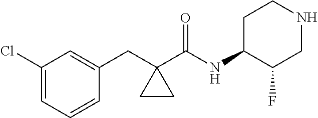 [SMILES: O=C(N[C@H]1CCNC[C@@H]1F)C1(Cc2cccc(Cl)c2)CC1]

### Step A: tert-butyl (3S,4S)-4-(1-(3-chlorobenzyl)cyclopropane-1-carboxamido)-3-fluoropiperidine-1-carboxylate

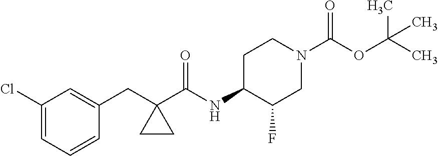 [SMILES: CC(C)(C)OC(=O)N1CC[C@H](NC(=O)C2(Cc3cccc(Cl)c3)CC2)[C@@H](F)C1]

The title compound was prepared like STEP A of EXAMPLE 15, using 1-(3-chlorobenzyl)cyclopropane-1-carboxylic acid (73 mg, 0.347 mmol), tert-butyl (3S,4S)-4-amino-3-fluoropiperidine-1-carboxylate (91 mg, 0.416 mmol), HATU (158 mg, 0.416 mmol) and Et₃N (193 μL, 1.386 mmol) in DMA (1.73 mL), and was obtained as a white solid (74 mg, 52%). ESI-MS m/z [M+H]⁺ 411.5.

### Step B: 1-(3-chlorobenzyl)-N-((3S,4S)-3-fluoropiperidin-4-yl)cyclopropane-1-carboxamide

A TFA salt of the title compound was prepared like STEP B of EXAMPLE 15, using tert-butyl (3S,4S)-4-(1-(3-chlorobenzyl)cyclopropane-1-carboxamido)-3-fluoropiperidine-1-carboxylate (74 mg, 0.180 mmol) and HCl in dioxane (4 M, 270 μL, 1.08 mmol) and DCM (0.6 mL), and was obtained as a white solid (67 mg, 88%). ¹H NMR (400 MHz, CDCl₃) δ ppm 0.85-0.92 (m, 2H), 1.31-1.38 (m, 2H), 1.57-1.69 (m, 1H), 2.12-2.23 (m, 2H), 3.00 (d, J=3.8 Hz, 2H), 3.02-3.16 (m, 3H) 3.33 (br t, J=14.9 Hz, 1H), 4.20 (br d, J=3.8 Hz, 1H), 4.53-4.77 (m, 1H), 5.94 (br d, J=7.3 Hz, 1H), 7.19-7.23 (m, 1H), 7.24-7.31 (m, 2H), 7.33 (s, 1H); ESI-MS m/z [M+H]⁺ 311.3.

### Example 28:2,2-difluoro-N-(1-methylpiperidin-4-yl)-2-phenoxyacetamide

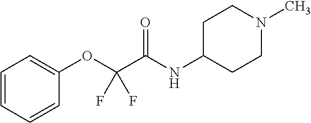 [SMILES: CN1CCC(NC(=O)C(F)(F)Oc2ccccc2)CC1]

A TFA salt of title compound was prepared like STEP A of EXAMPLE 15, using 2,2-difluoro-2-phenoxyacetic acid (61 mg, 0.324 mmol), 1-methylpiperidin-4-amine (55.5 mg, 0.486 mmol), HATU (189 mg, 0.486 mmol) and DIPEA (231 μL, 1.297 mmol) in DMA (1.62 mL), and was obtained as a colorless oil (8 mg, 6%). ¹H NMR (400 MHz, CDCl₃) δ ppm 2.17 (m, 4H), 2.80 (m, 5H), 3.63 (br d, J=12.0 Hz, 2H), 4.10 (br d, J=3.5 Hz, 1H), 7.03 (br d, J=6.8 Hz, 1H), 7.22 (d, J=7.8 Hz, 2H), 7.25-7.30 (m, 1H), 7.35-7.42 (m, 2H); ESI-MS m/z [M+H]⁺ 285.3.

### Example 29: 2,2-difluoro-N-((3S,4S)-3-fluoropiperidin-4-yl)-2-phenoxyacetamide

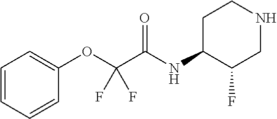 [SMILES: O=C(N[C@H]1CCNC[C@@H]1F)C(F)(F)Oc1ccccc1]

### Step A: tert-butyl (3S,4S)-4-(2,2-difluoro-2-phenoxyacetamido)-3-fluoropiperidine-1-carboxylate

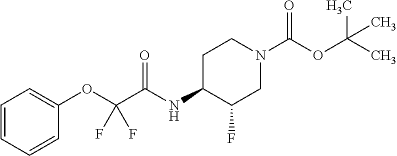 [SMILES: CC(C)(C)OC(=O)N1CC[C@H](NC(=O)C(F)(F)Oc2ccccc2)[C@@H](F)C1]

The title compound was prepared like STEP A of EXAMPLE 15, using 2,2-difluoro-2-phenoxyacetic acid (133 mg, 0.707 mmol), tert-butyl (3S,4S)-4-amino-3-fluoropiperidine-1-carboxylate (185 mg, 0.848 mmol), HATU (329 mg, 0.848 mmol) and DIPEA (494 μL, 2.83 mmol) in DMA (3.54 mL), and was obtained as a colorless oil (275 mg).

### Step B: 2,2-difluoro-N-((3S,4S)-3-fluoropiperidin-4-yl)-2-phenoxyacetamide

A TFA salt of the title compound was prepared like STEP B of EXAMPLE 15, using tert-butyl (3S,4S)-4-(2,2-difluoro-2-phenoxyacetamido)-3-fluoropiperidine-1-carboxylate (275 mg, 0.707 mmol) and HCl in dioxane (4 M, 1.77 mL, 7.07 mmol) and DCM (2.83 mL), and was obtained as a white solid (116 mg, 41% over two steps). ¹H NMR (400 MHz, DMSO-d₆) δ ppm 1.69-1.82 (m, 1H), 1.93-2.06 (m, 1H), 3.02-3.11 (m, 1H), 3.12-3.22 (m, 2H), 3.60 (ddd, J=12.1, 7.6, 4.4 Hz, 1H), 4.13-4.32 (m, 1H), 4.66-4.91 (m, 1H), 7.27 (d, J=7.5 Hz, 2H), 7.31-7.36 (m, 1H), 7.43-7.50 (m, 2H), 9.39 (br d, J=8.5 Hz, 1H); ESI-MS m/z [M+H]⁺ 289.3.

### Example 30: 1-(4-fluoro-2-methylbenzyl)-N-((3S,4S)-3-fluoropiperidin-4-yl)cyclopropane-1-carboxamide

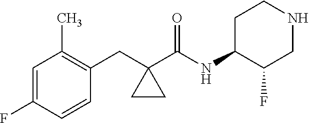 [SMILES: Cc1cc(F)ccc1CC1(C(=O)N[C@H]2CCNC[C@@H]2F)CC1]

### Step A: tert-butyl (3S,4S)-3-fluoro-4-(1-(4-fluoro-2-methylbenzyl)cyclopropane-1-carboxamido)piperidine-1-carboxylate

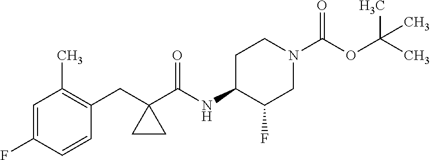 [SMILES: Cc1cc(F)ccc1CC1(C(=O)N[C@H]2CCN(C(=O)OC(C)(C)C)C[C@@H]2F)CC1]

A solution of 1-(4-fluoro-2-methylbenzyl)cyclopropane-1-carboxylic acid (103 mg, 0.495 mmol), tert-butyl (3S,4S)-4-amino-3-fluoropiperidine-1-carboxylate (130 mg, 0.594 mmol), HATU (230 mg, 0.594 mmol) and Et₃N (276 μL, 1.98 mmol) in DMA (2.47 mL) was stirred at room temperature for 48 hours. The solvent was removed under reduced pressure and the residue was purified by automated flash silica column chromatography (ISCO, 40 g RediSep Rf Gold® column, dry loading) using a gradient of 20-100% EtOAc in heptanes. The product-containing fractions were evaporated to give the title compound (202 mg). ESI-MS m/z [M+H]⁺ 409.5.

### Step B: 1-(4-fluoro-2-methylbenzyl)-N-((3S,4S)-3-fluoropiperidin-4-yl)cyclopropane-1-carboxamide

A TFA salt of the title compound was prepared like STEP B of EXAMPLE 15, using tert-butyl (3S,4S)-3-fluoro-4-(1-(4-fluoro-2-methylbenzyl)cyclopropane-1-carboxamido)piperidine-1-carboxylate (202 mg, 0.495 mmol) and HCl in dioxane (4 M, 1.24 mL, 4.95 mmol), DCM (1.24 mL) and MeOH (1.24 mL), and was obtained as a white solid (106 mg, 51% over two steps). ¹H NMR (400 MHz, CDCl₃) δ ppm 0.75-0.80 (m, 2H), 1.33-1.39 (m, 2H), 1.55-1.68 (m, 1H), 2.14-2.25 (m, 1H), 2.30 (s, 3H), 2.93 (s, 2H), 2.96-3.14 (m, 3H), 3.27-3.40 (m, 1H), 4.16-4.28 (m, 1H), 4.51-4.71 (m, 1H), 5.73 (br d, J=7.5 Hz, 1H), 6.85 (td, J=8.4, 2.8 Hz, 1H), 6.91 (dd, J=9.5, 2.5 Hz, 1H), 7.24-7.30 (m, 1H); ESI-MS m/z [M+H]⁺ 309.4.

### Example 31: 1-(4-fluoro-3-methylbenzyl)-N-((3S,4S)-3-fluoropiperidin-4-yl)cyclopropane-1-carboxamide

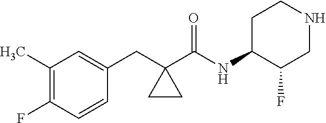 [SMILES: Cc1cc(CC2(C(=O)N[C@H]3CCNC[C@@H]3F)CC2)ccc1F]

### Step A: tert-butyl (3S,4S)-3-fluoro-4-(1-(4-fluoro-3-methylbenzyl)cyclopropane-1-carboxamido)piperidine-1-carboxylate

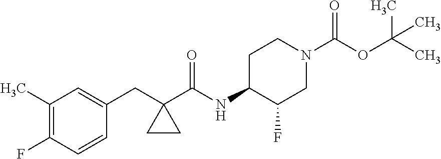 [SMILES: Cc1cc(CC2(C(=O)N[C@H]3CCN(C(=O)OC(C)(C)C)C[C@@H]3F)CC2)ccc1F]

The title compound was prepared like STEP A of EXAMPLE 15, using 1-(4-fluoro-3-methylbenzyl)cyclopropane-1-carboxylic acid (155 mg, 0.744 mmol), tert-butyl (3S,4S)-4-amino-3-fluoropiperidine-1-carboxylate (205 mg, 0.893 mmol), HATU (375 mg, 0.968 mmol) and Et₃N (415 μL, 2.98 mmol) in DMA (3.72 mL), and was obtained as a white solid (258 mg, 83%). ESI-MS m/z [M+H]⁺ 409.5.

### Step B: 1-(4-fluoro-3-methylbenzyl)-N-((3S,4S)-3-fluoropiperidin-4-yl)cyclopropane-1-carboxamide

A TFA salt of the title compound was prepared like STEP B of EXAMPLE 15, using tert-butyl (3S,4S)-3-fluoro-4-(1-(4-fluoro-3-methylbenzyl)cyclopropane-1-carboxamido)piperidine-1-carboxylate (258 mg, 0.632 mmol) and HCl in dioxane (4 M, 1.58 mL, 6.32 mmol) and DCM (1.58 mL), and was obtained as a white solid (207 mg, 78%). ¹H NMR (400 MHz, CDCl₃) δ ppm 0.75-0.91 (m, 2H), 1.25-1.39 (m, 2H), 1.46-1.63 (m, 1H), 2.13-2.21 (m, 1H), 2.25 (d, J=1.8 Hz, 3H), 2.92 (s, 2H), 2.94-3.10 (m, 3H), 3.24-3.40 (m, 1H), 4.17 (td, J=7.7, 4.6 Hz, 1H), 4.44-4.70 (m, 1H), 5.74 (d, J=7.5 Hz, 1H), 6.90-7.01 (m, 1H), 7.06-7.18 (m, 2H); ESI-MS m/z [M+H]⁺ 309.3.

### Example 32: 2-(2-cyanophenoxy)-2,2-difluoro-N-(piperidin-4-yl)acetamide

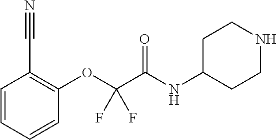 [SMILES: N#Cc1ccccc1OC(F)(F)C(=O)NC1CCNCC1]

### Step A: tert-butyl 4-(2-(2-cyanophenoxy)-2,2-difluoroacetamido)piperidine-1-carboxylate

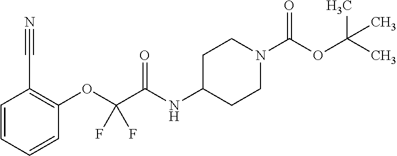 [SMILES: CC(C)(C)OC(=O)N1CCC(NC(=O)C(F)(F)Oc2ccccc2C#N)CC1]

The title compound was prepared like STEP A of EXAMPLE 15, using 2-(2-cyanophenoxy)-2,2-difluoroacetic acid (50 mg, 0.235 mmol), tert-butyl 4-aminopiperidine-1-carboxylate (48.4 mg, 0.235 mmol), HATU (91 mg, 0.235 mmol) and Et₃N (131 μL, 0.938 mmol) in DMA (1.17 mL), and was obtained as a colorless oil. ESI-MS m/z [M+Na]⁺ 418.4.

### Step B: 2-(2-cyanophenoxy)-2,2-difluoro-N-(piperidin-4-yl)acetamide

A TFA salt of the title compound was prepared like STEP B of EXAMPLE 15, using tert-butyl 4-(2-(2-cyanophenoxy)-2,2-difluoroacetamido)piperidine-1-carboxylate, and was obtained as a colorless oil (13 mg, 14% over two steps). ¹H NMR (400 MHz, DMSO-d₆) δ ppm 1.65-1.81 (m, 2H), 1.90 (br dd, J=13.0, 2.3 Hz, 2H), 2.93-3.11 (m, 2H), 3.30 (br d, J=12.8 Hz, 2H), 3.95 (tdt, J=11.1, 7.40, 4.0 Hz, 1H), 7.47-7.57 (m, 2H), 7.76-7.86 (m, 1H), 7.98 (dd, J=7.9, 1.6 Hz, 1H), 9.35 (br d, J=7.5 Hz, 1H); ESI-MS m/z [M+H]⁺ 296.3.

### Example 33 (NB011692-080-002): 2-(2-cyanophenoxy)-2,2-difluoro-N-((3S,4S)-3-methylpiperidin-4-yl)acetamide

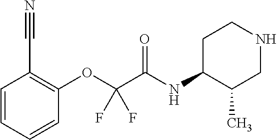 [SMILES: C[C@H]1CNCC[C@@H]1NC(=O)C(F)(F)Oc1ccccc1C#N]

### Step A: tert-butyl (3S,4S)-4-(2-(2-cyanophenoxy)-2,2-difluoroacetamido)-3-methylpiperidine-1-carboxylate

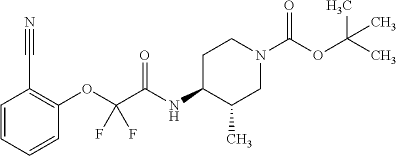 [SMILES: C[C@H]1CN(C(=O)OC(C)(C)C)CC[C@@H]1NC(=O)C(F)(F)Oc1ccccc1C#N]

The title compound was prepared like STEP A of EXAMPLE 15, using 2-(2-cyanophenoxy)-2,2-difluoroacetic acid (99 mg, 0.464 mmol), tert-butyl 4-aminopiperidine-1-carboxylate (119 mg, 0.557 mmol), HATU (216 mg, 0.557 mmol) and Et₃N (259 μL, 1.89 mmol) in DMA (2.32 mL), and was obtained as a colorless oil (23 mg, 12%). ESI-MS [M+Na]⁺ 432.5.

### Step B: 2-(2-cyanophenoxy)-2,2-difluoro-N-((3S,4S)-3-methylpiperidin-4-yl)acetamide

A TFA salt of the title compound was prepared like STEP B of EXAMPLE 15, using tert-butyl (3S,4S)-4-(2-(2-cyanophenoxy)-2,2-difluoroacetamido)-3-methylpiperidine-1-carboxylate (23 mg, 0.056 mmol) and HCl in dioxane (4 M, 140 μL, 0.562 mmol) and DCM (140 μL), and was obtained as a colorless oil (18 mg, 76%). ¹H NMR (400 MHz, CDCl₃) δ ppm 1.10 (br d, J=5.5 Hz, 3H), 1.93-2.05 (m, 1H), 2.21-2.29 (m, 1H), 2.64-2.78 (m, 3H), 2.98-3.11 (m, 1H), 3.40-3.49 (m, 1H), 3.50-3.61 (m, 1H), 3.80-3.94 (m, 1H), 6.94 (br d, J=8.5 Hz, 1H), 7.39-7.45 (m, 1H), 7.50 (d, J=8.0 Hz, 1H), 7.64-7.76 (m, 2H); ESI-MS [M+H]⁺ 310.3.

### Example 34: 2,2-difluoro-N-(piperidin-4-yl)-2-(p-tolyloxy)acetamide

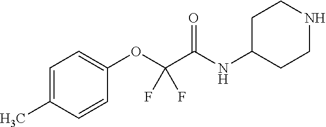 [SMILES: Cc1ccc(OC(F)(F)C(=O)NC2CCNCC2)cc1]

### Step A: tert-butyl 4-(2,2-difluoro-2-(p-tolyloxy)acetamido)piperidine-1-carboxylate

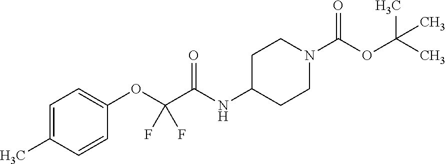 [SMILES: Cc1ccc(OC(F)(F)C(=O)NC2CCN(C(=O)OC(C)(C)C)CC2)cc1]

A solution of 2,2-difluoro-2-(p-tolyloxy)acetic acid (261 mg, 1.29 mmol), tert-butyl 4-aminopiperidine-1-carboxylate (404 mg, 1.94 mmol), HATU (751 mg, 1.94 mmol) and Et₃N (720 μL, 5.16 mmol) in THF (6.46 mL) was stirred at room temperature overnight. The solvent was removed under reduced pressure, and the residue was purified by automated flash silica column chromatography (ISCO, 4 μg RediSep Rf Gold® column, dry loading) using a gradient of 0-100% EtOAc in heptanes, to give the title compound as a pale yellow oil (74 mg, 15%). ESI-MS m/z [M+Na]⁺ 407.5.

### Step B: 2,2-difluoro-N-(piperidin-4-yl)-2-(p-tolyloxy)acetamide

A TFA salt of the title compound was prepared like STEP B of EXAMPLE 15, using tert-butyl 4-(2,2-difluoro-2-(p-tolyloxy)acetamido)piperidine-1-carboxylate (74 mg, 0.192 mmol) and HCl in dioxane (4 M, 481 μL, 1.92 mmol) and MeOH (642 μL), and was obtained as a white solid (46 mg, 60%). ¹H NMR (400 MHz, DMSO-d₆) δ ppm 1.63-1.77 (m, 2H), 1.87 (br dd, J=13.6, 2.3 Hz, 2H), 2.28-2.34 (m, 3H), 2.93-3.06 (m, 2H), 3.29 (br d, J=12.8 Hz, 2H), 3.87-3.98 (m, 1H), 7.13 (d, J=8.5 Hz, 2H), 7.24 (d, J=8.0 Hz, 2H), 8.21-8.36 (m, 1H), 8.49-8.62 (m, 1H); ESI-MS m/z [M+H]⁺ 285.3.

### Example 35: N-((3S,4S)-1,3-dimethylpiperidin-4-yl)-1-(4-fluorobenzyl)cyclopropane-1-carboxamide

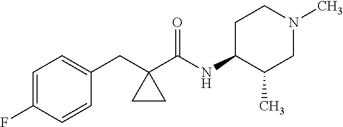 [SMILES: C[C@H]1CN(C)CC[C@@H]1NC(=O)C1(Cc2ccc(F)cc2)CC1]

A solution of 1-(4-fluorobenzyl)-N-((3S,4S)-3-methylpiperidin-4-yl)cyclopropane-1-carboxamide TFA salt (122 mg, 0.302 mmol) in MeOH (2.01 mL) was treated with Et₃N (105 μL, 0.754 mmol) and aqueous formaldehyde (37 wt %, 67.4 μL, 0.905 mmol) at room temperature. The mixture was stirred for 30 minutes. Next, sodium triacetoxyborohydride (198 mg, 0.905 mmol) was added in one portion and stirring was continued at room temperature for 16 hours. The reaction mixture was then diluted with MeOH (1 mL), filtered through a hydrophilic PTFE 0.45 m Millipore® filter and purified by preparative HPLC (Phenomenex Gemini® NX—C18, 5 μm, ID 30 mm×100 mm) using a gradient of 10-100% ACN in water (Acid Mode). The pure fractions were combined and lyophilized to give a TFA salt of the title compound as a white solid (112 mg, 89%). ¹H NMR (400 MHz, CDCl₃) δ ppm 0.64 (d, J=6.8 Hz, 3H), 0.74-0.90 (m, 2H), 1.19-1.28 (m, 1H), 1.32-1.40 (m, 1H), 1.71-1.85 (m, 1H), 1.89-2.01 (m, 2H), 2.31-2.43 (m, 1H), 2.63-2.73 (m, 1H), 2.75 (s, 3H), 2.84 (d, J=16.3 Hz, 1H), 3.05 (d, J=16.3 Hz, 1H), 3.36-3.46 (m, 1H), 3.48-3.56 (m, 1H), 3.58-3.70 (m, 1H), 5.41 (br d, J=8.5 Hz, 1H), 6.97-7.04 (m, 2H), 7.25-7.31 (m, 2H), 13.14-13.26 (m, 1H); ESI-MS m/z [M+H]⁺ 305.5.

### Example 36: 1-(3-chlorobenzyl)-N-((3S,4S)-1,3-dimethylpiperidin-4-yl)cyclopropane-1-carboxamide

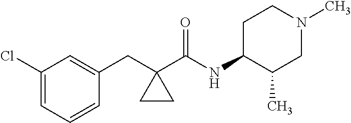 [SMILES: C[C@H]1CN(C)CC[C@@H]1NC(=O)C1(Cc2cccc(Cl)c2)CC1]

A solution of 1-(3-chlorobenzyl)-N-((3S,4S)-3-methylpiperidin-4-yl)cyclopropane-1-carboxamide TFA salt (72 mg, 0.302 mmol) in MeOH (1141 μL) was treated with Et₃N (60 μL, 0.428 mmol) and aqueous formaldehyde (37 wt %, 38.2 μL, 0.513 mmol) at room temperature. The solution was stirred for 30 minutes. Next, Sodium triacetoxyborohydride (112 mg, 0.513 mmol) was added and stirring was continued at room temperature for 16 hours. The reaction mixture was then diluted with MeOH (1 mL), filtered through a hydrophilic PTFE 0.45 m Millipore® filter and purified by preparative HPLC (Phenomenex Gemini® NX—C18, 5 μm, ID 30 mm×100 mm) using a gradient of 10-100% ACN in water (Acid Mode). The pure fractions were combined and lyophilized to give a TFA salt of the title compound as a colorless oil (60 mg, 81%). ¹H NMR (400 MHz, CDCl₃) δ ppm 0.68 (d, J=6.5 Hz, 3H), 0.78-0.90 (m, 2H), 1.20-1.28 (m, 1H), 1.33-1.40 (m, 1H), 1.71-1.85 (m, 1H), 1.89-2.01 (m, 2H), 2.33-2.45 (m, 1H), 2.64-2.73 (m, 1H), 2.77 (s, 3H), 2.85 (d, J=16.6 Hz, 1H), 3.06 (d, J=16.3 Hz, 1H), 3.38-3.46 (m, 1H), 3.50-3.57 (m, 1H), 3.60-3.72 (m, 1H), 5.43-5.52 (m, 1H), 7.15-7.19 (m, 1H), 7.20-7.25 (m, 2H), 7.32 (t, J=1.8 Hz, 1H); ESI-MS m/z [M+H]⁺ 321.4.

### Example 37: 1-(4-chlorobenzyl)-N-((3S,4S)-1,3-dimethylpiperidin-4-yl)cyclopropane-1-carboxamide

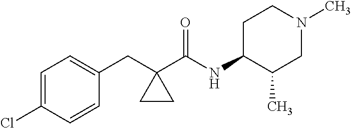 [SMILES: C[C@H]1CN(C)CC[C@@H]1NC(=O)C1(Cc2ccc(Cl)cc2)CC1]

A TFA salt of the title compound was prepared like EXAMPLE 36, using 1-(4-chlorobenzyl)-N-((3S,4S)-3-methylpiperidin-4-yl)cyclopropane-1-carboxamide TFA salt (81 mg, 1.92 mmol), and was obtained as a white solid (71 mg, 85%). ¹H NMR (400 MHz, CDCl₃) δ ppm 0.65 (d, J=6.8 Hz, 3H), 0.76-0.88 (m, 2H), 1.18-1.26 (m, 1H), 1.32-1.39 (m, 1H), 1.75-1.88 (m, 1H), 1.90-2.03 (m, 2H), 2.33-2.44 (m, 1H), 2.64-2.75 (m, 1H), 2.77 (s, 3H), 2.84 (d, J=16.3 Hz, 1H), 3.05 (d, J=16.3 Hz, 1H), 3.39-3.46 (m, 1H), 3.49-3.58 (m, 1H), 3.61-3.71 (m, 1H), 5.48 (br d, J=8.3 Hz, 1H), 7.22-7.30 (m, 4H); ESI-MS m/z [M+H]⁺ 321.4.

### Example 38: N-((3S,4S)-1,3-dimethylpiperidin-4-yl)-1-(2-methylbenzyl)cyclopropane-1-carboxamide

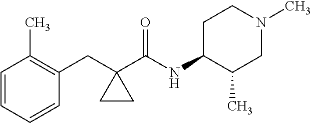 [SMILES: Cc1ccccc1CC1(C(=O)N[C@H]2CCN(C)C[C@@H]2C)CC1]

A TFA salt of the title compound was prepared like EXAMPLE 36, using 1-(2-methylbenzyl)-N-((3S,4S)-3-methylpiperidin-4-yl)cyclopropane-1-carboxamide TFA salt (107 mg, 0.267 mmol), and was obtained as a white solid (81 mg, 73%). ¹H NMR (400 MHz, CDCl₃) δ ppm 0.71 (d, J=6.5 Hz, 3H), 0.74-0.78 (m, 2H), 1.25-1.31 (m, 1H), 1.37-1.42 (m, 1H), 1.65-1.78 (m, 1H), 1.83-1.94 (m, 1H), 1.94-2.01 (m, 1H), 2.31 (s, 3H), 2.34-2.45 (m, 1H), 2.65-2.73 (m, 1H), 2.76 (s, 3H), 2.82 (d, J=16.8 Hz, 1H), 3.07 (d, J=16.8 Hz, 1H), 3.37-3.45 (m, 1H), 3.53 (dt, J=12.0, 1.8 Hz, 1H), 3.62-3.73 (m, 1H), 5.49 (br d, J=8.8 Hz, 1H), 7.10-7.16 (m, 2H), 7.16-7.20 (m, 1H), 7.29-7.35 (m, 1H), 12.71 (br d, J=1.0 Hz, 1H); ESI-MS m/z [M+H]⁺ 301.4.

### Example 39: 1-(4-chlorobenzyl)-N-((3S,4S)-3-fluoro-1-methylpiperidin-4-yl)cyclopropane-1-carboxamide

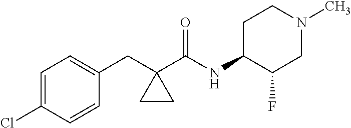 [SMILES: CN1CC[C@H](NC(=O)C2(Cc3ccc(Cl)cc3)CC2)[C@@H](F)C1]

A TFA salt of the title compound was prepared like EXAMPLE 36, using 1-(4-chlorobenzyl)-N-((3S,4S)-3-fluoropiperidin-4-yl)cyclopropane-1-carboxamide TFA salt (16 mg, 0.038 mmol), Et₃N (16 μL, 0.11 mmol), aqueous formaldehyde (37 wt %, 8.4 μL, 0.11 mmol) and sodium triacetoxyborohydride (24.7 mg, 0.113 mmol) in DCM (251 μL), and was obtained as a colorless oil (14 mg, 85%). ¹H NMR (400 MHz, CDCl₃) δ ppm 0.75-0.87 (m, 2H), 1.24-1.35, (m, 2H), 1.73-1.86 (m, 1H), 2.06-2.21 (m, 1H), 2.74-2.92 (m, 5H), 2.93-3.05 (m, 2H), 3.43-3.56 (m, 1H), 3.67-3.82 (m, 1H), 4.10-4.27 (m, 1H), 4.63-4.88 (m, 1H), 5.88-6.02 (m, 1H), 7.19-7.34 (m, 4H); ESI-MS m/z [M+H]⁺ 325.3.

### Example 40: N-((3S,4S)-1,3-dimethylpiperidin-4-yl)-2,2-difluoro-2-phenoxyacetamide

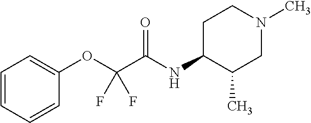 [SMILES: C[C@H]1CN(C)CC[C@@H]1NC(=O)C(F)(F)Oc1ccccc1]

A TFA salt of the title compound was prepared like EXAMPLE 36, using 2,2-difluoro-N-((3S,4S)-3-methylpiperidin-4-yl)-2-phenoxyacetamide TFA salt, and was obtained as a white solid (113 mg, 62%). ¹H NMR (400 MHz, CDCl₃) δ ppm 0.99 (d, J=6.3 Hz, 3H), 2.13-2.19 (m, 3H), 2.29-2.43 (m, 1H), 2.52 (br t, J=12.0 Hz, 1H), 2.82 (s, 3H), 3.53 (br d, J=11.8 Hz, 1H), 3.62 (br d, J=12.6 Hz, 1H), 3.77-3.91 (m, 1H), 6.97 (br d, J=9.0 Hz, 1H), 7.22-7.26 (m, 2H), 7.27-7.31 (m, 1H), 7.35-7.42 (m, 2H); ESI-MS m/z [M+H]⁺ 299.3.

### Example 41: 1-(((6-chloropyridin-2-yl)(methyl)amino)methyl)-N-((2R,4R)-1,2-dimethylpiperidin-4-yl)cyclopropane-1-carboxamide

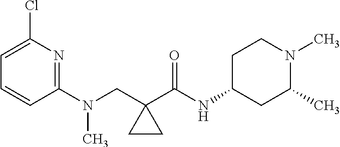 [SMILES: C[C@@H]1C[C@H](NC(=O)C2(CN(C)c3cccc(Cl)n3)CC2)CCN1C]

### Step A: tert-butyl (2R,4R)-4-(1-(((6-chloropyridin-2-yl)(methyl)amino)methyl)cyclopropane-1-carboxamido)-2-methylpiperidine-1-carboxylate

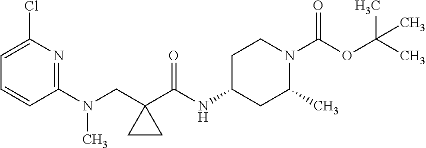 [SMILES: C[C@@H]1C[C@H](NC(=O)C2(CN(C)c3cccc(Cl)n3)CC2)CCN1C(=O)OC(C)(C)C]

To a 40 mL vial were added 1-(((6-chloropyridin-2-yl)(methyl)amino)methyl)cyclopropane-1-carboxylic acid (0.118 g, 0.490 mmol), tert-butyl (2R,4R)-4-amino-2-methylpiperidine-1-carboxylate (0.105 g, 0.490 mmol), HATU (0.186 g, 0.490 mmol), DIPEA (0.256 mL, 1.47 mmol) and DMF (3 mL) to give a yellow solution. The mixture was stirred at room temperature overnight and then diluted with water and extracted with EtOAc. The organic phase was dried over MgSO₄ and concentrated in vacuo to give the title compound (0.214 g). ESI-MS m/z [M+H]⁺ 437.4.

### Step B: 1-(((6-chloropyridin-2-yl)(methyl)amino)methyl)-N-((2R,4R)-2-methylpiperidin-4-yl)cyclopropane-1-carboxamide

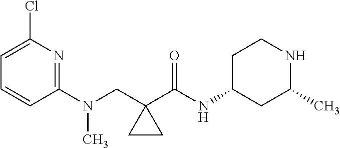 [SMILES: C[C@@H]1C[C@H](NC(=O)C2(CN(C)c3cccc(Cl)n3)CC2)CCN1]

To a 125 mL pear-shaped flask were added tert-butyl (2R,4R)-4-(1-(((6-chloropyridin-2-yl)(methyl)amino)methyl)cyclopropane-1-carboxamido)-2-methylpiperidine-1-carboxylate (0.214 g, 0.49 mmol), HCl in dioxane (4 M, 0.490 mL, 1.96 mmol) and dioxane (3 mL) to give a brown solution. The mixture was stirred at 50° C. for 3 hours and then concentrated in vacuo to give an HCl salt of the title compound as a brown film (0.183 g). ESI-MS m/z [M+H]⁺ 337.3.

### Step C: 1-(((6-chloropyridin-2-yl)(methyl)amino)methyl)-N-((2R,4R)-1,2-dimethylpiperidin-4-yl)cyclopropane-1-carboxamide

In a 125 mL pear-shaped flask, 1-(((6-chloropyridin-2-yl)(methyl)amino)methyl)-N-((2R,4R)-2-methylpiperidin-4-yl)cyclopropane-1-carboxamide HCl (183 mg, 0.49 mmol) and aqueous formaldehyde (37 wt %, 0.076 mL, 0.980 mmol) were dissolved in MeOH (3 mL) to give a brown solution. Next, sodium cyanoborohydride (61.6 mg, 0.980 mmol) was added. The mixture was stirred at room temperature overnight and then purified by preparative HPLC (Phenomenex Gemini® NX—C18, 5 μm, ID 30 mm×100 mm) using a gradient of 10-100% ACN in water (Acid Mode) to give a TFA salt of the title compound (106.4 mg, 47% over three steps). ¹H NMR (400 MHz, CD₃OD) δ ppm 0.87 (d, J=2.8 Hz, 2H), 1.17 (d, J=2.8 Hz, 2H), 1.34 (d, J=6.4 Hz, 3H), 1.38-1.50 (m, 1H), 1.54-1.67 (m, 1H), 1.95-2.04 (m, 2H), 2.84 (s, 3H), 3.01 (s, 3H), 3.03-3.11 (m, 1H), 3.14-3.23 (m, 1H), 3.46-3.53 (m, 1H), 3.85 (s, 3H), 6.57-6.69 (m, 2H), 7.48-7.57 (m, 1H); ESI-MS m/z [M+H]⁺ 351.3.

### Example 42: (R)-2,2-difluoro-2-phenoxy-N-(5-azaspiro[2.4]heptan-7-yl)acetamide

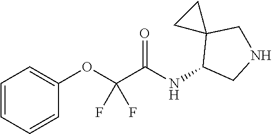 [SMILES: O=C(N[C@H]1CNCC12CC2)C(F)(F)Oc1ccccc1]

A solution of 2,2-difluoro-2-phenoxyacetic acid (93 mg, 0.494 mmol), tert-butyl (R)-7-amino-5-azaspiro[2.4]heptane-5-carboxylate (115 mg, 0.544 mmol), HATU (233 mg, 0.593 mmol) and DIPEA (0.259 mL, 1.483 mmol) in DMF (1.6 mL) was stirred at room temperature overnight. The reaction mixture was then diluted with DCM (20 mL) and washed with 1M HCl and brine. The organic phase was dried over MgSO₄, admixed with TFA (1 mL) and stirred for 4 hours. The mixture was concentrated under reduced pressure and purified by preparative HPLC (Phenomenex Gemini® NX—C18, 5 μm, ID 30 mm×150 mm) using a gradient of 10-100% water/ACN in water (Basic Mode). The pure fractions were combined and lyophilized to give the title compound as a clear oil (21.1 mg, 15%). ¹H NMR (400 MHz, CD₃OD) δ ppm 0.47-0.90 (m, 4H), 2.81-2.95 (m, 1H), 3.08-3.18 (m, 1H), 3.21-3.28 (m, 1H), 3.44-3.59 (m, 1H), 4.02-4.24 (m, 1H), 7.20-7.37 (m, 3H), 7.39-7.50 (m, 2H); ESI-MS m/z [M+H]⁺ 283.3.

### Example 43: 1-(2-fluorobenzyl)-N-((3S,4S)-3-fluoropiperidin-4-yl)cyclopropane-1-carboxamide

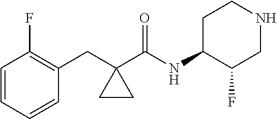 [SMILES: O=C(N[C@H]1CCNC[C@@H]1F)C1(Cc2ccccc2F)CC1]

The title compound was prepared like EXAMPLE 42, using 1-(2-fluorobenzyl)cyclopropane-1-carboxylic acid (100 mg, 0.515 mmol), tert-butyl (3S,4S)-4-amino-3-fluoropiperidine-1-carboxylate (135 mg, 0.618 mmol), HATU (242 mg, 0.618 mmol) and DIPEA (0.27 mL, 1.54 mmol), and was obtained as a clear oil (32.1 mg, 21%). ¹H NMR (400 MHz, CDCl₃) δ ppm 0.85 (d, J=2.4 Hz, 2H), 1.32 (br d, J=2.3 Hz, 3H), 1.90-2.22 (m, 1H), 2.70-2.93 (m, 3H), 3.01 (d, J=2.6 Hz, 2H), 3.12-3.30 (m, 1H), 3.36-4.57 (m, 3H), 5.73 (br s, 1H), 7.03-7.16 (m, 2H), 7.28 (s, 1H), 7.37-7.44 (m, 1H); ESI-MS m/z [M+H]⁺ 295.2.

### Example 44: 1-(3-fluorobenzyl)-N-((3S,4S)-3-fluoropiperidin-4-yl)cyclopropane-1-carboxamide

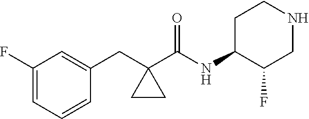 [SMILES: O=C(N[C@H]1CCNC[C@@H]1F)C1(Cc2cccc(F)c2)CC1]

The title compound was prepared like EXAMPLE 42, using 1-(3-fluorobenzyl)cyclopropane-1-carboxylic acid (100 mg, 0.515 mmol), tert-butyl (3S,4S)-4-amino-3-fluoropiperidine-1-carboxylate (135 mg, 0.515 mmol), HATU (242 mg, 0.618 mmol) and DIPEA (0.27 mL, 1.54 mmol), and was obtained as a pale yellow oil (51.9 mg, 34%). ¹H NMR (400 MHz, CDCl₃) δ ppm 0.85 (d, J=2.4 Hz, 2H), 1.32 (br d, J=2.3 Hz, 3H), 1.90-2.22 (m, 1H), 2.70-2.93 (m, 3H), 3.01 (d, J=2.6 Hz, 2H), 3.12-3.30 (m, 1H), 3.36-4.57 (m, 3H), 5.73 (br s, 1H), 7.03-7.16 (m, 2H), 7.28 (s, 1H), 7.37-7.44 (m, 1H); ESI-MS m/z [M+H]⁺ 297.4.

### Example 45: 1-benzyl-N-((3S,4S)-3-fluoropiperidin-4-yl)cyclopropane-1-carboxamide

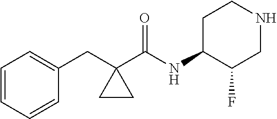 [SMILES: O=C(N[C@H]1CCNC[C@@H]1F)C1(Cc2ccccc2)CC1]

The title compound was prepared like EXAMPLE 42, using 1-benzylcyclopropane-1-carboxylic acid (100 mg, 0.567 mmol), tert-butyl (3S,4S)-4-amino-3-fluoropiperidine-1-carboxylate (136 mg, 0.624 mmol), HATU (267 mg, 0.681 mmol) and DIPEA (0.297 mL, 1.70 mmol), and was obtained as a clear oil (22.1 mg, 14%). ¹H NMR (400 MHz, CDCl₃) δ ppm 0.79-0.87 (m, 2H), 1.04-1.18 (m, 1H), 1.34-1.42 (m, 2H), 1.88-2.01 (m, 1H), 2.60-2.77 (m, 3H), 2.90-3.04 (m, 1H), 3.00 (s, 1H), 3.92-4.05 (m, 1H), 4.10-4.21 (m, 1H), 5.41-5.65 (m, 1H), 7.25-7.30 (m, 1H), 7.37 (s, 4H); ESI-MS m/z [M+H]⁺ 277.3.

### Example 46: N-((3S,4S)-3-fluoropiperidin-4-yl)-1-(3-methoxybenzyl)cyclopropane-1-carboxamide

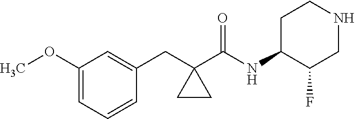 [SMILES: COc1cccc(CC2(C(=O)N[C@H]3CCNC[C@@H]3F)CC2)c1]

The title compound was prepared like EXAMPLE 42, using 1-(3-methoxybenzyl)cyclopropane-1-carboxylic acid (100 mg, 0.485 mmol), tert-butyl (3S,4S)-4-amino-3-fluoropiperidine-1-carboxylate (116 mg, 0.533 mmol), HATU (228 mg, 0.582 mmol) and DIPEA (0.26 mL, 1.45 mmol), and was obtained as a clear oil (21.7 mg, 14.6%). ¹H NMR (400 MHz, CDCl₃) δ ppm 0.85 (d, J=2.6 Hz, 2H), 1.12-1.28 (m, 1H), 1.38 (s, 2H), 1.92-2.08 (m, 1H), 2.68-2.85 (m, 3H), 2.97 (s, 3H), 3.82 (s, 3H), 3.96-4.31 (m, 2H), 3.97-4.20 (m, 2H), 4.21-4.31 (m, 1H), 5.54-5.68 (m, 1H), 6.78-6.86 (m, 1H), 6.90-6.99 (m, 2H), 7.28 (s, 1H); ESI-MS m/z [M+H]⁺ 307.3.

### Example 47: N-((3S,4S)-3-fluoropiperidin-4-yl)-1-(2-methoxybenzyl)cyclopropane-1-carboxamide

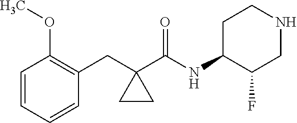 [SMILES: COc1ccccc1CC1(C(=O)N[C@H]2CCNC[C@@H]2F)CC1]

The title compound was prepared like EXAMPLE 42, using 1-(2-methoxybenzyl)cyclopropane-1-carboxylic acid (100 mg, 0.485 mmol), tert-butyl (3S,4S)-4-amino-3-fluoropiperidine-1-carboxylate (106 mg, 0.485 mmol), HATU (228 mg, 0.582 mmol) and DIPEA (0.254 mL, 1.455 mmol), and was obtained as a clear oil (25.3 mg, 17%). ¹H NMR (400 MHz, CDCl₃) δ ppm 0.86 (d, J=2.8 Hz, 2H), 1.35 (s, 3H), 1.99-2.15 (m, 1H), 2.72-2.92 (m, 3H), 2.96 (d, J=7.0 Hz, 2H), 3.09-3.24 (m, 1H), 3.92 (s, 3H), 3.99-4.13 (m, 1H), 4.20-4.49 (m, 1H), 6.26-6.36 (m, 1H), 6.90-7.00 (m, 2H), 7.21-7.28 (m, 1H), 7.34-7.41 (m, 1H); ESI-MS m/z [M+H]⁺ 307.3.

### Example 48: 2,2-difluoro-N-((2S,4S)-2-methylpiperidin-4-yl)-2-phenoxyacetamide

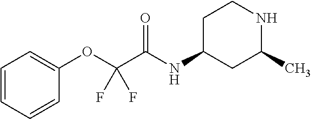 [SMILES: C[C@H]1C[C@@H](NC(=O)C(F)(F)Oc2ccccc2)CCN1]

The title compound was prepared like EXAMPLE 42 using 2,2-difluoro-2-phenoxyacetic acid (93 mg, 0.494 mmol), tert-butyl (2S,4S)-4-amino-2-methylpiperidine-1-carboxylate (117 mg, 0.544 mmol), HATU (233 mg, 0.593 mmol) and DIPEA (0.26 mL, 1.48 mmol), and was obtained as a clear film (8.8 mg, 6%). ¹H NMR (400 MHz, CDCl₃) δ ppm 1.01-1.10 (m, 1H), 1.12-1.16 (m, 3H), 1.28-1.43 (m, 1H), 1.95-2.09 (m, 2H), 2.78 (br d, J=2.5 Hz, 2H), 3.05-3.26 (m, 1H), 3.72-4.06 (m, 1H), 6.19-6.33 (m, 1H), 7.21-7.26 (m, 2H), 7.27-7.31 (m, 1H), 7.35-7.45 (m, 2H); ESI-MS m/z [M+H]⁺ 285.4.

### Example 49: 2,2-difluoro-2-phenoxy-N-(5-azaspiro[2.5]octan-8-yl)acetamide

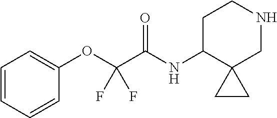 [SMILES: O=C(NC1CCNCC12CC2)C(F)(F)Oc1ccccc1]

The title compound was prepared like EXAMPLE 42 using 2,2-difluoro-2-phenoxyacetic acid (93 mg, 0.494 mmol), tert-butyl 8-amino-5-azaspiro[2.5]octane-5-carboxylate (123 mg, 0.544 mmol), HATU (233 mg, 0.593 mmol) and DIPEA (0.26 mL, 1.48 mmol), and was obtained as a clear oil (8.8 mg, 6%). ¹H NMR (400 MHz, CDCl₃) δ ppm 0.30-1.00 (m, 4H), 2.06-2.26 (m, 2H), 2.81-2.96 (m, 1H), 3.01-3.12 (m, 1H), 3.13-3.44 (m, 2H), 3.79-4.11 (m, 1H), 6.25-6.46 (m, 1H), 7.18-7.25 (m, 4H), 7.28-7.43 (m, 1H), 7.28-7.44 (m, 3H), 9.26-10.31 (m, 1H); ESI-MS m/z [M+H]⁺ 297.4.

### Example 50: 2,2-difluoro-N-(cis-2-methylpiperidin-4-yl)-2-phenoxyacetamide

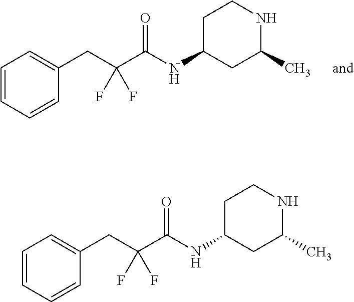 [SMILES: C[C@H]1C[C@@H](NC(=O)C(F)(F)Cc2ccccc2)CCN1 ; C[C@@H]1C[C@H](NC(=O)C(F)(F)Cc2ccccc2)CCN1]

The title compound was prepared like EXAMPLE 42, using 2,2-difluoro-2-phenoxyacetic acid (87 mg, 0.462 mmol), tert-butyl (2S,4S)-4-amino-2-methylpiperidine-1-carboxylate (109 mg, 0.509 mmol), HATU (218 mg, 0.555 mmol) and DIPEA (0.24 mL, 1.39 mmol), and was obtained as a clear film (27.8 mg, 21%). ESI-MS m/z [M+H]⁺ 285.3.

### Example 51: 2,2-difluoro-N-((2R,4R)-2-methylpiperidin-4-yl)-2-phenoxyacetamide

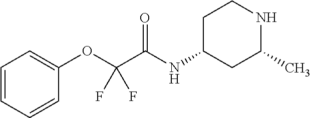 [SMILES: C[C@@H]1C[C@H](NC(=O)C(F)(F)Oc2ccccc2)CCN1]

The title compound was prepared like EXAMPLE 42, using 2,2-difluoro-2-phenoxyacetic acid (87 mg, 0.462 mmol), tert-butyl (2R,4R)-4-amino-2-methylpiperidine-1-carboxylate (109 mg, 0.509 mmol), HATU (218 mg, 0.555 mmol) and DIPEA (0.24 mL, 1.39 mmol), and was obtained as a clear oil (18.6 mg, 14%). ESI-MS m/z [M+H]⁺ 285.3.

### Example 52: 2,2-difluoro-N-((2R,4S)-2-methylpiperidin-4-yl)-2-phenoxyacetamide

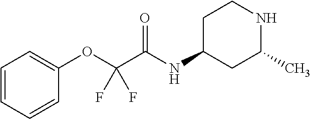 [SMILES: C[C@@H]1C[C@@H](NC(=O)C(F)(F)Oc2ccccc2)CCN1]

The title compound was prepared like EXAMPLE 42, using 2,2-difluoro-2-phenoxyacetic acid (93 mg, 0.494 mmol), tert-butyl (2R,4S)-4-amino-2-methylpiperidine-1-carboxylate (117 mg, 0.544 mmol), HATU (233 mg, 0.593 mmol) and DIPEA (0.26 mL, 1.48 mmol), and was obtained as a clear film (14.3 mg, 10%). ESI-MS m/z [M+H]⁺ 285.3.

### Example 53: 2,2-difluoro-N-((2S,4R)-2-methylpiperidin-4-yl)-2-phenoxyacetamide

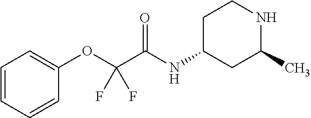 [SMILES: C[C@H]1C[C@H](NC(=O)C(F)(F)Oc2ccccc2)CCN1]

The title compound was prepared like EXAMPLE 42, using 2,2-difluoro-2-phenoxyacetic acid (93 mg, 0.494 mmol), tert-butyl (2S,4R)-4-amino-2-methylpiperidine-1-carboxylate hydrochloride (136 mg, 0.544 mmol), HATU (233 mg, 0.593 mmol) and DIPEA (0.34 mL, 1.98 mmol), and was obtained as a clear film (47 mg, 33%). ESI-MS m/z [M+H]⁺ 285.3.

### Example 54: 2,2-difluoro-2-(2-fluorophenoxy)-N-((3S,4S)-3-methylpiperidin-4-yl)acetamide

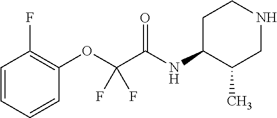 [SMILES: C[C@H]1CNCC[C@@H]1NC(=O)C(F)(F)Oc1ccccc1F]

The title compound was prepared like EXAMPLE 42 using 2,2-difluoro-2-(2-fluorophenoxy)acetic acid (93 mg, 0.451 mmol), tert-butyl (3S,4S)-4-amino-3-methylpiperidine-1-carboxylate (97 mg, 0.451 mmol), HATU (212 mg, 0.541 mmol) and DIPEA (0.394 mL, 2.25 mmol), and was obtained as a clear oil (10 mg, 7.3%). ¹H NMR (400 MHz, CD₃OD) δ ppm 0.96 (m, 3H), 1.61-1.95 (m, 3H), 2.48-2.58 (m, 1H), 2.80-2.91 (m, 1H), 3.15-3.30 (m, 2H), 3.60-3.71 (m, 1H), 7.18-7.44 (m, 4H).

### Example 55: 2,2-difluoro-2-(3-fluorophenoxy)-N-((3S,4S)-3-methylpiperidin-4-yl)acetamide

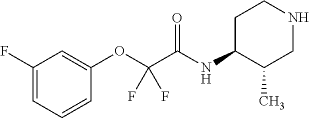 [SMILES: C[C@H]1CNCC[C@@H]1NC(=O)C(F)(F)Oc1cccc(F)c1]

The title compound was prepared like EXAMPLE 42 using 2,2-difluoro-2-(3-fluorophenoxy)acetic acid (82 mg, 0.398 mmol), tert-butyl (3S,4S)-4-amino-3-methylpiperidine-1-carboxylate (85 mg, 0.398 mmol), HATU (187 mg, 0.477 mmol) and DIPEA (0.35 mL, 1.99 mmol), and was obtained as a clear oil (21 mg, 17.4%). ¹H NMR (400 MHz, CD₃OD) δ ppm 0.92 (d, J=6.6 Hz, 3H), 1.57-1.74 (m, 1H), 1.76-2.01 (m, 2H), 2.39-2.58 (m, 1H), 2.77-2.92 (m, 1H), 3.13-3.29 (m, 2H), 3.56-3.72 (m, 1H), 7.04-7.23 (m, 3H), 7.45-7.58 (m, 1H); ESI-MS m/z [M+H]⁺ 303.3.

### Example 56: 2,2-difluoro-N-((3S,4S)-3-methylpiperidin-4-yl)-2-(o-tolyloxy)acetamide

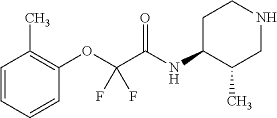 [SMILES: Cc1ccccc1OC(F)(F)C(=O)N[C@H]1CCNC[C@@H]1C]

The title compound was prepared like EXAMPLE 42, using 2,2-difluoro-2-(o-tolyloxy)acetic acid (93 mg, 0.460 mmol), tert-butyl (3S,4S)-4-amino-3-methylpiperidine-1-carboxylate (128 mg, 0.598 mmol), HATU (216 mg, 0.552 mmol) and DIPEA (0.24 mL, 1.38 mmol), and was obtained as a clear oil (19.9 mg, 14%). ¹H NMR (400 MHz, CD₃OD) δ ppm 0.97 (d, J=6.6 Hz, 3H), 1.75-1.93 (m, 1H), 2.00-2.15 (m, 2H), 2.83 (s, 1H), 3.03-3.20 (m, 1H), 3.39-3.53 (m, 2H), 3.68-3.87 (m, 1H), 7.07-7.37 (m, 4H); ESI-MS m/z [M+H]⁺ 299.3.

### Example 57: 2,2-difluoro-N-((3S,4S)-3-methylpiperidin-4-yl)-2-(m-tolyloxy)acetamide

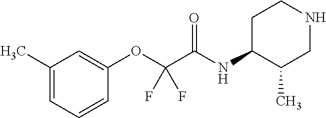 [SMILES: Cc1cccc(OC(F)(F)C(=O)N[C@H]2CCNC[C@@H]2C)c1]

The title compound was prepared like EXAMPLE 42, using 2,2-difluoro-2-(m-tolyloxy)acetic acid (93 mg, 0.460 mmol), tert-butyl (3S,4S)-4-amino-3-methylpiperidine-1-carboxylate (128 mg, 0.598 mmol), HATU (216 mg, 0.552 mmol) and DIPEA (0.24 mL, 1.38 mmol), and was obtained as a clear oil (25.5 mg, 19%). ¹H NMR (400 MHz, CD₃OD) δ ppm 0.94 (d, J=6.6 Hz, 3H), 1.71-1.91 (m, 1H), 1.95-2.10 (m, 2H), 2.36 (d, J=10.3 Hz, 4H), 2.70-2.93 (m, 1H), 3.01-3.20 (m, 1H), 3.36-3.53 (m, 2H), 3.63-3.83 (m, 1H), 6.98-7.33 (m, 2H), 7.13 (br d, J=0.8 Hz, 1H), 7.21 (d, J=7.8 Hz, 1H), 7.26-7.32 (m, 1H); ESI-MS m/z [M+H]⁺ 299.3.

### Example 58: 2,2-difluoro-2-(4-fluorophenoxy)-N-((3S,4S)-3-methylpiperidin-4-yl)acetamide

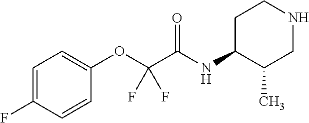 [SMILES: C[C@H]1CNCC[C@@H]1NC(=O)C(F)(F)Oc1ccc(F)cc1]

A solution of 2,2-difluoro-2-(4-fluorophenoxy)acetic acid (87 mg, 0.422 mmol), tert-butyl (3S,4S)-4-amino-3-methylpiperidine-1-carboxylate (90 mg, 0.422 mmol), HATU (199 mg, 0.507 mmol) and DIPEA (369 μL, 2.110 mmol) in DMF (1.41 mL) was stirred at room temperature overnight. The reaction mixture was diluted with DCM. The organic layer was washed with 1 N HCl and brine and dried over Na₂SO₄. Trifluoroacetic acid (1 mL) was added to the organic layer. The mixture was stirred for 4 hours and then purified by preparative HPLC (Phenomenex Gemini® NX—C18, 5 μm, ID 30 mm×150 mm), using a gradient of 10-100% water/ACN in water (Basic Mode). The product was re-purified by preparative HPLC (Phenomenex Gemini® NX—C18, 5 μm, ID 30 mm×100 mm) using a gradient of 10-100% ACN in water (Acid Mode) to give a TFA salt of the title compound as a clear oil. (5.3 mg, 3.0%). ¹H NMR (400 MHz, CD₃OD) δ ppm 0.96 (d, J=6.6 Hz, 3H), 1.77-1.96 (m, 1H), 1.99-2.13 (m, 2H), 2.75-2.89 (m, 1H), 3.04-3.21 (m, 1H), 3.36-3.53 (m, 2H), 3.70-3.82 (m, 1H), 7.08-7.22 (m, 2H), 7.24-7.34 (m, 2H); ESI-MS m/z [M+H]⁺ 303.3.

### Example 59: 1-(3-chlorophenoxy)-N-((3S,4S)-3-fluoropiperidin-4-yl)cyclopropane-1-carboxamide

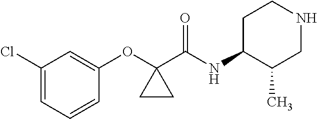 [SMILES: C[C@H]1CNCC[C@@H]1NC(=O)C1(Oc2cccc(Cl)c2)CC1]

In a 4 mL vial were dissolved 1-(3-chlorophenoxy)cyclopropane-1-carboxylic acid (53.2 mg, 0.250 mmol), tert-butyl (3S,4S)-4-amino-3-fluoropiperidine-1-carboxylate (54.6 mg, 0.250 mmol), HATU (95 mg, 0.250 mmol) and DIPEA (87 μL, 0.500 mmol) in DMF (2 mL). The resulting yellow solution was stirred at room temperature overnight. The reaction mixture was diluted with DCM. The organic layer was washed with 1 N HCl and brine and dried over Na₂SO₄. Trifluoroacetic acid (1 mL, 12.98 mmol) was added to the organic layer. The mixture was stirred overnight and then purified by preparative HPLC (Phenomenex Gemini® NX—C18, 5 μm, ID 30 mm×100 mm) using a gradient of 10-100% ACN in water (Acid Mode) to give a TFA salt of the title compound as an off-white solid (59 mg, 55%). ¹H NMR (400 MHz, DMSO-d₆) δ ppm 1.08-1.17 (m, 2H), 1.39-1.47 (m, 2H), 1.58-1.68 (m, 1H), 1.81-1.89 (m, 1H), 2.95-3.03 (m, 1H), 3.06-3.21 (m, 2H), 3.42-3.51 (m, 1H), 4.16-4.21 (m, 1H), 4.58-4.77 (m, 1H), 6.84-6.95 (m, 2H), 7.01-7.11 (m, 1H), 7.33 (t, J=8.2 Hz, 1H) 8.41 (d, J=8.3 Hz, 1H), 8.79-9.06 (m, 1H), 9.20 (br s, 1H); ESI-MS m/z [M+H]⁺ 313.2.

### Example 60: 1-(2-cyclopropylphenoxy)-N-((3S,4S)-3-fluoropiperidin-4-yl)cyclopropane-1-carboxamide

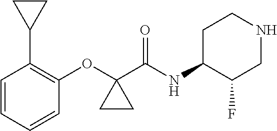 [SMILES: O=C(N[C@H]1CCNC[C@@H]1F)C1(Oc2ccccc2C2CC2)CC1]

To a 4 mL vial were added 1-(2-cyclopropylphenoxy)cyclopropane-1-carboxylic acid (54.6 mg, 0.250 mmol), tert-butyl (3S,4S)-4-amino-3-fluoropiperidine-1-carboxylate (54.6 mg, 0.250 mmol), HATU (95 mg, 0.250 mmol), DIPEA (87 μL, 0.500 mmol) and DMF (2 mL). The resulting yellow solution was stirred at room temperature overnight. The reaction mixture was diluted with DCM. The organic layer was washed with 1 N HCl and brine and dried over Na₂SO₄. Trifluoroacetic acid (1 mL, 12.98 mmol) was added to the organic layer. The mixture was stirred overnight and then purified by preparative HPLC (Phenomenex Gemini® NX—C18, 5 μm, ID 30 mm×100 mm) using a gradient of 10-100% ACN in water (Acid Mode) to give a TFA salt of the title compound as an off-white solid (70 mg, 65%). ¹H NMR (400 MHz, DMSO-d₆) δ ppm 0.56-0.64 (m, 2H), 0.85-0.92 (m, 2H), 1.00-1.10 (m, 2H), 1.35-1.46 (m, 2H), 1.66-1.78 (m, 1H), 1.88-1.96 (m, 1H), 2.07-2.16 (m, 1H), 2.98-3.07 (m, 1H), 3.08-3.17 (m, 1H), 3.18-3.25 (m, 1H), 3.51-3.54 (m, 1H), 4.17-4.29 (m, 1H), 4.67-4.88 (m, 1H), 6.79-6.87 (m, 2H), 6.88-6.95 (m, 1H), 7.04-7.13 (m, 1H), 8.39 (d, J=8.3 Hz, 1H), 8.70-8.97 (m, 1H), 9.12 (br s, 1H); ESI-MS m/z [M+H]⁺ 319.5.

### Example 61: 2-(2,3-dichlorophenoxy)-N-((3S,4S)-3-fluoropiperidin-4-yl)propanamide

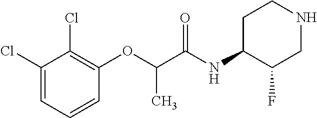 [SMILES: CC(Oc1cccc(Cl)c1Cl)C(=O)N[C@H]1CCNC[C@@H]1F]

To a 4 ml vial were added 2-(2,3-dichlorophenoxy)propanoic acid (58.8 mg, 0.250 mmol), tert-butyl (3S,4S)-4-amino-3-fluoropiperidine-1-carboxylate (54.6 mg, 0.250 mmol), HATU (95 mg, 0.250 mmol), DIPEA (87 μL, 0.500 mmol) and DMF (2 mL). The resulting yellow solution was stirred at room temperature overnight. The reaction mixture was diluted with DCM. The organic layer was washed with 1 N HCl and brine and dried over Na₂SO₄. Trifluoroacetic acid (1 mL, 12.98 mmol) was added to the organic layer. The mixture was stirred overnight and then purified by preparative HPLC (Phenomenex Gemini® NX—C18, 5 μm, ID 30 mm×100 mm) using a gradient of 10-100% ACN in water (Acid Mode) to give a TFA salt of the title compound as an off-white solid (59 mg, 53%). ¹H NMR (400 MHz, DMSO-d₆) δ ppm 1.47-1.54 (m, 3H), 1.60-1.76 (m, 1H), 1.93-2.03 (m, 1H), 3.01-3.10 (m, 1H), 3.16-3.27 (m, 2H), 3.47-3.54 (m, 1H), 4.07-4.19 (m, 1H), 4.60-4.85 (m, 2H), 6.91-6.97 (m, 1H), 7.20-7.31 (m, 2H), 8.52 (br d, J=7.4 Hz, 1H), 9.14 (br s, 2H); ESI-MS m/z [M+H]⁺ 335.2.

### Example 62: 2-(2-chlorophenoxy)-N-((3S,4S)-3-fluoropiperidin-4-yl)propanamide

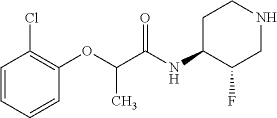 [SMILES: CC(Oc1ccccc1Cl)C(=O)N[C@H]1CCNC[C@@H]1F]

To a 4 mL vial were added 2-(2-chlorophenoxy)propanoic acid (50.2 mg, 0.250 mmol), tert-butyl (3S,4S)-4-amino-3-fluoropiperidine-1-carboxylate (54.6 mg, 0.250 mmol), HATU (95 mg, 0.250 mmol), DIPEA (87 μL, 0.500 mmol) and DMF (2 mL). The resulting yellow solution was stirred at room temperature overnight. The reaction mixture was diluted with DCM. The organic layer was washed with 1 N HCl and brine and dried over Na₂SO₄. Trifluoroacetic acid (1 mL, 12.98 mmol) was added to the organic layer. The mixture was stirred overnight and then purified by preparative HPLC (Phenomenex Gemini® NX—C18, 5 μm, ID 30 mm×100 mm) using a gradient of 10-100% ACN in water (Acid Mode) to give a TFA salt of the title compound as an off-white semisolid (68 mg, 65%). ¹H NMR (400 MHz, CD₃OD) δ ppm 1.67 (dd, J=6.6, 1.2 Hz, 3H), 1.87-2.04 (m, 1H), 2.22-2.37 (m, 1H), 3.20-3.37 (m, 2H), 3.49-3.68 (m, 1H), 4.24-4.37 (m, 1H), 4.79-4.86 (m, 2H), 4.90-5.01 (m, 1H), 7.04-7.13 (m, 2H), 7.24-7.39 (m, 1H), 7.43-7.53 (m, 1H); ESI-MS m/z [M+H]⁺ 301.2.

### Example 63: 2-(2-cyclopropylphenoxy)-N-((3S,4S)-3-fluoropiperidin-4-yl)propanamide

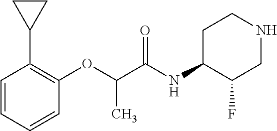 [SMILES: CC(Oc1ccccc1C1CC1)C(=O)N[C@H]1CCNC[C@@H]1F]

To a 4 ml vial were added 2-(2-cyclopropylphenoxy)propanoic acid (51.6 mg, 0.25 mmol), tert-butyl (3S,4S)-4-amino-3-fluoropiperidine-1-carboxylate (54.6 mg, 0.250 mmol), HATU (95 mg, 0.250 mmol), DIPEA (87 μL, 0.500 mmol) and DMF (2 mL). The resulting yellow solution was stirred at room temperature overnight. The reaction mixture was diluted with DCM. The organic layer was washed with 1 N HCl and brine and dried over Na₂SO₄. Trifluoroacetic acid (1 mL, 12.98 mmol) was added to the organic layer. The mixture was stirred overnight and then purified by preparative HPLC (Phenomenex Gemini® NX—C18, 5 μm, ID 30 mm×100 mm) using a gradient of 10-100% ACN in water (Acid Mode) to give a TFA salt of the title compound as an off-white solid (64 mg, 61%). ¹H NMR (400 MHz, CD₃OD) δ ppm 0.64-0.73 (m, 1H), 0.73-0.82 (m, 1H), 0.95-1.07 (m, 2H), 1.65 (dd, J=6.6, 2.1 Hz, 3H), 1.85-2.04 (m, 1H), 2.20-2.39 (m, 2H), 3.20-3.35 (m, 2H), 3.43-3.66 (m, 1H), 4.25-4.35 (m, 1H), 4.73-4.79 (m, 1H), 4.79-4.86 (m, 1H), 4.89-5.01 (m, 1H), 6.87-6.91 (m, 1H), 6.91-6.95 (m, 1H), 6.95-7.01 (m, 1H), 7.09-7.19 (m, 1H); ESI-MS m/z [M+H]⁺ 307.3.

### Example 64: 2-(2-chlorophenoxy)-2,2-difluoro-N-((3S,4S)-3-methylpiperidin-4-yl)acetamide

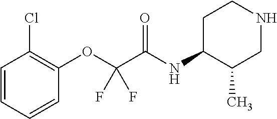 [SMILES: C[C@H]1CNCC[C@@H]1NC(=O)C(F)(F)Oc1ccccc1Cl]

To a solution of 2-(2-chlorophenoxy)-2,2-difluoroacetic acid (100 mg, 0.449 mmol), tert-butyl (3S,4S)-4-amino-3-methylpiperidine-1-carboxylate (96.0 mg, 0.449 mmol) and HATU (205 mg, 0.539 mmol) in DMA (0.899 mL) was added DIPEA (0.172 mL, 0.988 mmol). The reaction mixture was stirred at room temperature for 1 hour and then diluted with water (3 mL). The aqueous layer was decanted off and the oily residue was washed with water (1 mL) and dried under a stream of nitrogen. The resulting oil was treated with 4 M HCl in dioxane (1.12 mL, 4.49 mmol). The mixture was stirred at room temperature for 2 hours and then concentrated in vacuo and purified by preparative HPLC (Phenomenex Gemini® NX—C18, 5 μm, ID 30 mm×150 mm) using a gradient of 20-60% water/ACN in water (Basic Mode) to give the title compound as a white solid (59.0 mg, 41%). ¹H NMR (400 MHz, DMSO-d₆) δ ppm 0.67 (d, J=6.6 Hz, 3H), 1.38 (qd, J=12.2, 4.3 Hz, 1H), 1.49-1.61 (m, 2H), 2.08-2.18 (m, 1H), 2.45-2.49 (m, 1H), 2.83-2.94 (m, 2H), 3.30-3.38 (m, 1H), 7.23-7.31 (m, 1H), 7.32-7.39 (m, 2H), 7.52-7.57 (m, 1H), 8.88 (br d, J=8.8 Hz, 1H); ESI-MS m/z [M+H]⁺ 319.3.

### Example 65: 2-(3-chlorophenoxy)-2,2-difluoro-N-((3S,4S)-3-methylpiperidin-4-yl)acetamide

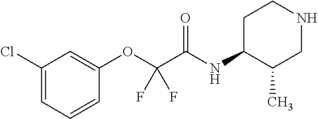 [SMILES: C[C@H]1CNCC[C@@H]1NC(=O)C(F)(F)Oc1cccc(Cl)c1]

To a solution of 2-(3-chlorophenoxy)-2,2-difluoroacetic acid (100 mg, 0.449 mmol), tert-butyl (3S,4S)-4-amino-3-methylpiperidine-1-carboxylate (96 mg, 0.449 mmol) and HATU (205 mg, 0.539 mmol) in DMA (0.899 mL) was added DIPEA (0.313 mL, 1.80 mmol). The reaction mixture was stirred at room temperature for 1 hour and then diluted with water (3 mL). The aqueous later was decanted off and the oily residue was washed with water (1 mL) and dried under a stream of nitrogen. The resulting oil was treated with HCl in dioxane (4 M, 1.12 mL, 4.49 mmol). The mixture was stirred at ambient temperature for 2 hours and then concentrated in vacuo and purified by preparative HPLC (Phenomenex Gemini® NX—C18, 5 μm, ID 30 mm×150 mm) using a gradient of 20-80% water/ACN in water (Basic Mode) to give the title compound as a yellow semisolid (55 mg, 38%). ¹H NMR (400 MHz, DMSO-d₆) δ ppm 0.69 (d, J=6.5 Hz, 3H), 1.40 (qd, J=12.2, 4.3 Hz, 1H), 1.50-1.65 (m, 2H), 2.17 (brt, J=11.6 Hz, 1H), 2.34-2.49 (m, 1H), 2.85-3.02 (m, 2H), 3.37-3.48 (m, 1H), 7.23-7.31 (m, 1H), 7.36-7.44 (m, 2H), 7.46-7.52 (m, 1H), 8.93 (br d, J=8.7 Hz, 1H); ESI-MS m/z [M+H]⁺ 319.3.

### Example 66: 2-(4-chlorophenoxy)-2,2-difluoro-N-((3S,4S)-3-methylpiperidin-4-yl)acetamide

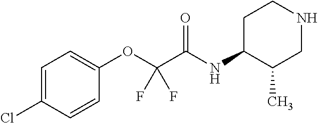 [SMILES: C[C@H]1CNCC[C@@H]1NC(=O)C(F)(F)Oc1ccc(Cl)cc1]

To a solution of 2-(4-chlorophenoxy)-2,2-difluoroacetic acid (100 mg, 0.449 mmol), tert-butyl (3S,4S)-4-amino-3-methylpiperidine-1-carboxylate (96 mg, 0.449 mmol) and HATU (205 mg, 0.539 mmol) in DMA (0.899 mL) was added DIPEA (0.313 mL, 1.80 mmol). The reaction mixture was stirred at room temperature for 1 hour and then diluted with water (3 mL). The aqueous later was decanted off and the oily residue was washed with water (1 mL) and dried under a stream of nitrogen. The resulting oil was treated with HCl in dioxane (4 M, 1.12 mL, 4.49 mmol). The mixture was stirred at ambient temperature for 2 hours and then concentrated in vacuo and purified by preparative HPLC (Phenomenex Gemini® NX—C18, 5 μm, ID 30 mm×150 mm) using a gradient of 20-60% water/ACN in water (Basic Mode) to give the title compound as a yellow semisolid (65 mg, 45%). ¹H NMR (400 MHz, DMSO-d₆) δ ppm 0.61 (d, J=6.5 Hz, 3H), 1.33 (qd, J=12.2, 4.3 Hz, 1H), 1.41-1.59 (m, 2H), 2.09 (t, J=11.7 Hz, 1H), 2.36-2.42 (m, 1H), 2.79-2.92 (m, 2H), 3.28-3.33 (m, 1H), 7.18-7.28 (m, 2H), 7.38-7.51 (m, 2H), 8.84 (br d, J=8.7 Hz, 1H); ESI-MS m/z [M+H]⁺ 319.2.

### Example 67: 2-(2-chlorophenoxy)-N-((2R,4R)-1,2-dimethylpiperidin-4-yl)propanamide

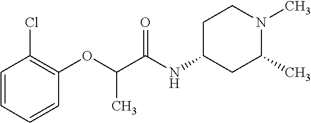 [SMILES: CC(Oc1ccccc1Cl)C(=O)N[C@@H]1CCN(C)[C@H](C)C1]

### Step A: tert-butyl (2R,4R)-4-(2-(2-chlorophenoxy)propanamido)-2-methylpiperidine-1-carboxylate

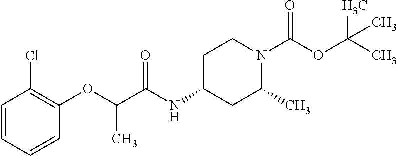 [SMILES: CC(Oc1ccccc1Cl)C(=O)N[C@@H]1CCN(C(=O)OC(C)(C)C)[C@H](C)C1]

To a 4 mL vial were added 2-(2-chlorophenoxy)propanoic acid (0.040 g, 0.200 mmol), tert-butyl (2R,4R)-4-amino-2-methylpiperidine-1-carboxylate (0.043 g, 0.200 mmol), HATU (0.076 g, 0.200 mmol), DIPEA (0.139 mL, 0.800 mmol) and DMF (1.600 mL). The resulting yellow solution was stirred at room temperature overnight. The reaction mixture was diluted with DCM. The organic layer was washed with 1 N HCl and brine, dried over Na₂SO₄ and concentrated in vacuo to give the title compound as an off-white solid (79 mg, 0.200 mmol). ESI-MS m/z [M+H-t-Bu]⁺341.2.

### Step B: 2-(2-chlorophenoxy)-N-((2R,4R)-2-methylpiperidin-4-yl)propanamide

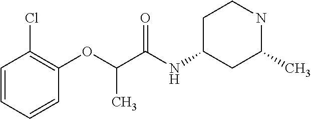 [SMILES: CC(Oc1ccccc1Cl)C(=O)N[C@@H]1CCN[C@H](C)C1]

Crude tert-butyl (2R,4R)-4-(2-(2-chlorophenoxy)propanamido)-2-methylpiperidine-1-carboxylate (0.079 g, 0.200 mmol) and HCl in dioxane (4 M, 0.200 mL, 0.800 mmol) were combined in dioxane (1.227 ml) to give a brown solution. The mixture was stirred at 50° C. for 3 hours. Additional HCl in dioxane (4 M, 0.200 mL, 0.800 mmol) was added and the mixture was stirred at 50° C. for another 3 hours. The mixture was concentrated in vacuo to give the title compound as brown film (59 mg, 0.200 mmol). ESI-MS m/z [M+H]⁺ 297.3.

### Step C: 2-(2-chlorophenoxy)-N-((2R,4R)-1,2-dimethylpiperidin-4-yl)propanamide

In a round-bottomed flask were combined crude 2-(2-chlorophenoxy)-N-((2R,4R)-2-methylpiperidin-4-yl)propanamide (59 mg, 0.200 mmol), formaldehyde (31.2 μL, 0.400 mmol) and MeOH (1.227 mL). To the resulting brown solution was added sodium cyanoborohydride (25.1 mg, 0.400 mmol). The mixture was stirred at room temperature overnight and then purified by preparative HPLC (Phenomenex Gemini® NX—C18, 5 μm, ID 30 mm×150 mm) using a gradient of 10-100% water/ACN in water (Basic Mode). The purification step recovered starting material, which was again reacted under the above conditions and then purified by preparative HPLC (Phenomenex Gemini® NX—C18, 5 μm, ID 30 mm×100 mm) using a gradient of 10-100% ACN in water (Acid Mode) to give a TFA salt of the title compound as a clear oil (26 mg, 31%). ¹H NMR (400 MHz, CD₃GD) δ ppm 1.36-1.74 (m, 8H), 1.77-1.90 (m, 1H), 2.15-2.24 (m, 1H), 2.24-2.34 (m, 1H), 2.95-3.00 (m, 3H), 3.24-3.32 (m, 1H), 3.60-3.66 (m, 1H), 4.09-4.17 (m, 1H), 4.76-4.81 (m, 1H), 7.07-7.12 (m, 2H), 7.31-7.36 (m, 1H), 7.47-7.52 (m, 1H); ESI-MS m/z [M+H]⁺ 311.3.

### Example 68: 1-(3-fluoro-5-methoxybenzyl)-N-((3S,4S)-3-fluoropiperidin-4-yl)cyclopropane-1-carboxamide

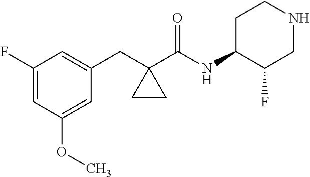 [SMILES: COc1cc(F)cc(CC2(C(=O)N[C@H]3CCNC[C@@H]3F)CC2)c1]

### Step A: tert-butyl (3S,4S)-3-fluoro-4-(1-(3-fluoro-5-methoxybenzyl)cyclopropane-1-carboxamido)piperidine-1-carboxylate

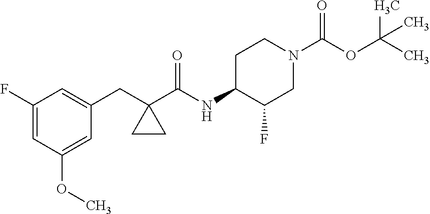 [SMILES: COc1cc(F)cc(CC2(C(=O)N[C@H]3CCN(C(=O)OC(C)(C)C)C[C@@H]3F)CC2)c1]

A solution of 1-(3-fluoro-5-methoxybenzyl)cyclopropane-1-carboxylic acid (200 mg, 0.892 mmol), tert-butyl (3S,4S)-4-amino-3-fluoropiperidine-1-carboxylate (246 mg, 1.07 mmol), HATU (415 mg, 1.07 mmol) and Et₃N (496 μL, 3.57 mmol) in THE (4.46 mL) was stirred at room temperature for 48 hours. The reaction mixture was diluted with MeOH, filtered through a hydrophilic PTFE 0.45 m Millipore® filter and purified by preparative HPLC (Phenomenex Gemini® NX—C18, 5 μm, ID 30 mm×100 mm) using a gradient of 10-100% ACN in water (Acid Mode) to give the title compound as a white solid (300 mg, 79%). ¹H NMR (400 MHz, CDCl₃) δ ppm 0.80-0.85 (m, 2H), 1.09-1.24 (m, 1H), 1.32-1.37 (m, 2H), 1.46 (s, 9H), 1.92-2.04 (m, 1H), 2.82-3.09 (m, 4H), 3.66 (br d, J=13.6 Hz, 1H), 3.86 (s, 3H), 3.93-4.05 (m, 2H), 4.06-4.24 (m, 1H), 6.20 (br s, 1H), 6.79-6.86 (m, 1H), 6.88-6.98 (m, 1H), 7.08 (dd, J=9.0, 3.0 Hz, 1H); ¹⁹F NMR (376 MHz, CDCl₃) δ ppm −75.85 (s, 1F), −122.48 (br s, 1F); ESI-MS m/z [M+H]⁺ 425.5.

### Step B: 1-(3-fluoro-5-methoxybenzyl)-N-((3S,4S)-3-fluoropiperidin-4-yl)cyclopropane-1-carboxamide

A solution of tert-butyl (3S,4S)-3-fluoro-4-(1-(3-fluoro-5-methoxybenzyl)cyclopropane-1-carboxamido)piperidine-1-carboxylate (300 mg, 0.707 mmol) in DCM (2 mL) and MeOH (1 mL) was treated with HCl in dioxane (4 M, 1.77 μL, 7.07 mmol) at room temperature. The reaction mixture was stirred for 16 hours. The solvent was removed under reduced pressure, giving a sticky yellow solid that was treated with ether (3×25 mL). The solvent was removed under reduced pressure, again yielding a sticky solid, so heptanes (25 mL) were added. The solvent was removed under reduced pressure, resulting in a flowing yellow solid. The yellow solid was divided into two batches and purified by automated flash silica column chromatography (ISCO, 4 μg RediSep Rf Gold® column, dry loading) using a gradient of 0-100% EtOAc in heptanes followed by a gradient of 0-30% MeOH in DCM. The pure fractions were concentrated under reduced pressure to give an HCl salt of the title compound as a pale yellow solid (194 mg, 76%). ¹H NMR (400 MHz, DMSO-d₆) δ ppm 0.60-0.67 (m, 2H), 1.00-1.09 (m, 2H), 1.56-1.70 (m, 1H), 1.84 (br dd, J=10.0, 4.8 Hz, 1H), 2.89-3.02 (m, 3H), 3.02-3.12 (m, 1H), 3.14-3.21 (m, 1H), 3.45 (td, J=12.0, 4.1 Hz, 1H), 3.76 (s, 3H), 3.98-4.17 (m, 1H), 4.59-4.84 (m, 1H), 6.89-7.03 (m, 3H), 7.74 (dd, J=4.4, 1.6 Hz, 1H); ESI-MS m/z [M+H]⁺ 325.4.

### Example 69: 2,2-difluoro-N-((3S,4S)-3-methylpiperidin-4-yl)-2-(p-tolyloxy)acetamide

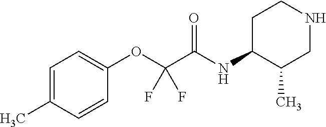 [SMILES: Cc1ccc(OC(F)(F)C(=O)N[C@H]2CCNC[C@@H]2C)cc1]

### Step A: tert-butyl (3S,4S)-4-(2,2-difluoro-2-(p-tolyloxy)acetamido)-3-methylpiperidine-1-carboxylate

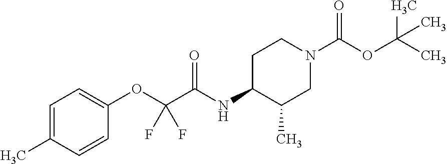 [SMILES: Cc1ccc(OC(F)(F)C(=O)N[C@H]2CCN(C(=O)OC(C)(C)C)C[C@@H]2C)cc1]

A solution of 2,2-difluoro-2-(p-tolyloxy)acetic acid (150 mg, 0.742 mmol), tert-butyl (3S,4S)-4-amino-3-methylpiperidine-1-carboxylate (191 mg, 0.890 mmol), HATU (339 mg, 0.890 mmol) and Et₃N (414 μL, 2.97 mmol) in DMA (2.99 mL) was stirred at room temperature overnight. The reaction mixture was diluted with MeOH, filtered through a hydrophilic PTFE 0.45 m Millipore® filter and purified by preparative HPLC (Phenomenex Gemini® NX—C18, 5 μm, ID 30 mm×100 mm) using a gradient of 10-100% ACN in water (Acid Mode) to give the title compound as a white solid (153 mg, 52%). ESI-MS m/z [M+Na]⁺ 421.5.

### Step B: 2,2-difluoro-N-((3S,4S)-3-methylpiperidin-4-yl)-2-(p-tolyloxy)acetamide

A solution of tert-butyl (3S,4S)-4-(2,2-difluoro-2-(p-tolyloxy)acetamido)-3-methylpiperidine-1-carboxylate (153 mg, 0.384 mmol) in DCM (960 μL) was treated with HCl in dioxane (4 M, 960 μL, 3.84 mmol) dropwise via syringe at room temperature. The reaction mixture was stirred at RT for 16 hours. The solvent was removed under reduced pressure and the product was lyophilized for 3 days to give an HCl salt of the title compound as a white solid (128 mg, quantitative). ¹H NMR (400 MHz, DMSO-d₆) δ ppm 0.78 (d, J=6.5 Hz, 3H), 1.66-1.86 (m, 2H), 1.92-2.02 (m, 1H), 2.27-2.35 (m, 3H), 2.64-2.76 (m, 1H), 2.91-3.03 (m, 1H), 3.19-3.28 (m, 2H), 3.60-3.72 (m, 1H), 7.11-7.17 (m, 2H), 7.21-7.27 (m, 2H), 8.88 (br s, 1H), 9.12 (br d, J=8.5 Hz, 1H); ESI-MS m/z [M+H]⁺ 299.3.

### Example 70: 2-(2-chlorophenoxy)-2,2-difluoro-N-(piperidin-4-yl)acetamide

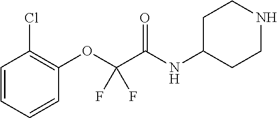 [SMILES: O=C(NC1CCNCC1)C(F)(F)Oc1ccccc1Cl]

To a solution of 2-(2-chlorophenoxy)-2,2-difluoroacetic acid (150 mg, 0.674 mmol), tert-butyl 4-aminopiperidine-1-carboxylate (135 mg, 0.674 mmol), and HATU (308 mg, 0.809 mmol) in DMA (1.35 mL) was added DIPEA (0.258 mL, 1.48 mmol). The reaction mixture was stirred at room temperature for 1 hour and then diluted with water (3 mL). The aqueous later was decanted off and the oily residue was washed with water (1 mL) and dried under a stream of nitrogen. The resulting oil was treated with HCl in dioxane (4 M, 1.69 mL, 6.74 mmol). The mixture was stirred at room temperature for 2 hours and then concentrated in vacuo and purified by preparative HPLC (Phenomenex Gemini® NX—C18, 5 μm, ID 30 mm×150 mm) using a gradient of 20-60% water/ACN in water (Basic Mode) to give the title compound as a white solid (59 mg, 29%). ¹H NMR (400 MHz, DMSO-d₆) δ ppm 1.34 (qd, J=11.9, 3.6 Hz, 2H), 1.55 (br d, J=10.2 Hz, 2H), 2.25-2.41 (m, 2H), 2.85 (br d, J=12.3 Hz, 2H), 3.51-3.66 (m, 1H), 7.25-7.31 (m, 1H), 7.31-7.38 (m, 2H), 7.54 (dt, J=7.8, 1.0 Hz, 1H), 8.88 (br d, J=7.6 Hz, 1H); ESI-MS m/z [M+H]⁺ 305.2.

### Example 71: 2-(2-chlorophenoxy)-2,2-difluoro-N-((3S,4S)-3-fluoropiperidin-4-yl)acetamide

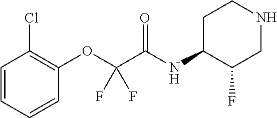 [SMILES: O=C(N[C@H]1CCNC[C@@H]1F)C(F)(F)Oc1ccccc1Cl]

To a solution of 2-(2-chlorophenoxy)-2,2-difluoroacetic acid (150 mg, 0.674 mmol), tert-butyl (3S,4S)-4-amino-3-fluoropiperidine-1-carboxylate (147 mg, 0.674 mmol) and HATU (308 mg, 0.809 mmol) in DMA (1.35 mL) was added DIPEA (0.258 mL, 1.48 mmol). The reaction mixture was stirred at room temperature for 1 hour and then diluted with water (3 mL). The aqueous later was decanted off and the oily residue was washed with water (1 mL) and dried under a stream of nitrogen. The resulting oil was treated with HCl in dioxane (4 M, 1.69 mL, 6.74 mmol). The mixture was stirred at room temperature for 2 hours and then concentrated in vacuo and purified by preparative HPLC (Phenomenex Gemini® NX—C18, 5 μm, ID 30 mm×150 mm) using a gradient of 20-60% water/ACN in water (Basic Mode) to give the title compound as a white solid (75 mg, 34%). ¹H NMR (400 MHz, DMSO-d₆) δ ppm 1.41 (qd, J=12.3, 4.4 Hz, 1H), 1.59-1.70 (m, 1H), 2.14-2.29 (m, 1H), 2.30-2.41 (m, 2H), 2.75 (br d, J=12.3 Hz, 1H), 3.08-3.18 (m, 1H), 3.71-3.84 (m, 1H), 4.23-4.50 (m, 1H), 7.26-7.32 (m, 1H), 7.36 (d, J=3.9 Hz, 2H), 7.55 (d, J=7.9 Hz, 1H), 9.18 (br d, J=7.9 Hz, 1H); ESI-MS m/z [M+H]⁺ 323.2.

Table 1 lists biological assay data (SSTR4 activity, SSTR4 binding, and SSTR1 binding) for some of the compounds shown in the examples, where larger pEC₅₀ and pIC₅₀ values represent higher activity or potency. The compounds shown in Table 1 were tested in accordance with a cell-based assay which measures the inhibition of forskolin stimulated cAMP in cells overexpressing SSTR4 (reported as pEC₅₀). Many of the compounds shown in Table 1 were also tested in accordance with membrane-based assays which measure competitive binding of the compounds to SSTR4 and SSTR1 (reported as pIC₅₀). These assays are described in the section entitled Biological Activity, above.

TABLE 1
Biological Assay Data

| | SSTR4 Activity | SSTR4 Binding | SSTR1 Binding |
|---|---|---|---|
| Example No. | pEC₅₀ | pIC₅₀ | pIC₅₀ |
| 1 | 7.34 | 6.66 | 4.69 |
| 2 | 7.46 | — | — |
| 3 | 7.90 | 7.04 | 5.27 |
| 4 | 7.90 | 6.46 | 4.45 |
| 5 | 8.05 | 7.41 | <4.3 |
| 6 | 7.50 | 6.55 | 5.08 |
| 7 | 6.75 | — | — |
| 8 | 6.74 | — | — |
| 9 | 6.74 | — | — |
| 10 | 7.15 | 6.29 | 4.81 |
| 11 | 6.85 | — | — |
| 12 | 7.24 | 6.05 | 4.8 |
| 13 | 8.56 | 7.45 | 5.06 |
| 14 | 7.17 | 6.39 | <4.3 |
| 15 | 9.08 | 7.83 | 5.2 |
| 16 | 8.86 | 7.44 | 4.88 |
| 17 | 7.05 | — | — |
| 18 | 8.70 | 7.58 | 5.33 |
| 19 | 7.12 | 6.51 | <4.3 |
| 20 | 7.11 | — | — |
| 21 | 7.37 | — | — |
| 22 | 7.83 | 7.39 | <4.3 |
| 23 | 7.63 | 7.13 | 4.37 |
| 24 | 7.71 | 7.23 | <4.3 |
| 25 | 8.58 | 7.33 | 4.47 |
| 26 | 7.83 | 7.15 | <4.3 |
| 27 | 7.99 | 7.25 | <4.3 |
| 28 | 6.71 | — | — |
| 29 | 7.03 | 6.73 | <4.3 |
| 30 | 7.75 | 7.39 | <4.3 |
| 31 | 8.18 | 7.75 | 4.67 |
| 32 | 7.30 | 5.49 | <4.3 |
| 33 | 7.78 | 6.79 | <4.3 |
| 34 | 7.32 | 6.17 | <4.3 |
| 35 | 7.58 | 6.66 | 5.23 |
| 36 | 7.63 | 6.56 | 5.33 |
| 37 | 7.08 | 6.49 | 5.44 |
| 38 | 7.85 | — | — |
| 39 | 6.99 | — | — |
| 40 | 6.93 | — | — |
| 41 | 7.25 | 6.53 | <4.3 |
| 42 | 6.99 | 6.09 | <4.3 |
| 43 | 7.20 | 6.87 | <4.3 |
| 44 | 7.69 | 7.06 | <4.3 |
| 45 | 7.43 | 7.68 | <4.3 |
| 46 | 7.93 | 7.29 | <4.3 |
| 47 | 7.91 | 7.59 | <4.3 |
| 48 | 7.04 | 5.43 | <4.3 |
| 49 | 7.67 | 6.46 | <4.3 |
| 50 | 7.24 | 5.88 | <4.3 |
| 51 | 7.45 | 5.53 | <4.3 |
| 52 | 6.77 | — | — |
| 53 | 7.26 | 5.43 | <4.3 |
| 54 | 7.67 | 6.55 | <4.3 |
| 55 | 8.28 | 7.13 | <4.3 |
| 56 | 8.77 | 7.14 | 4.35 |
| 57 | 8.95 | 7.41 | 4.53 |
| 58 | 8.30 | 6.77 | 4.33 |
| 59 | 7.23 | — | — |
| 60 | 6.76 | — | — |
| 61 | 6.91 | | |
| 62 | 6.78 | 6.25 | <4.3 |
| 63 | 6.73 | — | — |
| 64 | 8.88 | 7.25 | 4.72 |
| 65 | 8.60 | 7.82 | 4.66 |
| 66 | 9.97 | 6.95 | 4.89 |
| 67 | 6.88 | — | — |
| 68 | 7.47 | 6.94 | <4.3 |
| 69 | 8.21 | 6.99 | 4.34 |
| 70 | 7.74 | 6.48 | <4.3 |
| 71 | 7.80 | 6.78 | <4.3 |

As used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds. The above description is intended to be illustrative and not restrictive. Many embodiments will be apparent to those of skill in the art upon reading the above description. Therefore, the scope of the invention should be determined with reference to the appended claims and includes the full scope of equivalents to which such claims are entitled. The disclosures of all articles and references cited in the disclosure, including patents, patent applications and publications, are herein incorporated by reference in their entirety and for all purposes.

What is claimed is:

1. A compound of Formula 1, 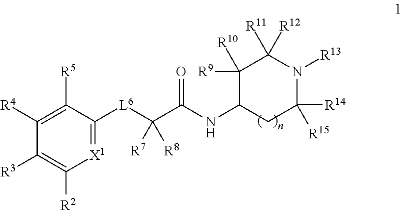
or a pharmaceutically acceptable salt thereof in which:
X¹ is selected from N and CR¹;
n is selected from 0 and 1;
R¹, R², R³, R⁴ and Rare each independently selected from
(i) hydrogen, halo, hydroxy and cyano; and
(ii) C₁₋₃ alkyl, C₁₋₃ alkoxy and C₃₋₆ cycloalkyl, each unsubstituted or substituted with 1 to 3 substituents independently selected from halo;
L⁶ is selected from —CH₂—, —N(R⁶)—, *—N(R⁶)CH₂— and —O—, wherein R⁶ is selected from hydrogen and C₁₋₃ alkyl, and * represents the point of attachment to an aromatic ring carbon atom;
R⁷ and R⁸ are each independently selected from halo and C₁₋₃ alkyl, provided R⁷ and R⁸ are not both methyl; or R⁷ and R⁸, together with the carbon atom to which they are attached, form a cyclopropylidene;
R⁹ and R¹⁰ are each independently selected from
(i) hydrogen and halo; and
(ii) C₁₋₃ alkyl and phenyl, each unsubstituted or substituted with 1 to 3 substituents independently selected from halo; or
R⁹ and R¹⁰, together with the carbon atom to which they are attached, form a cyclopropylidene;
R¹¹ and R¹² are each independently selected from hydrogen, halo and C₁₋₃ alkyl which is unsubstituted or substituted with 1 to 3 substituents independently selected from halo, provided no more than one of R¹¹ and R¹² is methyl; and
(a) R¹³ is selected from hydrogen and C₁₋₃ alkyl; and
R¹⁴ and R¹⁵ are each independently selected from hydrogen, halo, and C₁₋₃ alkyl which is unsubstituted or substituted with 1 to 3 substituents independently selected from halo, provided no more than one of R¹⁴ and R¹⁵ is methyl; or
(b) R¹³ and R¹⁴ together form a propane-1,3-diyl bridging the nitrogen and carbon atoms to which they are respectively attached; and
R¹⁵ is selected from hydrogen, halo, and C₁₋₃ alkyl which is unsubstituted or substituted with 1 to 3 substituents independently selected from halo;
provided
(i) if X¹ is CR¹, L⁶ is —CH₂—, and R⁷ and R⁸ form a cyclopropylidene, then at least one of R¹, R², R³, R⁴, R⁵, R⁹, R¹⁰, R¹¹, R¹², R¹³, R¹⁴ and R¹⁵ is not hydrogen;
(ii) if X¹ is CR¹, L⁶ is —CH₂—, n is 0, R¹, R², R⁴, R⁵, R¹¹, R¹², R¹⁴ and R¹⁵ are each hydrogen, R⁷ and R⁸ form a cyclopropylidene, and R⁹, R¹⁰ and R¹³ are each methyl, then R³ is not fluoro or hydroxy;
(iii) if X¹ is CR¹, L⁶ is —CH₂—, n is 0, R¹ is fluoro, R², R³, R⁴, R⁵, R¹¹, R¹², R¹⁴ and R¹⁵ are each hydrogen, and R⁷ and R⁸ form a cyclopropylidene, then R⁹, R¹⁰ and R¹³ are not all hydrogen and not all methyl;
(iv) if X¹ is CR¹, L⁶ is —CH₂—, n is 1, R¹, R², R³, R⁴, R⁵, R¹⁰, R¹¹, R¹², R¹³, R¹⁴ and R¹⁵ are each hydrogen, and R⁷ and R⁸ form a cyclopropylidene, then R⁹ is not methyl;
(v) if X¹ is CR¹, L⁶ is —CH₂—, n is 1, R¹, R², R³, R⁴, R⁵, R⁹, R¹⁰, R¹², R¹³, R¹⁴ and R¹⁵ are each hydrogen, and R⁷ and R⁸ form a cyclopropylidene, then R¹¹ is not methyl;
(vi) if X¹ is CR¹, n is 0, R¹, R², R³, R⁴, R⁵, R¹¹, R¹², R¹⁴ and R¹⁵ are each hydrogen, R⁷ and R⁸ form a cyclopropylidene, R⁹ and R¹⁰ are each methyl, and R¹³ is ethyl, then L⁶ is not —O— or —NH—;
(vii) if X¹ is CR¹, L⁶ is —CH₂—, n is 0, R¹, R², R⁴, R⁵, R¹¹, R¹², R¹⁴ and R¹⁵ are each hydrogen, R⁷ and R⁸ form a cyclopropylidene, R⁹ and R¹⁰ are both methyl, and R¹³ is ethyl, then R³ is not hydroxy;
(viii) if X¹ is CR¹, L⁶ is —CH₂—, n is 1, R⁷ and R⁸ form a cyclopropylidene, and R¹³ and R¹⁴ together form a propane-1,3-diyl bridging the nitrogen and carbon atoms to which they are respectively attached, then at least one of R¹, R², R³, R⁴, R⁵, R⁹, R¹⁰, R¹¹, R¹² and R¹⁵ is not hydrogen;
(ix) if X¹ is CR¹, L⁶ is —CH₂—, n is 1, R⁷ and R⁸ form a cyclopropylidene, and R¹³ and R¹⁴ together form a propane-1,3-diyl bridging the nitrogen and carbon atoms to which they are respectively attached, and R², R³, R⁴, R⁵, R⁹, R¹⁰, R¹¹, R¹² and R¹⁵ are each hydrogen, then R¹ is not fluoro; and
(x) if X¹ is CR¹, L⁶ is —CH₂—, n is 1, R⁷ and R⁸ form a cyclopropylidene, and R¹³ and R¹⁴ together form a propane-1,3-diyl bridging the nitrogen and carbon atoms to which they are respectively attached, and R¹, R², R⁴, R⁵, R⁹, R¹⁰, R¹¹, R¹² and R¹⁵ are each hydrogen, then R³ is not fluoro.

2. The compound or pharmaceutically acceptable salt according to claim 1, wherein X¹ is CR¹.

3. The compound or pharmaceutically acceptable salt according to any one of claims 1 to 2, wherein R¹ is selected from
(i) hydrogen, halo and cyano; and
(ii) C₁₋₃ alkyl, C₁₋₃ alkoxy and C₃₋₆ cycloalkyl, each unsubstituted or substituted with 1 to 3 substituents independently selected from halo.

4. The compound or pharmaceutically acceptable salt according to any one of claims 1 to 2, wherein R¹ is selected from hydrogen, halo, cyano, methyl, methoxy and cyclopropyl.

5. The compound or pharmaceutically acceptable salt according to claim 1, wherein X¹ is N.

6. The compound or pharmaceutically acceptable salt according to any one of claims 1 to 5, wherein R² is selected from
(i) hydrogen and halo; and
(ii) C₁₋₃ alkyl and C₁₋₃ alkoxy, each unsubstituted or substituted with 1 to 3 substituents independently selected from halo.

7. The compound or pharmaceutically acceptable salt according to any one of claims 1 to 5, wherein R² is selected from hydrogen, halo, methyl and methoxy.

8. The compound or pharmaceutically acceptable salt according to any one of claims 1 to 7, wherein R³ is selected from
(i) hydrogen and halo; and
(ii) C₁₋₃ alkyl which is unsubstituted or substituted with 1 to 3 substituents independently selected from halo.

9. The compound or pharmaceutically acceptable salt according to any one of claims 1 to 7, wherein R³ is selected from hydrogen, halo and methyl.

10. The compound or pharmaceutically acceptable salt according to any one of claims 1 to 9, wherein R⁴ is selected from hydrogen, halo, methyl and methoxy.

11. The compound or pharmaceutically acceptable salt according to any one of claims 1 to 9, wherein R⁴ is selected from hydrogen and fluoro.

12. The compound or pharmaceutically acceptable salt according to any one of claims 1 to 11, wherein R⁵ is selected from hydrogen, halo, cyano, methyl, methoxy and cyclopropyl.

13. The compound or pharmaceutically acceptable salt according to any one of claims 1 to 11, wherein R⁵ is hydrogen.

14. The compound or pharmaceutically acceptable salt according to any one of claims 1 to 13, wherein L⁶ is selected from —CH₂— and —O—.

15. The compound or pharmaceutically acceptable salt according to any one of claims 1 to 14, wherein R⁷ and R⁸ are each independently selected from fluoro and C₁₋₃ alkyl, provided R⁷ and R⁸ are not both methyl.

16. The compound or pharmaceutically acceptable salt according to any one of claims 1 to 14, wherein R⁷ and R⁸ are both fluoro.

17. The compound or pharmaceutically acceptable salt according to any one of claims 1 to 14, wherein R⁷ and R⁸, together with the carbon atom to which they are attached, form a cyclopropylidene.

18. The compound or pharmaceutically acceptable salt according to any one of claims 1 to 17, wherein R⁹ and R¹⁰ are each independently selected from hydrogen, halo, C₁₋₃ alkyl and phenyl.

19. The compound or pharmaceutically acceptable salt according to any one of claims 1 to 17, wherein R⁹ and R¹⁰, together with the carbon atom to which they are attached, form a cyclopropylidene.

20. The compound or pharmaceutically acceptable salt according to any one of claims 1 to 19, wherein R¹¹ and R¹² are each independently selected from hydrogen, halo and methyl.

21. The compound or pharmaceutically acceptable salt according to any one of claims 1 to 20, wherein R¹³ is selected from hydrogen and C₁₋₃ alkyl.

22. The compound or pharmaceutically acceptable salt according to any one of claims 1 to 20, wherein R¹³ is selected from hydrogen and methyl.

23. The compound or pharmaceutically acceptable salt according to any one of claims 21 and 22, wherein R¹⁴ and R¹⁵ are both hydrogen.

24. The compound or pharmaceutically acceptable salt according to any one of claims 1 to 20, wherein R¹³ and R¹⁴ together form a propane-1,3-diyl bridging the nitrogen and carbon atoms to which they are respectively attached.

25. The compound or pharmaceutically acceptable salt according to claim 24, wherein R¹⁵ is hydrogen.

26. The compound or pharmaceutically acceptable salt according to any one of claims 1 to 25, wherein n is 0.

27. The compound or pharmaceutically acceptable salt according to any one of claims 1 to 25, wherein n is 1.

28. The compound according to claim 1, which is selected from the following compounds:
(R)-1-(3-methylbenzyl)-N-(1-methylpyrrolidin-3-yl)cyclopropane-1-carboxamide;
N-(1-methyl-4-phenylpyrrolidin-3-yl)-1-(3-methylbenzyl)cyclopropane-1-carboxamide;
N-(trans-4-isopropyl-1-methylpyrrolidin-3-yl)-1-(3-methylbenzyl)cyclopropane-1-carboxamide;
N-(1,3-dimethylpiperidin-4-yl)-1-(3-methylbenzyl)cyclopropane-1-carboxamide;
N-((3S,4S)-3-fluoropiperidin-4-yl)-1-(3-methylbenzyl)cyclopropane-1-carboxamide;
N-(1,4-dimethylpyrrolidin-3-yl)-1-(3-methylbenzyl)cyclopropane-1-carboxamide;
1-(2-chlorophenoxy)-N-(trans-3-ethyl-1-methylpiperidin-4-yl)cyclopropane-1-carboxamide;
1-(2-chlorophenoxy)-N-(1,2-dimethylpiperidin-4-yl)cyclopropane-1-carboxamide;
1-(2-chlorophenoxy)-N-(hexahydro-1H-pyrrolizin-1-yl)cyclopropane-1-carboxamide;
N-(trans-1,3-dimethylpiperidin-4-yl)-1-(4-fluorobenzyl)cyclopropane-1-carboxamide;
N-(trans-1,3-dimethylpiperidin-4-yl)-1-(4-methylbenzyl)cyclopropane-1-carboxamide;
N-(trans-1,3-dimethylpiperidin-4-yl)-1-(2-methylbenzyl)cyclopropane-1-carboxamide;
1-(4-fluorobenzyl)-N-((3S,4S)-3-methylpiperidin-4-yl)cyclopropane-1-carboxamide;
2,2-difluoro-2-phenoxy-N-(piperidin-4-yl)acetamide;
1-(3-chlorobenzyl)-N-((3S,4S)-3-methylpiperidin-4-yl)cyclopropane-1-carboxamide;
1-(2-methylbenzyl)-N-((3S,4S)-3-methylpiperidin-4-yl)cyclopropane-1-carboxamide;
1-(2-chlorobenzyl)-N-(trans-1,3-dimethylpiperidin-4-yl)cyclopropane-1-carboxamide;
1-(4-chlorobenzyl)-N-((3S,4S)-3-methylpiperidin-4-yl)cyclopropane-1-carboxamide;
N-(trans-1,3-dimethylpiperidin-4-yl)-1-(3-fluorobenzyl)cyclopropane-1-carboxamide;
N-(trans-3-ethyl-1-methylpiperidin-4-yl)-1-(3-methylbenzyl)cyclopropane-1-carboxamide;
1-(4-fluorobenzyl)-N-((3S,4S)-3-fluoropiperidin-4-yl)cyclopropane-1-carboxamide;
1-(4-chlorobenzyl)-N-((3S,4S)-3-fluoropiperidin-4-yl)cyclopropane-1-carboxamide;
N-((3S,4S)-3-fluoropiperidin-4-yl)-1-(2-methylbenzyl)cyclopropane-1-carboxamide;
2,2-difluoro-N-((3S,4S)-3-methylpiperidin-4-yl)-2-phenoxyacetamide;
2,2-difluoro-N-((3S,4S)-3-methylpiperidin-4-yl)-3-phenylpropanamide;
1-(3-chlorobenzyl)-N-((3S,4S)-3-fluoropiperidin-4-yl)cyclopropane-1-carboxamide;
2,2-difluoro-N-(1-methylpiperidin-4-yl)-2-phenoxyacetamide;
2,2-difluoro-N-((3S,4S)-3-fluoropiperidin-4-yl)-2-phenoxyacetamide;
1-(4-fluoro-2-methylbenzyl)-N-((3S,4S)-3-fluoropiperidin-4-yl)cyclopropane-1-carboxamide;
1-(4-fluoro-3-methylbenzyl)-N-((3S,4S)-3-fluoropiperidin-4-yl)cyclopropane-1-carboxamide;
2-(2-cyanophenoxy)-2,2-difluoro-N-(piperidin-4-yl)acetamide;
2-(2-cyanophenoxy)-2,2-difluoro-N-((3S,4S)-3-methylpiperidin-4-yl)acetamide;
2,2-difluoro-N-(piperidin-4-yl)-2-(p-tolyloxy)acetamide;
N-((3S,4S)-1,3-dimethylpiperidin-4-yl)-1-(4-fluorobenzyl)cyclopropane-1-carboxamide;
1-(3-chlorobenzyl)-N-((3S,4S)-1,3-dimethylpiperidin-4-yl)cyclopropane-1-carboxamide;
1-(4-chlorobenzyl)-N-((3S,4S)-1,3-dimethylpiperidin-4-yl)cyclopropane-1-carboxamide;
N-((3S,4S)-1,3-dimethylpiperidin-4-yl)-1-(2-methylbenzyl)cyclopropane-1-carboxamide;
1-(4-chlorobenzyl)-N-((3S,4S)-3-fluoro-1-methylpiperidin-4-yl)cyclopropane-1-carboxamide;
N-((3S,4S)-1,3-dimethylpiperidin-4-yl)-2,2-difluoro-2-phenoxyacetamide;
1-(((6-chloropyridin-2-yl)(methyl)amino)methyl)-N-((2R,4R)-1,2-dimethylpiperidin-4-yl)cyclopropane-1-carboxamide;
(R)-2,2-difluoro-2-phenoxy-N-(5-azaspiro[2.4]heptan-7-yl)acetamide;
1-(2-fluorobenzyl)-N-((3S,4S)-3-fluoropiperidin-4-yl)cyclopropane-1-carboxamide;
1-(3-fluorobenzyl)-N-((3S,4S)-3-fluoropiperidin-4-yl)cyclopropane-1-carboxamide;
1-benzyl-N-((3S,4S)-3-fluoropiperidin-4-yl)cyclopropane-1-carboxamide;
N-((3S,4S)-3-fluoropiperidin-4-yl)-1-(3-methoxybenzyl)cyclopropane-1-carboxamide;
N-((3S,4S)-3-fluoropiperidin-4-yl)-1-(2-methoxybenzyl)cyclopropane-1-carboxamide;
2,2-difluoro-N-((2S,4S)-2-methylpiperidin-4-yl)-2-phenoxyacetamide;
2,2-difluoro-2-phenoxy-N-(5-azaspiro[2.5]octan-8-yl)acetamide;
2,2-difluoro-N-(cis-2-methylpiperidin-4-yl)-2-phenoxyacetamide;
2,2-difluoro-N-((2R,4R)-2-methylpiperidin-4-yl)-2-phenoxyacetamide;
2,2-difluoro-N-((2R,4S)-2-methylpiperidin-4-yl)-2-phenoxyacetamide;
2,2-difluoro-N-((2S,4R)-2-methylpiperidin-4-yl)-2-phenoxyacetamide;
2,2-difluoro-2-(2-fluorophenoxy)-N-((3S,4S)-3-methylpiperidin-4-yl)acetamide;
2,2-difluoro-2-(3-fluorophenoxy)-N-((3S,4S)-3-methylpiperidin-4-yl)acetamide;
2,2-difluoro-N-((3S,4S)-3-methylpiperidin-4-yl)-2-(o-tolyloxy)acetamide;
2,2-difluoro-N-((3S,4S)-3-methylpiperidin-4-yl)-2-(m-tolyloxy)acetamide;
2,2-difluoro-2-(4-fluorophenoxy)-N-((3S,4S)-3-methylpiperidin-4-yl)acetamide;
1-(3-chlorophenoxy)-N-((3S,4S)-3-fluoropiperidin-4-yl)cyclopropane-1-carboxamide;
1-(2-cyclopropylphenoxy)-N-((3S,4S)-3-fluoropiperidin-4-yl)cyclopropane-1-carboxamide;
2-(2-chlorophenoxy)-2,2-difluoro-N-((3S,4S)-3-methylpiperidin-4-yl)acetamide;
2-(3-chlorophenoxy)-2,2-difluoro-N-((3S,4S)-3-methylpiperidin-4-yl)acetamide;
2-(4-chlorophenoxy)-2,2-difluoro-N-((3S,4S)-3-methylpiperidin-4-yl)acetamide;
1-(3-fluoro-5-methoxybenzyl)-N-((3S,4S)-3-fluoropiperidin-4-yl)cyclopropane-1-carboxamide;
2,2-difluoro-N-((3S,4S)-3-methylpiperidin-4-yl)-2-(p-tolyloxy)acetamide;
2-(2-chlorophenoxy)-2,2-difluoro-N-(piperidin-4-yl)acetamide;
2-(2-chlorophenoxy)-2,2-difluoro-N-((3S,4S)-3-fluoropiperidin-4-yl)acetamide; and
a pharmaceutically acceptable salt of any one of the aforementioned compounds.

29. A compound which is selected from the following compounds:
3-(4-chlorophenyl)-2,2-dimethyl-N-((3S,4S)-3-methylpiperidin-4-yl)propanamide;
2-(2,3-dichlorophenoxy)-N-((3S,4S)-3-fluoropiperidin-4-yl)propanamide;
2-(2-chlorophenoxy)-N-((3S,4S)-3-fluoropiperidin-4-yl)propanamide;
2-(2-cyclopropylphenoxy)-N-((3S,4S)-3-fluoropiperidin-4-yl)propanamide;
2-(2-chlorophenoxy)-N-((2R,4R)-1,2-dimethylpiperidin-4-yl)propanamide; and
a pharmaceutically acceptable salt of any one of the aforementioned compounds.

30. A compound or pharmaceutically acceptable salt as defined in any one of claims 1 to 29 for use as a medicament.

31. A compound of Formula 1, 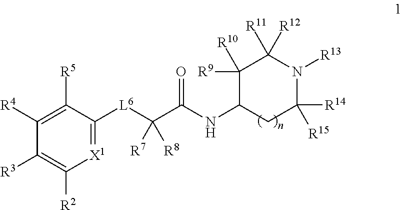
or a pharmaceutically acceptable salt thereof for use as a medicament in which:
X¹ is selected from N and CR¹;
n is selected from 0 and 1;
R¹, R², R³, R⁴ and R⁵ are each independently selected from
(i) hydrogen, halo, hydroxy and cyano; and
(ii) C₁₋₃ alkyl, C₁₋₃ alkoxy and C₃₋₆ cycloalkyl, each unsubstituted or substituted with 1 to 3 substituents independently selected from halo;
L⁶ is selected from —CH₂—, —N(R⁶)—, *—N(R⁶)CH₂— and —O—, wherein R⁶ is selected from hydrogen and C₁₋₃ alkyl, and * represents the point of attachment to an aromatic ring carbon atom;
R⁷ and R⁸ are each independently selected from hydrogen, halo and C₁₋₃ alkyl, wherein at least one of R⁷ and R⁸ is not hydrogen; or R⁷ and R⁸, together with the carbon atom to which they are attached, form a C₃₋₆ cycloalkylidene;
R⁹ and R¹⁰ are each independently selected from
(i) hydrogen and halo; and
(ii) C₁₋₃ alkyl and phenyl, each unsubstituted or substituted with 1 to 3 substituents independently selected from halo; or
R⁹ and R¹⁰, together with the carbon atom to which they are attached, form a cyclopropylidene;
R¹¹ and R¹² are each independently selected from hydrogen, halo and C₁₋₃ alkyl which is unsubstituted or substituted with 1 to 3 substituents independently selected from halo, provided no more than one of R¹¹ and R¹² is methyl; and
(a) R¹³ is selected from hydrogen and C₁₋₃ alkyl; and
R¹⁴ and R¹⁵ are each independently selected from hydrogen, halo, and C₁₋₃ alkyl which is unsubstituted or substituted with 1 to 3 substituents independently selected from halo, provided no more than one of R¹⁴ and R¹⁵ is methyl; or
(b) R¹³ and R¹⁴ together form a propane-1,3-diyl bridging the nitrogen and carbon atoms to which they are respectively attached; and
R¹⁵ is selected from hydrogen, halo, and C₁₋₃ alkyl which is unsubstituted or substituted with 1 to 3 substituents independently selected from halo;
provided
(i) if X is CR¹, n is 1, L⁶ is —O—, R⁷ is methyl and R⁸ is hydrogen, then at least one of R¹, R², R³, R⁴, R⁵, R⁹, R¹⁰, R¹¹, R¹², R¹³, R¹⁴ or R¹⁵ is not hydrogen;
(ii) if X is CR¹, n is 1, L⁶ is —O—, R⁷ and R⁸ are both methyl, and R¹, R², R⁴, R⁵, R⁹, R¹⁰, R¹¹, R¹², R¹³, R¹⁴ and R¹⁵ are each hydrogen, then R³ is not chloro; and
(iii) if X is CR¹, n is 1, L⁶ is —O—, R⁷ is methyl or ethyl, R¹, R², R³, R⁴, R⁵, R⁸, R¹⁰, R¹¹, R¹², R¹³, R¹⁴ and R¹⁵ are each hydrogen, then R⁹ is not unsubstituted phenyl.

32. The compound according to claim 31 or pharmaceutically acceptable salt thereof for use as a medicament, wherein R⁷ and R⁸ are each independently selected from halo and C₁₋₃ alkyl, provided R⁷ and R⁸ are not both methyl.

33. A pharmaceutical composition comprising:
a compound or pharmaceutically acceptable salt as defined in any one of claims 1 to 32; and
a pharmaceutically acceptable excipient.

34. A compound or pharmaceutically acceptable salt as defined in any one of claims 1 to 32 for use in treating a disease, disorder or condition selected from Alzheimer's disease, depression, anxiety, schizophrenia, bipolar disorder, autism, epilepsy, pain, and hyperactivity disorder.

35. A method of treating a disease, disorder or condition in a subject, the method comprising administering to the subject a compound or pharmaceutically acceptable salt as defined in any one of claims 1 to 32, wherein the disease, disorder or condition is selected from Alzheimer's disease, depression, anxiety, schizophrenia, bipolar disorder, autism, epilepsy, pain, and hyperactivity disorder.

36. A combination comprising a compound or pharmaceutically acceptable salt as defined in any one of claims 1 to 32, and at least one additional pharmacologically active agent.

37. The combination according to claim 36, wherein the additional pharmacologically active agent is selected from beta-secretase inhibitors, gamma-secretase inhibitors, HMG-CoA reductase inhibitors, nonsteroidal anti-inflammatory drugs, vitamin E, anti-amyloid antibodies, antidepressants, antipsychotics, anxiolytics, and anticonvulsants.
